(19) United States
(12) Patent Application Publication
(10) Pub. No.: US 2023/0277657 A1
(43) Pub. Date: Sep. 7, 2023

(54) MICROFLUIDIC MIXING DEVICE AND METHODS OF USE

(71) Applicant: GLAXOSMITHKLINE BIOLOGICALS SA, Rixensart (BE)

(72) Inventors: Philippe Raymond JEHOULET, Rixensart (BE); Amedeo MENAFRO, Rixensart (BE); Laurent STRODIOT, Rixensart (BE); Emanuele TOMBA, Siena (IT); Ioannis TSOUMPAS, Rixensart (BE)

(73) Assignee: GLAXOSMITHKLINE BIOLOGICALS SA, Rixensart (BE)

(21) Appl. No.: 17/923,252

(22) PCT Filed: May 4, 2021
(86) PCT No.: PCT/EP2021/061621

§ 371 Date: Nov. 4, 2022

(30) Foreign Application Priority Data
May 5, 2020 (EP) 20173050.4

(51) Int. Cl. A61K 39/39; A61K 9/127; A61K 9/08; B01F 33/30; B01F 25/431; B01F 23/45

(52) U.S. Cl. CPC A61K 39/39; A61K 9/1277; A61K 9/08; B01F 33/304; B01F 25/4317; B01F 23/45; A61K 2039/55555

(57) ABSTRACT

A microfluidic mixing device (101) comprising: a mixing chamber; one inlet channel (104) into the mixing chamber (102) for a first fluid and two inlet channels (103a, 103b) into the mixing chamber for a second fluid, said inlet channels being disposed substantially symmetrically at a proximal end (108) of the mixing chamber; at least one outlet (105) for mixed material at a distal end of the mixing chamber, wherein the mixing chamber comprises one or more baffles.

### TECHNICAL FIELD

The present invention relates to microfluidic devices of use in the manufacture of liposomal adjuvants, methods for manufacturing an adjuvant using such microfluidic devices and to related aspects.

### BACKGROUND OF THE INVENTION

Adjuvants are included in vaccines to improve humoral and cellular immune responses, particularly in the case of poorly immunogenic subunit vaccines. Similar to natural infections by pathogens, adjuvants rely on the activation of the innate immune system to promote long-lasting adaptive immunity. As simultaneous activation of multiple innate immune pathways is a feature of natural infections, adjuvants may combine multiple immunostimulants in order to promote adaptive immune responses to vaccination.

The Adjuvant System 01 (AS01) is a liposome-based adjuvant which contains two immunostimulants, 3-O-desacyl-4′-monophosphoryl lipid A (3D-MPL) and QS-21 (Garcon and Van Mechelen, 2011; Didierlaurent et al, 2017). The TLR4 agonist 3D-MPL is a non-toxic derivative of the lipopolysaccharide from Salmonella minnesota. QS-21 is a natural saponin fraction extracted from the bark of the South American tree Quillaja saponaria Molina (Kensil et al., 1991; Ragupathi et al., 2011). AS01 is included in the recently developed malaria vaccine RTS,S (Mosquirix™) and Herpes zoster HZ/su vaccine (Shingrix™) and in multiple candidate vaccines in development against pathogens such as human immunodeficiency virus and Mycobacterium tuberculosis. During preclinical and clinical evaluation of these candidate vaccines, both antigen-specific antibody and CD4⁺ T cell immunity were consistently observed. The ability of AS01 to consistently generate cellular immune responses to vaccination sets it apart from other adjuvants that typically mainly promote humoral responses to vaccination (Black et al., 2015; Garcon and Van Mechelen, 2011). Concomitantly, AS01-adjuvanted vaccines have been efficient in promoting immunogenicity to vaccination in challenging populations, such as infants (with RTS,S) and older adults (with HZ/su).

3D-MPL and QS-21 have been shown to act synergistically in the induction of immune responses. Furthermore, the manner in which both immunostimulants are provided has been shown to be an important factor which influences the quality of the induced responses, with the liposomal presentation in AS01 providing higher potency than the oil-in-water emulsion based AS02. (Dendouga et al. 2012).

Microfluidic devices may be used for mixing small volumes of fluids thereby conserving precious materials. However, they have generally been used in research environments to prepare small amounts of product. In order to find utility in an industrial production setting, there is a need for low cost microfluidic devices that simplify and ease manufacturing processes. In addition, for pharmaceutical purposes, such devices need to be able to reliably generate nanoparticles at high throughput whilst maintaining controlled size and polydispersity.

International patent application WO2018219521 relates to devices and methods for the manufacture of saponin containing liposomal adjuvants.

International patent application WO2020109365 (application number PCT/EP2019/082689) relates to the manufacture of TLR4 agonist containing adjuvants using microfluidics.

International patent application WO2020115178 (application number PCT/EP2019/083758) relates to microfluidic devices, such devices being of use for example in the manufacture of adjuvants.

Wong et al. 2003 describes the investigation of mixing in a cross-shaped micromixer with static mixing elements (SME) through the use of a computational fluid dynamics.

International patent application WO2015148764 describes microfluidic devices intended for use in quantification of analytes.

European patent application EP1810746 describes microfluidic devices intended to facilitate droplet formation on merging of incompatible fluids through a cyclically changing cross-sectional area.

There remains a need for new manufacturing approaches which enable the safe, convenient and cost-effective production of materials such as liposomal adjuvants on a commercially viable scale while maintaining the immunological performance arising from prior art manufacturing approaches.

### SUMMARY OF THE INVENTION

The present invention provides a microfluidic mixing device comprising: a mixing chamber; one inlet channel into the mixing chamber for a first fluid and two inlet channels into the mixing chamber for a second fluid, said inlet channels being disposed substantially symmetrically at a proximal end of the mixing chamber; at least one outlet for mixed material at a distal end of the mixing chamber; characterised in that the mixing chamber comprises one or more baffles.

Additionally, there is provided a method of manufacturing a liposomal adjuvant using a microfluidic device, comprising the following steps:
(a) mixing in the device a first solution comprising a solvent and a lipid, and a second solution comprising water; and
(b) removing the solvent.

The present invention also provides a method of manufacturing a liposomal concentrate of use in preparing a liposomal adjuvant using a microfluidic device, comprising the step of mixing in the device a first solution comprising a solvent and lipid, and a second solution comprising water.

Further provided are a liposomal adjuvant and a liposomal concentrate obtainable from, such as obtained from, the methods described herein.

### BRIEF DESCRIPTION OF THE DRAWINGS

FIG. 1: General microfluidic device configuration

FIG. 2: Schematic of a prior art microfluidic device from WO2018219521 (referred to as ‘Design 1’ herein). Dimensions are in mm.

FIG. 3: Computational fluid dynamics (CFD) simulation of the impact of the central channel configuration on fluid flow.

FIG. 4: Results of the computational fluid dynamics simulations for each design in Example 2 using the same total flow rate and flow rate ratio (total 16 ml/min, 4:1 External/Internal channels).

FIG. 5: Comparison of the mixing performance for each design in Example 2 (note that the lines for Designs 1 and 2 overlay precisely). The x axis corresponds to the position along length of the mixing chamber as a proportion of total chamber length and the y axis is the mixing co-efficient (Alpha/Alfa), with higher values of the mixing co-efficient indicating better mixing.

FIG. 6: Final mixing coefficient (Alpha/Alfa) as determined for each of the 19 designs in Example 3. The width of the mixing chamber (MC) was either 1 mm, 2 mm or 3 mm; the width of the internal channel (Capint) was either 0.1 mm, 0.2 mm or 0.3 mm; the width of the external channels (CapExt) was either 0.1 mm, 0.2 mm or 0.3 mm.

FIG. 7: Comparison of the mixing profile of the modified geometries from Example 4 versus Design 1 (from WO2018219521).

FIG. 8: Results from Example 5 relating to the impact of channel depth using four different depths: 0.4 mm, 0.5 mm, 0.6 mm and 0.675 mm. The x axis corresponds to the position along length of the mixing chamber as a proportion of total chamber length and the y axis is the mixing co-efficient (Alpha/Alfa), with higher values of the mixing co-efficient indicating better mixing.

FIG. 9: Overview of the liposomal adjuvant size (A) and PDI (B) obtained for each design from Example 6.

FIG. 10: General microfluidic device configuration incorporating baffles (A) and with reducing mixing chamber width incorporating baffles (B).

FIG. 11: Design details (Cases 1 to 10) and computational fluid dynamics simulation of the impact of various baffle arrangements from Example 7. (A) Table of parameters (B) CFD simulation.

FIG. 12: Comparison of the mixing profile for Cases 1 to 10 from Example 7.

FIG. 13: Design details (Cases 11 to 15) and computational fluid dynamics simulation of the impact of various baffle arrangements from Example 7. (A) Table of parameters (B) CFD simulation.

FIG. 14: Comparison of the mixing profile for Cases 11 to 15 from Example 7.

FIG. 15: Design details (Cases 16 to 21) and computational fluid dynamics simulation of the impact of various baffle arrangements from Example 7. (A) Table of parameters (B) CFD simulation.

FIG. 16: Comparison of the mixing profile for Cases 16 to 21 from Example 7.

FIG. 17: Design details (Cases 19b, 19c, 21b and 21c) and computational fluid dynamics simulation of the impact of various baffle arrangements from Example 7. (A) Table of parameters (B) CFD simulation.

FIG. 18: Comparison of the mixing profile for Cases 19 to 21 and 19b, 19c, 21b and 21c from Example 7.

FIG. 19: General microfluidic device configuration for Design 4-1 (A), computational fluid dynamics simulation for Design 4-1 (B) and comparison of the mixing profiles for Design 4 and Design 4-1 (C) from Example 7.

FIG. 20: General microfluidic device configuration incorporating different baffle shapes (A) and computational fluid dynamics simulation of the impact of baffle shapes (B) from Example 8.

FIG. 21: Comparison of the mixing profile for different baffle shapes from Example 8.

FIG. 22: Schematic for Design 4-1. Dimensions are in mm.

FIG. 23: Schematic for Design 6-4-3-1. Dimensions are in mm.

FIG. 24: Schematic for Design 6-4. Dimensions are in mm.

FIG. 25: Schematic for Design 6-5. Dimensions are in mm.

FIG. 26: Overview of the liposomal adjuvant size (A) and PDI (B) obtained for Designs 4-1, 6-4-3-1, 6-4 and 6-5 from Example 9 versus Design 1 (from WO2018219521).

FIG. 27: Liposomal adjuvant size and PDI obtained for Designs 4-1 and 6-5 at different total flow rates and temperatures from Example 9.

FIG. 28: Liposomal adjuvant size and PDI obtained for Design 6-5 at different total flow rates and temperatures from Example 9.

FIG. 29: Total flow rates, temperature and size design space obtained for Design 6-5 at different total flow rates and temperatures from Example 9

FIG. 30: Schematic drawing showing six different implementations (A to F) of the inlet region of the microfluidic device. In implementations C and E, the first ends of each of the two outer channels are continuous with one another and share a common inlet.

FIG. 31: Operational arrangement of two eight mixing chamber chips with external distribution and collection manifolds.

FIG. 32: Schematic of a commercial scale multichamber process for the manufacture of liposomal adjuvant

FIG. 33: Image of a multilayer 16 mixing chamber chip (based on design 6-5) with integrated inlet and outlet distribution manifolds and single connections for first fluid, second fluid and mixed material, as used in Example 10.

FIG. 34: Schematic of Design 16A—a multilayer 16 mixing chamber chip (layer 1) with integrated inlet distribution (layers 2 and 3) and outlet collection (layer 1) manifolds, as used in Example 10. The outlet manifold connects to mixing chamber ends through channels located in layer 2.

FIG. 35: Schematic of Design 16C—a multilayer 16 mixing chamber chip (layer 1) with integrated inlet distribution (layers 2 and 3) and outlet collection (layer 1) manifolds, as used in Example 10. The outlet manifold connects directly to mixing chamber ends.

### DETAILED DESCRIPTION

The present invention provides a microfluidic mixing device comprising: a mixing chamber; one inlet channel into the mixing chamber for a first fluid and two inlet channels into the mixing chamber for a second fluid, said inlet channels being disposed substantially symmetrically at a proximal end of the mixing chamber; at least one outlet for mixed material at a distal end of the mixing chamber; characterised in that the mixing chamber comprises one or more baffles.

### Microfluidic Devices

The term “microfluidic device” refers to a device with at least one channel having micron-scale dimensions (i.e., a dimension less than 1 mm) for manipulating (e.g., flowing, mixing, etc.) a fluid sample. Microfluidic devices of the present invention are passive devices, containing no moving parts and having no requirement for energy input other than the pressure used to drive fluid flow through the device.

The term “chip” refers to the structure in which the microfluidic device is located, typically a device is etched or moulded into a material such as glass, silicon or polymer such as polydimethylsiloxane. A chip may contain a plurality of microfluidic devices and may also integrate distribution and/or collection manifolds to assist in fluid distribution to and collection from the plurality of microfluidic devices contained within a chip. For convenience in manufacture, chips may be formed from a plurality of layers.

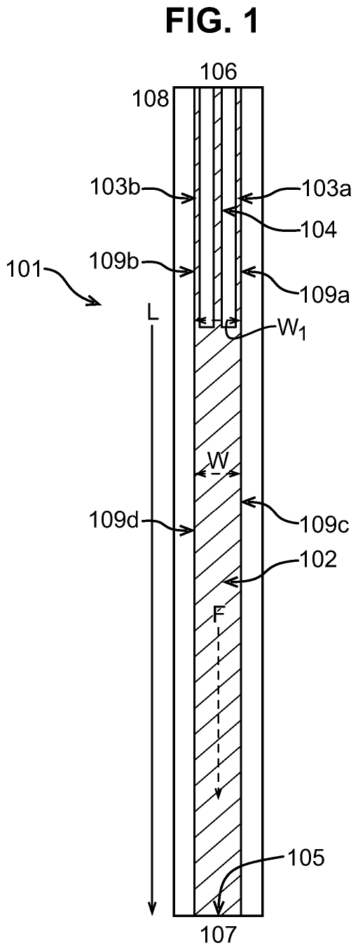

FIG. 1 provides a plan view of some general features of microfluidic devices (101) of the present invention. The device extends between a proximal end (108) comprising an inlet region (106) and a distal end (107) comprising an outlet region (105), wherein the inlet region comprises an inner inlet channel (104) for transport of a first fluid and two outer inlet channels (103a, 103b) for transport of a second fluid, said outer inlet channels (103a,103b) defined in part by a first outer wall (109a) and a second outer wall (109b) respectively, wherein the inner inlet channel is disposed between the two outer channels, wherein the inner inlet channel (104) and outer inlet channels (103a,103b) extend from the proximal end (108) to a mixing chamber (102) which extends from the distal end of the inlet region (106) to the proximal end of the outlet region (105), wherein the mixing chamber (102) is in flow communication with the inner and outer inlet channels (103a,103b,104) to receive the first and second fluids from the inner and outer inlet channels (103a,103b,104) and wherein the mixing chamber (102) has a width (W), the width (W) may optionally vary along the length (L) of the mixing chamber (102) or, for example, the width (W) may equal the width (W1) between the outer walls (109a, 109b) of the two outer channels (103a,103b). Particularly, the mixing chamber (102) is defined in part by a first outer wall (109c) and a second outer wall (109d) which are continuous with the respective outer walls (109a, 109b) of the two outer inlet channels (103a, 103b). More particularly, the outer inlet channel outer walls (109a, 109b) and mixing chamber outer walls (109c, 109d) may be provided by a first (109a,109c) and second wall (109b, 109d) which extend over substantially the whole length of the device (101) between the proximal end (106) and distal end (107). The general direction of flow is indicated shown (F).

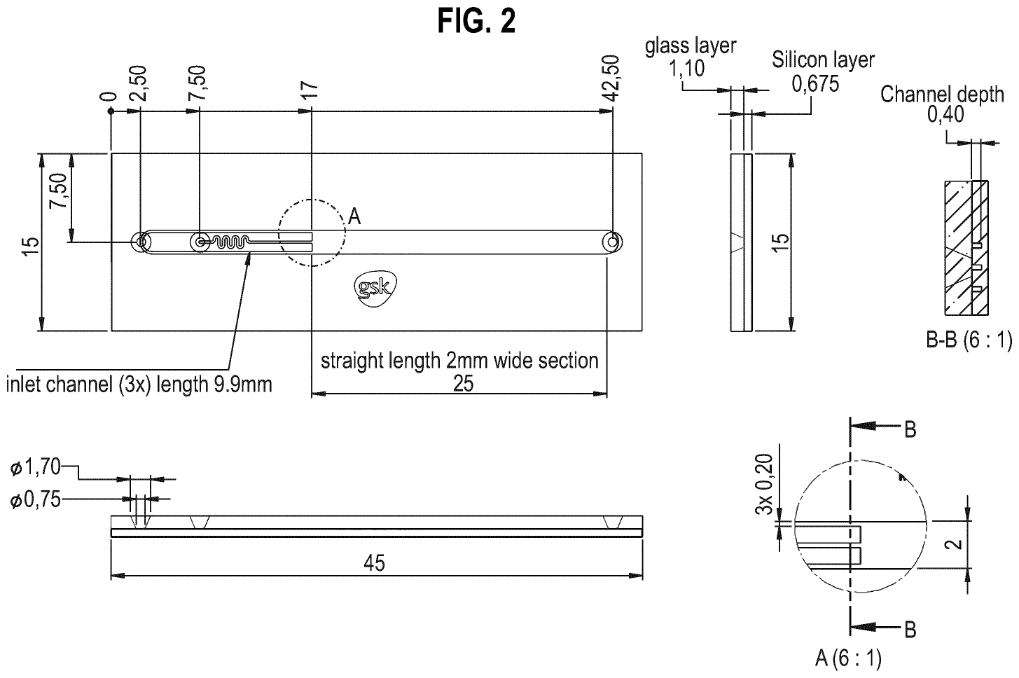

WO2018219521 describes, inter alia, the use of a microfluidic device as shown in FIG. 2 for the manufacture of liposomal adjuvants in high quantities while maintaining adequate size and polydispersity control. The present inventors have surprisingly found that mixing within such microfluidic devices can be further improved by the introduction of baffles to the mixing chamber. Improved mixing may in turn provide improved polydispersity.

The cross-section of the mixing chamber may be of any shape, though is substantially symmetrical, such as symmetrical. The cross-section may be substantially rectangular (such as square). The cross-section may be elongate in nature, with the larger dimension being at least twice that of the perpendicular dimension, such as at least three times or at least four times. The larger dimension may be no more than ten times that of the perpendicular dimension, such as no more than eight times or no more than six times. The larger dimension will usually be one to ten times that of the perpendicular dimension, such as two to eight times, especially three to five times, in particular three to four times. The larger dimension may be one and a half to four times that of the perpendicular dimension.

The mixing chamber should be of adequate length to allow for mixing to be substantially complete by the time liquid reaches the outlet region. Consequently, the optimal length of the mixing chamber may depend on the precise configuration of the inlets and operating conditions. Excessively long mixing chambers are wasteful of space; therefore, it is beneficial for the mixing chamber to be adequately but not excessively long.

Suitably the length of the mixing chamber is at least 15 mm, such as at least 17.5 mm, especially at least 20 mm, in particular at least 22 mm. Suitably the length of the mixing chamber is 100 mm or less, such as 75 mm or less, especially 50 mm or less, in particular 40 mm or less. The length of the mixing chamber may be 15 to 100 mm, such as 17.5 to 75 mm, especially 20 mm to 50 mm, in particular about 25 mm, such as 25 mm.

Suitably the mixing chamber is substantially rectangular in cross-section, such as rectangular.

Suitably the maximum width of the mixing chamber is 0.8 to 2.2 mm, such as 1 to 2 mm, especially 1.2 to 2 mm. Desirably the maximum width of the mixing chamber is 1.4 to 1.8 mm, in particular about 1.6 mm, such as 1.6 mm.

The maximum width of the mixing chamber may be 0.8 to 1.2 mm, in particular about 1 mm, such as 1 mm.

Suitably the minimum width of the mixing chamber is 0.8 to 2.2 mm, such as 1 to 2 mm, especially 1.2 to 2 mm. Desirably the minimum width of the mixing chamber is 1.4 to 1.8 mm, in particular about 1.6 mm, such as 1.6 mm.

The minimum width of the mixing chamber may be 0.8 to 1.2 mm, in particular about 1 mm, such as 1 mm.

The minimum width of the mixing chamber may be 0.4 to 1.2 mm, such as 0.6 to 0.9 mm, especially about 0.75 mm, such as 0.75 mm.

Suitably the maximum width of the mixing chamber and minimum width of the mixing chamber are the substantially the same (i.e. the mixing chamber has sides which are substantially equidistant along the mixing chamber length), such as the same. Alternatively, the mixing chamber is of reducing width along its length, such as reduced width by up to 50%.

Reductions in width may be continuous or discontinuous along the length of the mixing chamber. Continuous reductions in width along the length of the mixing chamber may be linear or non-linear.

Suitably the mixing chamber is at least 0.05 mm deep, such as at least 0.1 mm deep, especially at least 0.2 mm deep and in particular at least 0.3 mm deep. Desirably the mixing chamber is at least 0.4 mm deep. Suitably the mixing chamber is 10 mm deep or less, such as 5 mm deep or less, especially 2 mm deep or less and in particular 1 mm deep or less. Desirably the mixing chamber is 0.8 mm deep or less. The mixing chamber may be 0.1 to 2 mm deep, such as 0.3 to 0.8 mm deep, especially 0.4 to 0.6 mm deep and in particular about 0.5 mm deep such as 0.5 mm deep.

Suitably the mixing chamber is of substantially consistent depth along its length (i.e. the mixing chamber has a top and bottom which are substantially equidistant along the mixing chamber length), such as consistent depth. Alternatively, the mixing chamber is of reducing depth along its length, such as reduced depth by up to 50%.

Reductions in depth may be continuous or discontinuous along the length of the mixing chamber. Continuous reductions in depth along the length of the mixing chamber may be linear or non-linear.

Suitably the mixing chamber width at any point is 1 to 5 times the mixing chamber depth.

Suitably the mixing chamber has a cross-sectional area of 0.1 to 2.2 mm², such as 0.2 to 1.8 mm², especially 0.4 to 1.6 mm², in particular 0.6 to 1.0 mm², such as about 0.8 mm², such as 0.8 mm². The mixing chamber cross-sectional area may be 0.4 to 1.0 mm².

The mixing chamber may have a cross-sectional area of 0.2 to 0.8 mm², such as 0.3 to 0.7 mm², especially 0.4 to 0.6 mm², such as about 0.5 mm², such as 0.5 mm². The mixing chamber cross-sectional area may be 0.25 to 0.6 mm².

The microfluidic device will have one inlet to the mixing chamber for delivery of a first fluid, such as a first solution. The cross-section of the inlet may be of any shape, though is substantially symmetrical, such as symmetrical. The cross-section may be rectangular (such as square). The inlet channel for the first fluid will typically be substantially rectangular in cross-section.

Suitably the inlet channel for the first fluid is 0.1 to 0.7 mm wide, such as 0.15 to 0.5 mm wide, especially 0.15 to 0.5 mm wide and in particular 0.2 to 0.35 mm wide. Desirably the inlet channel for the first fluid is 0.25 to 0.3 mm wide, such as 0.27 mm wide.

Suitably the inlet channel for the first fluid is at least 0.05 mm deep, such as at least 0.1 mm deep, especially at least 0.2 mm deep and in particular at least 0.3 mm deep. Desirably the inlet channel for the first fluid is at least 0.4 mm deep. Suitably the inlet channel for the first fluid is 10 mm deep or less, such as 5 mm deep or less, especially 2 mm deep or less and in particular 1 mm deep or less. Desirably the inlet channel for the first fluid is 0.8 mm deep or less. The inlet channel for the first fluid may be 0.1 to 2 mm deep, such as 0.3 to 0.8 mm deep, especially 0.4 to 0.6 mm deep and in particular about 0.5 mm deep such as 0.5 mm deep.

Suitably the inlet channel for the first fluid has a cross-sectional area of 0.01 to 4 mm², such as 0.05 to 1 mm² and especially 0.08 to 0.4 mm². Desirably the inlet channel for the first fluid has a cross-sectional area of 0.1 to 0.2 mm², in particular about 0.135 mm², such as 0.135 mm².

Suitably the depth of the inlet channel for the first fluid is substantially the same as the depth of the mixing chamber, such as the same depth.

Suitably the direction of flow from the inlet channel for the first fluid into the mixing chamber is substantially parallel (e.g. within 15 degrees, such as within 10 degrees, in particular within 5 degrees) to the general direction of flow in the mixing chamber.

Suitably the inlet channel for the first fluid is substantially linear, such as linear, for at least 3 mm, such as at least 5 mm, especially at least 7 mm to the point at which it meets the mixing chamber.

The microfluidic device will have two inlets to the mixing chamber for delivery of a second fluid, such as a second solution. The cross-section of the inlets may be of any shape, though is substantially symmetrical, such as symmetrical. The cross-section may be rectangular (such as square). The inlet channels for the second fluid will typically be substantially rectangular in cross-section.

Suitably the inlet channels for the second fluid are 0.025 to 0.3 mm wide, such as 0.05 to 0.25 mm wide. Desirably the inlet channels for the second fluid are 0.08 to 0.12 mm wide, such as 0.1 mm wide.

Suitably the inlet channels for the second fluid are at least 0.05 mm deep, such as at least 0.1 mm deep, especially at least 0.2 mm deep and in particular at least 0.3 mm deep. Desirably the inlet channels for the second fluid are at least 0.4 mm deep. Suitably the inlet channels for the second fluid are 10 mm deep or less, such as 5 mm deep or less, especially 2 mm deep or less and in particular 1 mm deep or less. Desirably the inlet channels for the second fluid are 0.8 mm deep or less. The inlet channels for the second fluid may be 0.1 to 2 mm deep, such as 0.3 to 0.8 mm deep, especially 0.4 to 0.6 mm deep and in particular about 0.5 mm deep such as 0.5 mm deep.

Suitably each of the inlet channels for the second fluid has a cross-sectional area of 0.005 to 3 mm², such as 0.01 to 0.5 mm². Desirably each of the inlet channels for the second fluid has a cross-sectional area of 0.02 to 0.1 mm², in particular about 0.05 mm², such as 0.05 mm².

Suitably the depth of the inlet channels for the second fluid is substantially the same as the depth of the mixing chamber, such as the same depth.

Suitably the direction of flow from the inlet channels for the second fluid into the mixing chamber is substantially parallel (e.g. within 15 degrees, such as within 10 degrees, in particular within 5 degrees) to the general direction of flow in the mixing chamber.

Suitably the inlet channels for the second fluid are substantially linear, such as linear, for at least 3 mm, such as at least 5 mm, especially at least 7 mm and in particular at least 10 mm to the point at which they meet the mixing chamber.

Suitably the inlet channels for the second fluid are substantially identical in shape. Suitably the inlet channels for the second fluid are substantially identical in size. Desirably the inlet channels for the second fluid are substantially identical, such as identical.

The total cross-sectional area of all inlets will suitably be less than 70% of the cross-sectional area of the mixing chamber, such as less than 60% and especially less than 50%. The total cross-sectional area of all inlets may be 15% or more of the cross-sectional area of the mixing chamber, such as 20% or more % and especially 25% or more. The total cross-sectional area of all inlets may suitably be 20 to 60% of the cross-sectional area of the mixing chamber, such as 25 to 50%.

By the term ‘said inlet channels being disposed substantially symmetrically at a proximal end of the mixing chamber’ means that all inlets are substantially symmetrically disposed at the proximal end of the mixing chamber.

FIG. 30 illustrates a number of possible inlet implementations.

The microfluidic device will have at least one outlet from the mixing chamber for recovery of the mixed material. The device may have a plurality of outlets from the mixing chamber for recovery of the mixed material, such as two or three outlets, which are later combined. Suitably the device will have a single outlet from the mixing chamber for recovery of the mixed material. The outlet(s) are located at the distal end of the mixing chamber.

The cross-section of the outlets may be of any shape, though is typically symmetrical. The cross-section may be rectangular (such as square), typically having an area of 0.1 to 1 mm², such as 0.2 to 0.8 mm², for example 0.4 to 0.6 mm². A rectangular outlet may be located on the top of the device or suitably is located on the end wall of the mixing chamber.

In other examples the outlet may be of circular cross-section (e.g. having a diameter of 0.4 to 1.2 mm, such as 0.6 to 1 mm, for example about 0.8 mm such as 0.8 mm). A circular outlet may suitably be located on the top of the device.

The total cross-sectional area of all outlets will suitably be less than 90% of the cross-sectional area of the mixing chamber, such as less than 80% and especially less than 70%. The total cross-sectional area of all outlets may be 20% or more of the cross-sectional area of the mixing chamber, such as 30% or more and especially 40% or more. The total cross-sectional area of all inlets may be suitably be 20 to 120% of the cross-sectional area of the mixing chamber, such as 25 to 110%, in particular about 100% or about 62.5%, such as 100% or 62.5% of the cross-sectional area of the mixing chamber.

Suitably the outer walls of the outer inlets are substantially continuous, such as continuous, with the sides of the mixing chamber.

It is advantageous for the microfluidic device to encompass a small area, such that multiple devices may be conveniently located on a single chip. Desirably the device, including connections for the inlets and outlets, is less than 100 mm in length, such as less than 80 mm, especially less than 60 mm and particularly less than 45 mm. Desirably each device, including connections for the inlets and outlets, is less than 20 mm in width, such as less than 10 mm, especially less than 7 mm and particularly less than 5 mm.

The microfluidic device may be formed from any suitable material, namely one which is tolerant of the components used in the first fluid (e.g. solution) and second fluid (e.g. solution) and which is amenable to manufacture. Suitable materials include silicon and glass. Stainless steel is another suitable material. Devices may be prepared from such materials by etching, e.g. silicon devices may be prepared by Deep Reactive Ion Etching (DRIE or plasma etching) and glass devices may be prepared by wet etching (HF etching). Chosen materials may be subjected to surface treatment to improve the characteristics of the surface.

To achieve a batch run duration which is a manageable time period (e.g. 240 minutes or less, especially 120 minutes or less) it is necessary for the system to achieve a sufficient level of productivity. Additionally, to aid batch to batch consistency by reducing the impact of startup and shutdown effects it is necessary for the run time to be of adequate length (e.g. at least 30 minutes, especially at least 60 minutes).

### Scale-Up

In order to facilitate production of liposomal adjuvant on an industrial scale (e.g. a scale of at least 0.5 g of lipid per minute, such as at least 1 g per minute, in particular at least 2 g per minute and especially at least 4 g per minute, such as a scale of at least 0.5 g of phosphatidylcholine lipid (e.g. DOPC) per minute, such as at least 1 g of phosphatidylcholine lipid (e.g. DOPC) per minute, in particular at least 2 g of phosphatidylcholine lipid (e.g. DOPC) per minute and especially at least 4 g of phosphatidylcholine lipid (e.g. DOPC) per minute), larger mixing chambers may be used or a plurality of mixing chambers (i.e. at least 2) may be operated in parallel. For example, 3 or more mixing chambers, in particular 4 or more, especially 8 or more, such as 16 or more (e.g. 16). The plurality of mixing chambers operated in parallel may be 128 or fewer, such as 64 or fewer, in particular 32 or fewer. Consequently, in some embodiments the plurality of mixing chambers is 2 to 128, such as 4 to 64, for example 8 to 32. Of particular interest is 12 to 20 mixing chambers, such as about 16, such as 16 mixing chambers.

In some circumstances each mixing chamber from the plurality of mixing chambers may be operated independently, with provision of the first fluid (e.g. solution) and second fluid (e.g. solution) to the mixing chamber by independent pumps (i.e. each pump not concurrently providing solution to any other mixing chamber). The first fluid (e.g. solution) and/or second fluid (e.g. solution) may be stored in independent containers (i.e. containers not concurrently providing first solution and/or second solution to more than one mixing chamber), or first fluid (e.g. solution) and/or second fluid (e.g. solution) may be stored in a container for use in more than one mixing chamber (such as all mixing chambers). Mixed material from each mixing chamber may be recovered individually and stored/processed, optionally being combined at a later stage, or may be combined (e.g. from all mixing chambers) before further processing and/or storage.

Conveniently some (such as all) mixing chambers in the plurality of mixing chambers are supplied by the same pumps. Conveniently mixed material from some (such as all) mixing chambers is collected before further processing and/or storage.

Suitably all mixing chambers in the plurality are substantially the same and/or suitably fluid flow within all mixing chambers is substantially the same, such that material obtained from each mixing chamber is substantially the same. Desirably in operation the flow rates measured in each mixing chamber of a plurality vary by less than 5% from the desired flow rate.

Optimally the mixing chambers, inlets and outlets, supply of first fluid (e.g. solution), second fluid (e.g. solution) and collection of mixed material of multiple mixing chambers are configured such that in operation they perform substantially identically.

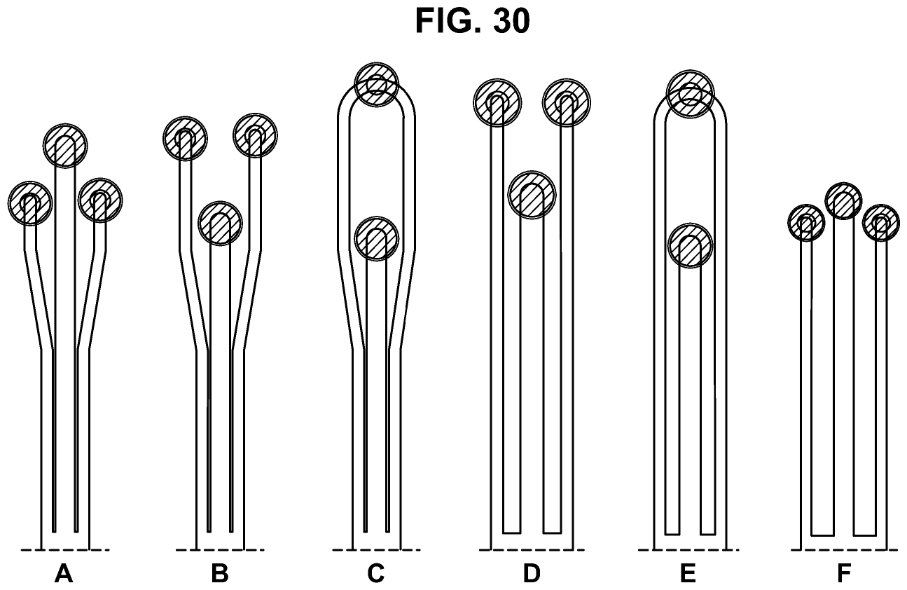

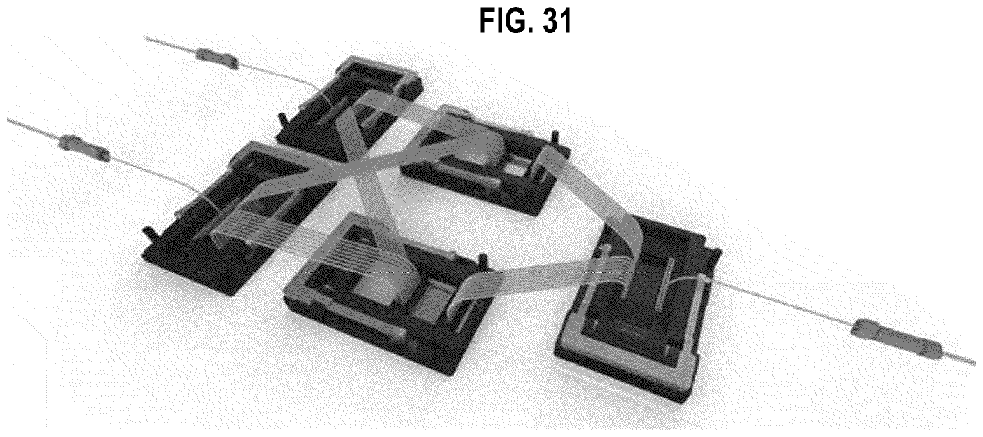

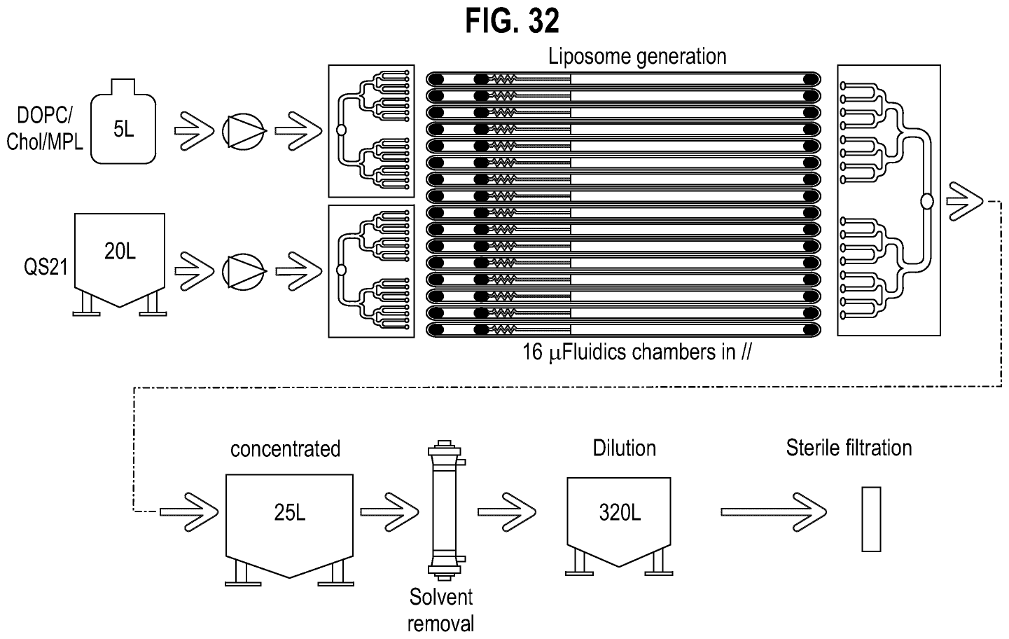

FIG. 31 illustrates an arrangement having a single connection for each of first and second fluids (left side of image), which are each split via inlet manifolds into 16 streams. 8 of the first and 8 of the second fluid streams are in turn connected to two 8 mixing chamber chips. The outlets from the 16 mixing chambers, 8 from each of two 8 mixing chamber chips, are then combined in a collection manifold to a single outlet connection (right side of image). FIG. 32 shows how such a 16 mixing chamber arrangement can be used in commercial scale operation (illustrating a flow rate ratio of 4:1).

Each mixing chamber from the plurality of mixing chambers may be configured as an individual chip or for convenience a number of mixing chambers may be combined in a single chip (e.g. up to 20 mixing chambers, such as 4 to 20, such as about 8 or about 16 mixing chambers, such as 8 or 16 mixing chambers). A number of such chips can be used in parallel to provide the plurality of chambers (e.g. two chips each of which contains about 8 mixing chambers, such as 8 mixing chambers, to provide a total of about 16 mixing chambers, such as 16 mixing chambers, to be operated in parallel).

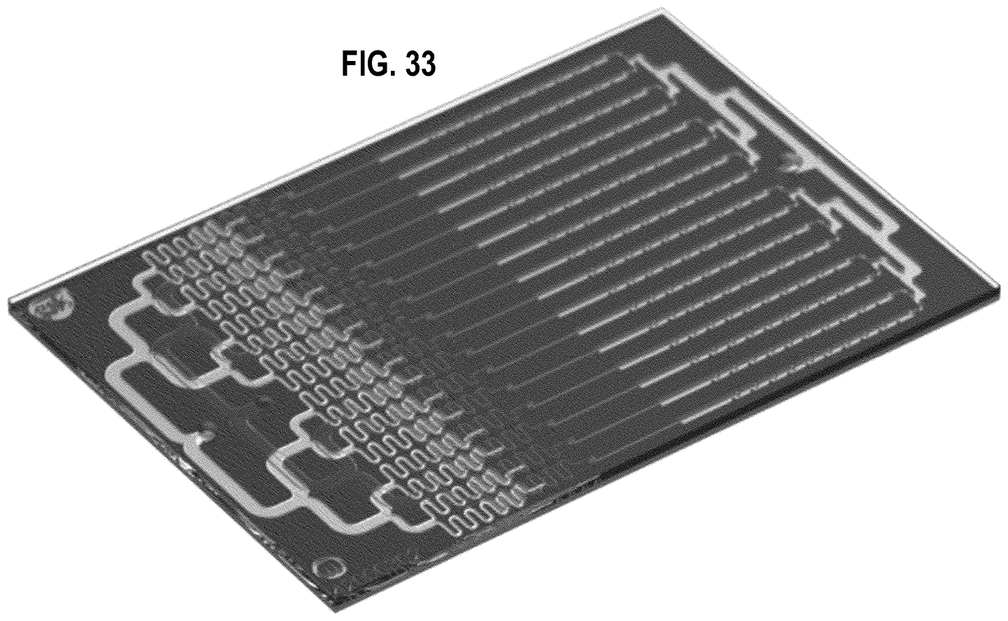

Suitably distribution manifolds for the first and second fluids or collection manifold(s) are incorporated into a multi-mixing chamber chip, such that each chip comprises a plurality of mixing chambers but has a single point of connection for the first fluid, second fluid or mixed material, suitably such integrated chips have a single point of connection for each of a first and a second fluid and a single point of connection for collection of mixed material. Such integrated chips may have 4 to 32 mixing chambers, such as 6 to 20 mixing chambers. Of particular interest is an integrated chip having 12 to 20 mixing chambers, such as about 16, such as 16 mixing chambers. Examples of such integrated chips are illustrated in FIG. 33 to FIG. 35.

Suitably the plurality of mixing chambers is capable of producing mixed material at a total flow rate of 50 to 2000 ml/min, such as 100 to 1000 ml/min, in particular 200 to 500 ml/min.

### Baffles

The devices of the present invention are characterised in particular in that the mixing chamber comprises one or more baffles. By the term baffle is meant a protrusion on the surface of the mixing chamber. Baffles may be located on the top, bottom or sides of the mixing chamber. Desirably baffles are located on the sides of the mixing chamber, such as on one side or on both sides. Typically, baffles are not present on the top or bottom of the mixing chamber.

The arrangement of a series of baffles, such as on one side of the mixing chamber, may be varied and they may for example be spaced substantially evenly (i.e. the distance between centroids of baffles is substantially consistent between neighbouring pairs along one side of the mixing chamber). Baffles may be spaced substantially evenly on both sides of the mixing chamber.

Baffles on at least one side (such as one side) of the mixing chamber may be spaced substantially unevenly (i.e. the distance between centroids of baffles is substantially inconsistent between neighbouring pairs along the mixing chamber). Baffles on both sides of the mixing chamber may be spaced substantially unevenly (i.e. the distance between centroids of baffles is substantially inconsistent between neighbouring pairs along one side of the mixing chamber).

Where baffles are present on both sides of the mixing chamber, these may be positioned substantially opposite each other. Alternatively, baffles on different sides of the mixing chamber may be positioned offset to each other, such as by 0.5 to 5 mm, such as 1 to 2.5 mm. Particular examples of offsets are about 1.75 mm such as 1.732 mm, about 1.26 mm such as 1.258 mm, about 1 mm, such as 1 mm and about 0.728 mm, such as 0.728 mm.

Where baffles are offset the offset before and after a baffle may be substantially the same (i.e. a baffle on one side is located substantially equidistant between two baffles on the opposite side) or the offset before and after a baffle may be substantially different. In certain device baffles are offset by 20 to 80% of the space between the baffles, especially 30 to 70%.

A mixing chamber will typically comprise a plurality of baffles, for example at least 4 baffles, such as at least 6 baffles, especially at least 8 baffles, in particular at least 10 baffles. A mixing chamber may comprise 100 or fewer baffles, such as 60 or fewer baffles, especially 40 or fewer baffles, in particular 25 or fewer baffles. Particular examples of numbers of baffles include about 12 or about 19, such as 12 or 19.

Baffles may be of any suitable shape, such as rectangular (e.g. square), wave, bell or trapezium shape, in particular trapezium. A plurality of baffles may be of the same or of different shapes, typically all baffles will be of the same shape.

Baffles may be of any suitable size. A plurality of baffles may be of substantially the same width (such as the same width) or may be of varied width, typically all baffles will be of substantially the same width (such as the same width). The term width is used to define the maximum distance by which a baffle protrudes from the surface of the mixing chamber acting to reduce the width of the mixing chamber.

The mixing chamber may comprise baffles 0.1 to 1 mm wide, such as 0.2 to 0.8 mm wide, especially 0.4 to 0.7 mm wide, in particular about 0.5 mm wide such as 0.5 mm wide.

The mixing chamber may comprise baffles 0.2 to 0.5 mm wide, such as 0.3 to 0.4 mm wide, in particular about 0.35 mm wide such as 0.35 mm wide.

The mixing chamber width at a baffle may be reduced by at least 10%, such as at least 20%, especially at least 25% and in particular at least 30%. The mixing chamber width at a baffle may be reduced by 80% or less, such as 60% or less, especially 50% or less and in particular 40% or less.

The mixing chamber width at a baffle may be reduced by 10 to 80%, such as 20 to 60%, especially to 50% (e.g. 35 to 50%) and in particular 30 to 40%.

Suitably the mixing chamber width at a baffle is 0.5 to 2 mm, such as 0.7 to 1.5 mm, especially 0.9 to 1.3 mm and in particular about 1.1 mm such as 1.1 mm.

The mixing chamber width at a baffle may be 0.4 to 0.9 mm, such as 0.5 to 0.8 mm and in particular about 0.65 mm such as 0.65 mm.

Suitably the mixing chamber comprises baffles on one side which are separated by 1 to 10 mm, such as 2 to 5 mm and in particular about 3.5 mm such as 3.464 mm. Desirably each baffle on one side of the mixing chamber is separated by 1 to 10 mm, such as 2 to 5 mm and in particular about 3.5 mm such as 3.464 mm.

Suitably the mixing chamber comprises baffles on two sides which are separated by 1 to 10 mm, such as 2 to 5 mm and in particular about 3.5 mm such as 3.464 mm. Desirably each baffle on each side of the mixing chamber is separated by 1 to 10 mm, such as 2 to 5 mm and in particular about 3.5 mm such as 3.464 mm.

The mixing chamber may comprise baffles on two sides which are separated by about 2.5 mm such as 2.516 mm. Desirably each baffle on each side of the mixing chamber is separated by about 2.5 mm such as 2.516 mm.

The mixing chamber may comprise baffles on two sides which are separated by about 2 mm such as 2 mm. Desirably each baffle on each side of the mixing chamber is separated by about 2 mm such as 2 mm.

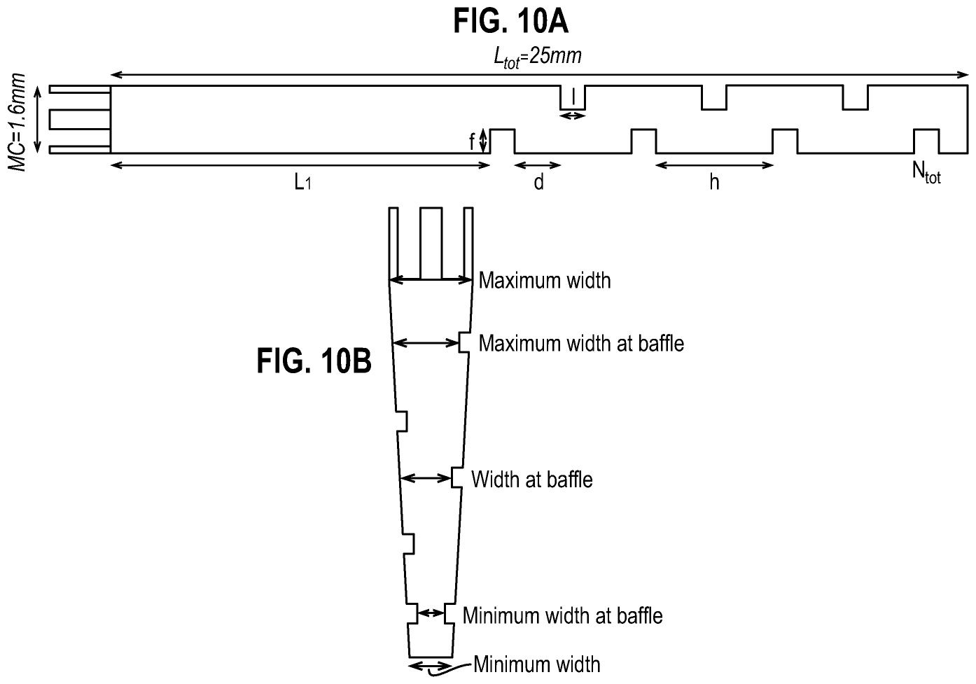

Baffles may be located at any suitable position along the mixing chamber. For example, the first baffle may be located 0.2 to 20 mm from the proximal end of the mixing chamber (illustrated by L₁ in FIG. 10 for a square profile baffle), such as 0.4 to 10 mm, especially 0.6 to 8 mm, in particular about 0.8 mm, about 4.4 mm or about 5.3 mm, such as 0.8, 4.4 or 5.3 mm.

Suitably the mixing chamber comprises baffles having a maximum length of 0.1 to 5 mm, such as 0.2 to 2 mm, especially 0.2 to 1 mm and in particular 0.25 to 0.7 mm, such as about 0.33 mm or about 0.55 mm, such as 0.33 mm or 0.55 mm. The term length is used to define the distance between the point at which a baffle begins to protrude at the proximal end to the point at which it ceases to protrude at the distal end (illustrated by I in FIG. 10 for a square profile baffle).

Suitably trapezium baffles have a minimum length of 3 mm or less, such as 1 mm or less, especially 0.5 mm or less and in particular 0.3 mm or less, such as about 0.15 mm or about 0.25 mm, such as 0.15 mm or 0.25 mm.

Suitably the maximum width of the mixing chamber at a baffle is 0.4 to 2 mm, such as 0.5 to 1.6 mm, especially 0.6 to 1.4 mm, in particular about 0.65 mm or about 1.1 mm, such as 0.65 mm or 1.1 mm.

Suitably the minimum width of the mixing chamber at a baffle is 0.4 to 2 mm, such as 0.5 to 1.6 mm, especially 0.6 to 1.4 mm, in particular about 0.65 mm or about 1.1 mm, such as 0.65 mm or 1.1 mm.

The maximum width of the mixing chamber and minimum width of the mixing chamber at a baffle may be the same the same (for example if the mixing chamber width and baffle width are constant or alternatively if the mixing camber width varied but the baffle width is also varied to compensate).

FIG. 10 illustrates some general features of microfluidic devices comprising baffles.

### PARTICULAR EMBODIMENTS

The present invention provides a microfluidic mixing device comprising:
a mixing chamber about 25 mm in length, having a substantially rectangular cross-section, substantially parallel sides spaced about 1.6 mm apart and substantially parallel top and bottom walls spaced to provide a depth of about 0.5 mm;
one inlet channel into the mixing chamber for a first fluid, being substantially centrally located at a proximal end of the mixing chamber, having a substantially rectangular cross-section, a width of about 0.27 mm and a depth of about 0.5 mm;
two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises about 19 baffles, the first baffle being located about 0.8 mm from the proximal end of the mixing chamber, baffles being separated by about 2.6 mm with an offset between the first and second sides of about 0.73 mm, the baffles being substantially trapezium in shape with a maximum length of about 0.55 mm, a minimum length of about 0.25 mm and a width of about 0.5 mm.

The present invention provides a microfluidic mixing device comprising:
a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises 17 to 21 baffles, the first baffle being located 0.72 to 0.88 mm from the proximal end of the mixing chamber, baffles being separated by 2.26 to 2.77 mm with an offset between the first and second sides of 0.656 to 0.8 mm, the baffles being substantially trapezium in shape with a maximum length of 0.495 to 0.605 mm, a minimum length of 0.225 to 0.275 mm and a width of 0.45 to 0.55 mm.

For example, a microfluidic mixing device comprising:
a mixing chamber 25 mm in length, having a rectangular cross-section, parallel sides spaced 1.6 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;

characterised in that the mixing chamber comprises 19 baffles, the first baffle being located 0.8 mm from the proximal end of the mixing chamber, baffles being separated by 2.516 mm with an offset between the first and second sides of 0.728 mm, the baffles being trapezium in shape with a maximum length of 0.55 mm, a minimum length of 0.25 mm and a width of 0.5 mm.

The present invention also provides microfluidic mixing device comprising:
a mixing chamber about 25 mm in length, having a substantially rectangular cross-section, substantially parallel sides spaced about 1.6 mm apart and substantially parallel top and bottom walls spaced to provide a depth of about 0.5 mm;
one inlet channel into the mixing chamber for a first fluid, being substantially centrally located at a proximal end of the mixing chamber, having a substantially rectangular cross-section, a width of about 0.27 mm and a depth of about 0.5 mm;
two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises about 19 baffles, the first baffle being located about 0.8 mm from the proximal end of the mixing chamber, baffles being separated by about 2.6 mm with an offset between the first and second sides of about 1.3 mm, the baffles being substantially trapezium in shape with a maximum length of about 0.55 mm, a minimum length of about 0.25 mm and a width of about 0.5 mm.

The present invention also provides microfluidic mixing device comprising:
a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises 17 to 21 baffles, the first baffle being located 0.72 to 0.88 mm from the proximal end of the mixing chamber, baffles being separated by 2.26 to 2.77 mm with an offset between the first and second sides of 1.13 to 1.38 mm, the baffles being substantially trapezium in shape with a maximum length of 0.495 to 0.605 mm, a minimum length of 0.225 to 0.275 mm and a width of 0.45 to 0.55 mm.

For example, a microfluidic mixing device comprising:
a mixing chamber 25 mm in length, having a rectangular cross-section, parallel sides spaced 1.6 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;

characterised in that the mixing chamber comprises 19 baffles, the first baffle being located 0.8 mm from the proximal end of the mixing chamber, baffles being separated by 2.516 mm with an offset between the first and second sides of 1.258 mm, the baffles being trapezium in shape with a maximum length of 0.55 mm, a minimum length of 0.25 mm and a width of 0.5 mm.

Also provided is a microfluidic mixing device comprising:
a mixing chamber about 25 mm in length, having a substantially rectangular cross-section, substantially parallel sides spaced about 1.6 mm apart and substantially parallel top and bottom walls spaced to provide a depth of about 0.5 mm;
one inlet channel into the mixing chamber for a first fluid, being substantially centrally located at a proximal end of the mixing chamber, having a substantially rectangular cross-section, a width of about 0.27 mm and a depth of about 0.5 mm;
two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises about 12 baffles, the first baffle being located about 4.4 mm from the proximal end of the mixing chamber, baffles being separated by about 3.5 mm with an offset between the first and second sides of about 1.7 mm, the baffles being substantially trapezium in shape with a maximum length of about 0.55 mm, a minimum length of about 0.25 mm and a width of about 0.5 mm.

Also provided is a microfluidic mixing device comprising:
a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises 10 to 14 baffles, the first baffle being located 3.96 to 4.84 mm from the proximal end of the mixing chamber, baffles being separated by 3.12 to 3.81 mm with an offset between the first and second sides of 1.56 to 1.91 mm, the baffles being substantially trapezium in shape with a maximum length of 0.495 to 0.605 mm, a minimum length of 0.225 to 0.275 mm and a width of 0.45 to 0.55 mm.

For example, a microfluidic mixing comprising:
a mixing chamber 25 mm in length, having a rectangular cross-section, parallel sides spaced 1.6 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;

characterised in that the mixing chamber comprises 12 baffles, the first baffle being located 4.4 mm from the proximal end of the mixing chamber, baffles being separated by 3.464 mm with an offset between the first and second sides of 1.732 mm, the baffles being trapezium in shape with a maximum length of 0.55 mm, a minimum length of 0.25 mm and a width of 0.5 mm.

Further provided is a microfluidic mixing device according to any of the preceding claims comprising:
a mixing chamber about 25 mm in length, having a substantially rectangular cross-section, substantially parallel sides spaced about 1 mm apart and substantially parallel top and bottom walls spaced to provide a depth of about 0.5 mm;
one inlet channel into the mixing chamber for a first fluid, being substantially centrally located at a proximal end of the mixing chamber, having a substantially rectangular cross-section, a width of about 0.27 mm and a depth of about 0.5 mm;
two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises about 19 baffles, the first baffle being located about 5.3 mm from the proximal end of the mixing chamber, baffles being separated by about 2 mm with an offset between the first and second sides of about 1 mm, the baffles being substantially trapezium in shape with a maximum length of about 0.33 mm, a minimum length of about 0.15 mm and a width of about 0.35 mm.

Further provided is a microfluidic mixing device according to any of the preceding claims comprising:
a mixing chamber 22.5 to 27.5 mm in length, having a substantially rectangular cross-section, substantially parallel sides spaced 0.9 to 1.1 mm apart and substantially parallel top and bottom walls spaced to provide a depth of 0.45 to 0.55 mm;
one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises 17 to 21 baffles, the first baffle being located 4.77 to 5.83 mm from the proximal end of the mixing chamber, baffles being separated by 1.8 to 2.2 mm with an offset between the first and second sides of 0.9 to 1.1 mm, the baffles being substantially trapezium in shape with a maximum length of 0.297 to 0.363 mm, a minimum length of 0.135 to 0.165 mm and a width of 0.315 to 0.385 mm.

For example, a microfluidic mixing device comprising:
a mixing chamber 25 mm in length, having a rectangular cross-section, parallel sides spaced 1 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;

characterised in that the mixing chamber comprises 19 baffles, the first baffle being located 5.3 mm from the proximal end of the mixing chamber, baffles being separated by 2 mm with an offset between the first and second sides of 1 mm, the baffles being trapezium in shape with a maximum length of 0.33 mm, a minimum length of 0.15 mm and a width of 0.35 mm.

Also provided is a chip comprising a plurality of microfluidic mixing devices (such as 4 to 20, especially 16), each device comprising:
a mixing chamber about 25 mm in length, having a substantially rectangular cross-section, substantially parallel sides spaced about 1.6 mm apart and substantially parallel top and bottom walls spaced to provide a depth of about 0.5 mm;
one inlet channel into the mixing chamber for a first fluid, being substantially centrally located at a proximal end of the mixing chamber, having a substantially rectangular cross-section, a width of about 0.27 mm and a depth of about 0.5 mm;
two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises about 12 baffles, the first baffle being located about 4.4 mm from the proximal end of the mixing chamber, baffles being separated by about 3.5 mm with an offset between the first and second sides of about 1.7 mm, the baffles being substantially trapezium in shape with a maximum length of about 0.55 mm, a minimum length of about 0.25 mm and a width of about 0.5 mm.

Also provided is a chip comprising a plurality of microfluidic mixing devices (such as 4 to 20, especially 16), each device comprising:
a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises 10 to 14 baffles, the first baffle being located 3.96 to 4.84 mm from the proximal end of the mixing chamber, baffles being separated by 3.12 to 3.81 mm with an offset between the first and second sides of 1.56 to 1.91 mm, the baffles being substantially trapezium in shape with a maximum length of 0.495 to 0.605 mm, a minimum length of 0.225 to 0.275 mm and a width of 0.45 to 0.55 mm.

For example, a chip comprising a plurality of microfluidic mixing devices (such as 4 to 20, especially 16), each device comprising:
a mixing chamber 25 mm in length, having a rectangular cross-section, parallel sides spaced 1.6 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;

characterised in that the mixing chamber comprises 12 baffles, the first baffle being located 4.4 mm from the proximal end of the mixing chamber, baffles being separated by 3.464 mm with an offset between the first and second sides of 1.732 mm, the baffles being trapezium in shape with a maximum length of 0.55 mm, a minimum length of 0.25 mm and a width of 0.5 mm.

Also provided is an integrated chip comprising a plurality of microfluidic mixing devices (such as 4 to 20, especially 6 to 18, in particular 16), the integrated chip having a single point of connection for each of a first and a second fluid and a single point of connection for collection of mixed material, each device comprising:
a mixing chamber about 25 mm in length, having a substantially rectangular cross-section, substantially parallel sides spaced about 1.6 mm apart and substantially parallel top and bottom walls spaced to provide a depth of about 0.5 mm;
one inlet channel into the mixing chamber for a first fluid, being substantially centrally located at a proximal end of the mixing chamber, having a substantially rectangular cross-section, a width of about 0.27 mm and a depth of about 0.5 mm;
two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises about 12 baffles, the first baffle being located about 4.4 mm from the proximal end of the mixing chamber, baffles being separated by about 3.5 mm with an offset between the first and second sides of about 1.7 mm, the baffles being substantially trapezium in shape with a maximum length of about 0.55 mm, a minimum length of about 0.25 mm and a width of about 0.5 mm.

Also provided is an integrated chip comprising a plurality of microfluidic mixing devices (such as 4 to 20, especially 6 to 18, in particular 16), the integrated chip having a single point of connection for each of a first and a second fluid and a single point of connection for collection of mixed material, each device comprising:
a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
said inlet channels being disposed substantially symmetrically at the proximal end of the mixing chamber and wherein the direction of flow from the inlet channels into the mixing chamber is substantially parallel to the general direction of flow in the mixing chamber;
one outlet for mixed material at a distal end of the mixing chamber;

characterised in that the mixing chamber comprises 10 to 14 baffles, the first baffle being located 3.96 to 4.84 mm from the proximal end of the mixing chamber, baffles being separated by 3.12 to 3.81 mm with an offset between the first and second sides of 1.56 to 1.91 mm, the baffles being substantially trapezium in shape with a maximum length of 0.495 to 0.605 mm, a minimum length of 0.225 to 0.275 mm and a width of 0.45 to 0.55 mm.

For example, an integrated chip comprising a plurality of microfluidic mixing devices (such as 4 to 20, especially 6 to 18, in particular 16), the integrated chip having a single point of connection for each of a first and a second fluid and a single point of connection for collection of mixed material, each device comprising:
a mixing chamber 25 mm in length, having a rectangular cross-section, parallel sides spaced 1.6 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;

characterised in that the mixing chamber comprises 12 baffles, the first baffle being located 4.4 mm from the proximal end of the mixing chamber, baffles being separated by 3.464 mm with an offset between the first and second sides of 1.732 mm, the baffles being trapezium in shape with a maximum length of 0.55 mm, a minimum length of 0.25 mm and a width of 0.5 mm.

### Methods

The devices may be used for a range of purposes. Of particular interest is the use of the devices in the manufacture of liposomal adjuvants. Consequently, there is provided a method of manufacturing a liposomal adjuvant using a microfluidic device as described herein, comprising the following steps:
(a) mixing in the device a first solution comprising a solvent and a lipid, and a second solution comprising water; and
(b) removing the solvent.

Also provided is a method of manufacturing a liposomal adjuvant comprising the following steps:
(a) mixing in the device a first solution comprising a solvent and lipid, and a second solution comprising water;
(b) adding a saponin; and
(c) removing the solvent.

Additionally provided is a method of manufacturing a liposomal adjuvant comprising the following steps:
(a) mixing in the device a first solution comprising a solvent and lipid, and a second solution comprising water;
(b) removing the solvent; and
(c) adding a saponin.

Further provided is a method of manufacturing a liposomal adjuvant comprising the following steps:
(a) mixing in the device a first solution comprising a solvent and lipid, and a second solution comprising water;
(b) adding a TLR4 agonist; and
(c) removing the solvent.

Also provided is a method of manufacturing a liposomal adjuvant comprising the following steps:
(a) mixing in the device a first solution comprising a solvent and lipid, and a second solution comprising water;
(b) removing the solvent; and
(c) adding a TLR4 agonist.

The present invention provides a method of manufacturing a liposomal concentrate of use in preparing a liposomal adjuvant using a microfluidic device, comprising the step of mixing in the device a first solution comprising a solvent and lipid, and a second solution comprising water.

Also provided is a method of manufacturing a liposomal concentrate of use in the preparation of a liposomal adjuvant using a microfluidic device, comprising the following steps:
(a) mixing in the device a first solution comprising a solvent and lipid, and a second solution comprising water; and
(b) adding a TLR4 agonist.

Additionally provided is a method of manufacturing a liposomal concentrate of use in the preparation of a liposomal adjuvant using a microfluidic device comprising the following steps:
(a) mixing in the device a first solution comprising a solvent and lipid, and a second solution comprising water; and
(b) adding a saponin.

Further provided is a method of manufacturing a liposomal concentrate of use in preparing a liposomal adjuvant using a microfluidic device comprising the following steps:
(a) mixing in the device a first solution comprising a solvent and lipid, and a second solution comprising water;
(b) adding a saponin; and
(c) adding a TLR4 agonist;

wherein steps (b) and (c) may be in either order, or may be performed in a single step.

Also provided are a liposomal adjuvant and a liposomal concentrate obtainable from, such as obtained from, the methods described herein.

In certain methods, a TLR4 agonist may be added to the recovered mixed material before removal of the solvent. In other methods a TLR4 agonist may be added after removal of the solvent (in such circumstances the amount of TLR4 will typically be equivalent to the amounts which would be used if added earlier).

### First Solution

The first solution (the ‘organic’ phase) typically comprises solvent and lipid. Suitably the lipid is a phosphatidylcholine. Desirably the first solution comprises a sterol. Suitably the first solution comprises a solvent, DOPC and a sterol.

The solvent should solubilise the lipid, such as phosphatidylcholine (such as DOPC), and any other component (such as sterol) present to provide the first solution as a single phase.

Furthermore, the solvent should be miscible with the aqueous solution, such that mixing of the first solution and second solution results in a single liquid phase which comprises a suspension of liposomes.

The solvent will be an organic solvent or a single-phase mixture comprising at least one organic solvent.

The solvent may comprise a short chain organic alcohol, such as ethanol and/or isopropanol.

Suitably, the solvent will comprise ethanol, such as at a concentration of between 70 to 90% v/v, more suitably between 75 to 85% v/v, or between 78 to 82% v/v.

Suitably, the solvent will comprise isopropanol, such as at a concentration of between 10 to 30% v/v, more suitably between 15 to 25% v/v, or between 18 to 22% v/v.

Suitably, the solvent will consist essentially of ethanol at a concentration of between 70 to 90% v/v and isopropanol at a concentration of between 10 to 30% v/v, such as ethanol at a concentration of between 75 to 85% v/v and isopropanol at a concentration of between 15 to 25% v/v, especially ethanol at a concentration of between 78 to 82% v/v and isopropanol at a concentration of between 18 to 22% v/v, in particular ethanol at a concentration of 80% v/v and isopropanol at a concentration of 20% v/v. At higher ethanol concentrations, such as above 90% v/v ethanol, the solubilising capacity of the solvent is limited (which ultimately constrains system capacity). At lower ethanol concentrations, such as below 70% v/v ethanol, the process may be more sensitive to operating parameters, such as temperature.

Lipids of use in the present invention will typically be membrane forming lipids. Membrane forming lipids comprise a diverse range of structures including phospholipids (for example phosphatidyl choline, phosphatidyl ethanolamine, phosphatidyl glycerol, phosphatidyl inositol and phosphatidyl serine), ceramides and sphingomyelins. Membrane forming lipids typically have a polar head group (which in a membrane aligns towards the aqueous phase) and one or more (e.g. two) hydrophobic tail groups (which in a membrane associate to form a hydrophobic core). The hydrophobic tail groups will typically be in the form of acyl esters, which may vary both in their length (for example from 8 to 26 carbon atoms) and their degree of unsaturation (for example one, two or three double bonds).

Lipids of use in the present invention may be of natural or synthetic origin, and may be a single pure component (e.g. 90% pure, especially 95% pure and suitably 99% pure on a weight basis), a single class of lipid components (for example a mixture of phosphatidyl cholines, or alternatively, a mixture of lipids with a conserved acyl chain type) or may be a mixture of many different lipid types.

In one embodiment of the invention the lipid is a single pure component.

Pure lipids are generally of synthetic or semi-synthetic origin. Examples of pure lipids of use in the present invention include phosphatidyl cholines (for example, DLPC, DMPC, DPPC, DSPC and DOPC; in particular DLPC, DMPC, DPPC and DOPC; especially DOPC) and phosphatidyl glycerols (for example DPPG), suitably phosphatidyl cholines. The use of pure lipids is desirable due to their defined composition, however, they are generally more expensive.

In one embodiment of the invention the lipid is a mixture of components.

Mixtures of lipids of use in the present invention may be of natural origin, obtained by extraction and purification by means known to those skilled in the art. Lipid mixtures of natural origin are generally significantly cheaper than pure synthetic lipids. Naturally derived lipids include lipid extracts from egg or soy, which extracts will generally contain lipids with a mixture of acyl chain lengths, degrees of unsaturation and headgroup types. Lipid extracts of plant origin may typically be expected to demonstrate higher levels of unsaturation than those of animal origin. It should be noted that, due to variation in the source, the composition of lipid extracts may vary from batch to batch.

In one embodiment of the invention the lipid is a lipid extract containing at least 50%, especially at least 75% and suitably at least 90% by weight of phospholipids of a single headgroup type (e.g. phosphatidyl cholines). In a second embodiment of the invention particular lipid extracts may be preferred due to their relatively cheap cost. In a third embodiment of the invention the lipid is a lipid mixture having a conserved acyl chain length (e.g. at least 50%, especially at least 75% and suitably at least 90% by weight), for example 12 (e.g. lauryl), 14 (e.g. myristyl), 16 (e.g. palmityl) or 18 (e.g. stearyl or oleoyl) carbons atoms in length.

Suitably, a lipid extract of use in the present invention will comprise at least 50% phospholipids by weight (for example, phosphatidyl cholines and phosphatidyl ethanolamines), especially at least 55% phospholipids by weight, in particular at least 60% phospholipids by weight (such as 75% or 90%).

Lipid mixtures may also be prepared by the combination of pure lipids, or by the combination of one lipid extract with either other lipid extracts or with pure lipids.

Certain lipids of interest include DOTAP (1,2-dioleoyl-3-trimethylammonium-propane) and DDA (dimethyldioctadecylammonium). Combinations of DMPC and DOTAP are of interest.

As mentioned, the first solution will desirably comprise a phosphatidylcholine. The phosphatidylcholine will contain unbranched acyl chains having 12 to 20 carbon atoms, optionally with one double bond, of particular interest are those with acyl chains having 14 to 18 carbon atoms, optionally with one double bond. Typically, each of the two acyl chains in a lipid molecule are identical. Particular phosphatidylcholine lipids of interest include: the saturated phosphatidylcholine lipids—dilauroyl phosphatidylcholine (DLPC), dimyristoyl phosphatidylcholine (DMPC), dipalmitoyl phosphatidylcholine (DPPC), distearoyl phosphatidylcholine (DSPC) and diarachidoyl phosphatidylcholine (DAPC); and unsaturated phosphatidylcholine lipids dipalmitoleoyl phosphatidylcholine and dioleoyl phosphatidylcholine (DOPC); and mixtures thereof. Suitably the phosphatidylcholine lipid is substantially purified from other lipids. Typically the phosphatidylcholine lipid is at least 80% pure, such as at least 90% pure, especially at least 95% pure, in particular 98% pure, for example at least 99% or even at least 99.8% pure by weight.

Of particular use in the invention are first solutions comprising a solvent and 100 to 170 mg/ml lipid, wherein the solvent comprises 70 to 90% v/v ethanol and 10 to 30% v/v isopropyl alcohol. Suitably the lipid is phosphatidylcholine.

As mentioned, the first solution suitably comprises DOPC (dioleoyl phosphatidylcholine). Suitably the DOPC is substantially purified from other lipids, both of other acyl chain types and other headgroup types. Typically the DOPC is at least 90% pure, such as at least 95% pure, especially at least 98% pure, in particular 99% pure, for example at least 99.8% pure by weight.

Suitably the first solution comprises 100 to 170 mg/ml DOPC, such as 100 to 160 mg/ml DOPC, especially 120 to 160 mg/ml. The first solution may comprise 120 to 150 mg/ml DOPC, such as 120 to 140 mg/ml DOPC. In particular, the first solution may comprise around 130 mg/ml DOPC (e.g. 125 to 135 mg/ml DOPC, especially 130 mg/ml DOPC).

The sterol will typically be cholesterol. Cholesterol is disclosed in the Merck Index, 13th Edn., page 381, as a naturally occurring sterol found in animal fat. Cholesterol has the formula (C₂₇H₄₆O) and is also known as (3β)-cholest-5-en-3-ol.

Suitably the first solution comprises 20 to 50 mg/ml sterol (e.g. cholesterol), such as 25 to 40 mg/ml, especially around 32.5 mg/ml (e.g. 30 to 35 mg/ml, in particular 32.5 mg/ml).

Suitably the dry weight of the first solution is 100 to 250 mg/ml, such as 140 to 220 mg/ml, especially 150 to 220 mg/ml.

The invention therefore may utilise a first solution comprising a solvent and 100 to 170 mg/ml lipid, wherein the solvent comprises 70 to 90% v/v ethanol and 10 to 30% v/v isopropyl alcohol. Suitably the lipid is DOPC.

The ratio of lipid (e.g. phosphatidylcholine, such as DOPC) to sterol is usually 3:1 to 5:1 w/w, such as 3.5:1 to 4.5:1 w/w.

In some embodiments the first solution consists essentially of a solvent and 100 to 160 mg/ml lipid and 30 to 40 mg/ml cholesterol wherein the solvent comprises 70 to 90% v/v ethanol and to 30% v/v isopropyl alcohol. Desirably the lipid is phosphatidylcholine. Suitably the lipid is DOPC.

In order to prepare liposomal adjuvants comprising a TLR4 agonist, the TLR4 agonist may optionally be included in the first solution, in particular a lipopolysaccharide, such as a monophosphoryl lipid A such as 3D-MPL. The first solution may contain 1 to 25 mg/ml of the TLR4 agonist, such as 2 to 16 mg/ml, especially 3 to 12 mg/ml and in particular 4 to 10 mg/ml (e.g. around 6.5 mg/ml, such as 5.5 to 7.5 mg/ml, especially 6.5 mg/ml).

Other features of the method may be as described for the first solution, e.g. the solution comprises 100 to 160 mg/ml lipid and 30 to 40 mg/ml cholesterol and wherein the solvent comprises 70 to 90% v/v ethanol and 10 to 30% v/v isopropyl alcohol. Desirably the lipid is phosphatidylcholine. Suitably the lipid is DOPC. Suitably the solution comprises 4 to 10 mg/ml TLR4 agonist, in particular lipopolysaccharide, such as a monophosphoryl lipid A, such as 3D-MPL.

Suitably the invention provides a solution consisting essentially of 100 to 160 mg/ml lipid and to 40 mg/ml cholesterol and wherein the solvent comprises 70 to 90% v/v ethanol and 10 to 30% v/v isopropyl alcohol. Desirably the lipid is phosphatidylcholine, more suitably the lipid is DOPC. Suitably the solution comprises 4 to 10 mg/ml TLR4 agonist, in particular lipopolysaccharide, such as a monophosphoryl lipid A, such as 3D-MPL.

### Second Solution

The second solution (the ‘aqueous’ phase) typically comprises water and in some methods may also comprise a saponin. The second solution may consist of water.

The second solution acts as a counter solvent, causing the formation of liposomes on mixing with the first solution. The faster the precipitation of components from the first solution, typically the smaller the liposomes obtained.

The second solution will be substantially aqueous and will comprise at least 90% water v/v, such as at least 95% water, especially at least 98% water, in particular at least 99% water such as 100% water.

When present in the second solution, suitably the saponin is present at a concentration of 0.05 to 25 mg/ml, such as 0.2 to 10 mg/ml, especially 0.5 to 5 mg/ml and in particular 0.8 to 3 mg/ml. The saponin may be present at a concentration of about 1.625 mg/ml, such as 1.2 to 2 mg/ml, especially 1.625 mg/ml, particularly for a ratio of flow rates for the first and second solutions the range 1:4, such as 1:4. Alternatively, the saponin may be present at a concentration of about 2.167 mg/ml, such as 1.6 to 2.6 mg/ml, especially 2.167 mg/ml, particularly fora ratio of flow rates for the first and second solutions the range 1:3, such as 1:3.

When the saponin is not present in the second solution, suitably the second solution consists essentially of (such as consists of) water.

When the saponin is present in the second solution, suitably the second solution consists essentially of (such as consists of) water and saponin, for example the second solution may be saponin (such as QS-21) in water for injection.

The ionic strength of the second solution will suitably be 150 nM or lower, such as 100 nM or lower, in particular 80 nM or lower, especially 60 nM or lower, for example 40 nM or lower.

Conductivity may be a convenient surrogate for the ionic strength of an aqueous solution. The conductivity of the second solution will suitably be 12 mS/cm or lower, for example 10 mS/cm or lower, 8 mS/cm or lower, 6 mS/cm or lower, or 4 mS/cm or lower.

Suitably, the second solution consists essentially of aqueous saponin.

### Microfluidic Operation

Optimal operating conditions will depend on the precise configuration of the device and the desired characteristics of the product.

Suitably, the total flow rate into the mixing chamber is greater than 8 ml/min/mm² for example 12 to 40 ml/min/mm² of mixing chamber cross-section. The total flow rate into the mixing chamber, may be 16 to 28 ml/min/mm², especially 17.5 to 25 ml/min/mm² and in particular 19 to 21 (e.g. 20 ml/min/mm²). The total flow rate into the mixing chamber may be 26 to 38 ml/min/mm², especially 28 to 36 ml/min/mm² and in particular 30 to 34 (e.g. 32 ml/min/mm²). The total flow rate into the mixing chamber may be 10 to 25 ml/min/mm², especially 12.5 to 22.5 ml/min/mm² and in particular 15 to 20 (e.g. 17.5 ml/min/mm²).

Suitably the ratio of flow rates for the first and second solutions will be in the range of 1:2 to 1:6, such as 1:3 to 1:5, especially 1:3.5 to 1:4.5 and in particular 1:4. The ratio of flow rates for the first and second solutions may be 1:2.5 to 1:3.5, such as 1:3. High levels of solvent in mixed material may impact the stability of liposomes so ratios of flow rates which result in high solvent concentrations are desirably avoided—solvent concentrations of 50% result from a ratio of 1:1, 33% for 1:2, 25% for ratio 1:3, 20% for ratio 1:4 and 16.6% for ratio 1:5. Low flow rate of the first solution reduces system productivity. Ratios of flow rates which result in relatively large volumes of mixed material are less desirable due to the safety protocols associated with the handling and use of solvent containing compositions which exceed certain thresholds (e.g. 50 L).

Suitably, the flow rate of the first solution into the mixing chamber is in the range of 2 to 10 ml/min/mm² of mixing chamber cross-section. The flow rate of the first solution into the mixing chamber may be 2 to 6 ml/min/mm², especially 3.5 to 5.5 ml/min/mm² and in particular 3 to 5 (e.g. 4) ml/min/mm². The flow rate of the first solution into the mixing chamber may be 4.35 ml/min/mm². The flow rate of the first solution into the mixing chamber may alternatively be 4.4 to 8.4 ml/min/mm², especially 4.9 to 6.9 ml/min/mm² and in particular 5.4 to 7.4 (e.g. 6.4) ml/min/mm².

Suitably, the flow rate of the second solution into the mixing chamber is in the range of 11 to 35 ml/min/mm² of mixing chamber cross-section. The flow rate of the second solution into the mixing chamber may be 12 to 20 ml/min/mm², especially 14 to 18 ml/min/mm² and in particular 15 to 17 (e.g. 16) ml/min/mm². The flow rate of the second solution into the mixing chamber may be 10 to 16 ml/min/mm², especially 11 to 15 ml/min/mm² and in particular 12 to 14 (e.g. 13.125) ml/min/mm².

The flow rate of the second solution into the mixing chamber may alternatively be 21.6 to 29.6 ml/min/mm², especially 23.6 to 27.6 ml/min/mm² and in particular 24.6 to 26.6 (e.g. 25.6) ml/min/mm².

The first solution and second solution will typically be provided at a temperature in the region of 10 to 30° C., such as 15 to 25° C., in particular 18 to 22° C. especially 20° C.), and may be at the same or different temperatures, suitably at the same temperature and especially at 20° C.

The mixing chamber may be maintained at a temperature in the region of 10 to 30° C., such as 15 to 25° C., in particular 18 to 22° C., especially 20° C. Dependent on the design of the device and environmental conditions it may only be necessary to actively control the temperature of the first solution and second solution, and not to actively control the mixing chamber temperature. The mixing of the first solution and second solution may be mildly exothermic. Lower operating temperatures result in the formation of smaller liposomes.

The microfluidic device may be operated within a controlled temperature environment, e.g. where the temperature is maintained in the range of 10 to 30° C., such as 15 to 25° C., in particular about 20° C. (such as 18 to 22° C., in particular 20° C.).

The operating pressure of the system need not be controlled.

Suitably, the maximum Reynolds number within the mixing chamber is 2100, in particular 1800, such as 1500, especially 1000, for example 600. The maximum Reynolds number within the mixing chamber is suitably within the range of 25 to 1500, more suitably between 50 to 1000, in particular 100 to 600 and especially 150 to 500. Methods for calculating the Reynolds number are known to those skilled in the art and are illustrated in the examples herein.

### Liposomes

Upon mixing of the first solution and second solution, liposomes will form.

The term ‘liposome’ is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds.

Liposome size may vary from 30 nm to several um depending on the phospholipid composition and the method used for their preparation.

The liposomes of the present invention may contain phosphatidylcholine lipid, or, consist essentially of phosphatidylcholine lipid (with saponin, TLR4 agonist and sterol agonist as applicable).

Suitably the liposomes of the present invention contain DOPC, or, consist essentially of DOPC and sterol (with saponin and TLR4 agonist as applicable).

In the present invention, the liposome size will be in the range of 50 nm to 200 nm, especially 60 nm to 180 nm, such as 70 to 165 nm. Optimally, the liposomes should be stable and have a diameter of approximately 100 nm to allow convenient sterilization by filtration. Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. Suitably the average particle size is between 95 and 120 nm, and/or, the polydispersity (PdI) index is not more than 0.35, in particular not more than 0.3, such as not more than 0.25. In one embodiment the average particle size is between 95 and 120 nm, and/or, the polydispersity (PdI) index is not more than 0.2.

The average particle size may be 90 to 120 nm, and/or, the polydispersity (PdI) index is not more than 0.35, in particular not more than 0.3, such as not more than 0.25. In one embodiment the average particle size is 90 to 120 nm, and/or, the polydispersity (PdI) index is not more than 0.2.

In some circumstances the presence of solvents and certain additional components can impact the liposome size. Consequently, the liposome size is suitably measured after solvent removal and the incorporation of any additional components.

### Removing the Solvent

The recovered mixed material will comprise liposomes in water and solvent. Such material is a liposomal concentrate of use in preparing a liposomal adjuvant, said liposomal concentrate comprising water, a solvent and lipid, optionally with a saponin, TLR4 agonist and sterol (e.g. cholesterol), such as comprising water, a solvent, DOPC, saponin, TLR4 agonist and cholesterol. The recovered material may be stored for later use or may be further processed to remove some or all of the solvent.

To facilitate use of the liposomes in an adjuvant it is desirable to remove substantially all organic solvent (e.g. leaving at least 98% water w/w, such as at least 99% water, especially at least 99.5% water, in particular at least 99.9% water such as at least 99.99%).

Suitably the residual organic solvent is at a level which equates to less than 150 ug per human dose, such as less than 100 ug per human dose, such as less than 50 ug per human dose and especially less than 20 ug per human dose (e.g. 10 ug or less per human dose). Desirably the residual organic solvent is at a level which is compliant with International Council For Harmonisation Of Technical Requirements For Pharmaceuticals For Human Use Guideline For Residual Solvents Q3C(R6).

Solvent removal may be performed by a range of methods, which may be used individually or in combination. Suitable methods include ultrafiltration and dialysis, especially diafiltration.

The removal of at least a portion of the solvent, such as substantially all of the solvent, can be performed by dialysis. Dialysis is the use of semi-permeable containment vessel that is selectively permeable such that solvent will pass through the semi-permeable portion of the vessel and liposomes (also saponin and TLR4 agonist if present) will be retained when recovered material is introduced to the semi-permeable containment vessel. For example, the semi-permeable containment vessel used can include a single semi-permeable membrane and solvent removal can be achieved by immersing the semi-permeable containment vessel comprising the recovered material in an exchange medium and allowing the liquids separated by the membrane to reach equilibrium by diffusion. Dialysis may be undertaken in batch or continuous modes of operation. For example, dialysis can be repeated multiple times with batch replacement of the exchange medium to achieve a desired level of solvent removal. Dialysis can also be in a continuous process where the recovered material and/or exchange medium is continuously undergoing replacement. Exemplary dialysis membranes which may be of use in the present methods include 7 kDa membranes

The removal of at least a portion of the solvent, such as substantially all of the solvent, can be performed by ultrafiltration. Ultrafiltration is the use of a containment vessel including a first compartment and a second compartment separated by a semi-permeable membrane. The recovered material can be placed into the first compartment of the containment vessel which can then be subjected to a positive pressure relative to the second compartment such that liquid is forced across the semi-permeable portion of the containment vessel. Diafiltration is a form of ultrafiltration wherein at least a portion of the remaining liquid can be replaced with an exchange medium by addition of the exchange medium to the first compartment of the vessel. Consequently, as the ultrafiltration progresses, the remaining liquid will tend towards the composition of the exchange medium. Diafiltration can be undertaken in a range of ways—continuous (also known as constant volume) wherein exchange medium is added at a comparable rate to liquid filtration over the membrane; discontinuous, wherein the volume of the remaining liquid varies and exchange medium is added in a discontinuous manner (e.g. by initial dilution and subsequent concentration to original volume or by initial concentration and subsequent dilution to original volume or the like). The optimal operating mode may depend on a number of factors including: 1) initial sample volume, concentration and viscosity 2) required final sample concentration 3) stability of sample at various concentrations 4) volume of buffer required for diafiltration 5) total processing time 6) reservoir size available 7) economics. Exemplary diafiltration membranes include Hydrosart 30 kD.

The exchange medium used during solvent removal need not correspond to the medium of the final liposomal adjuvant, for convenience the exchange medium is suitably the desired final liposomal adjuvant medium or a concentrate thereof e.g. phosphate buffered saline or another buffered composition as desired.

In certain methods, the saponin may be added to the recovered mixed material before removal of the solvent. In other methods the saponin may be added after removal of the solvent.

### Saponins

A suitable saponin for use in the present invention is Quil A and its derivatives. Quil A is a saponin preparation isolated from the South American tree Quillaja saponaria Molina and was first described as having adjuvant activity by Dalsgaard et al. in 1974 (“Saponin adjuvants”, Archiv. für die gesamte Virusforschung, Vol. 44, Springer Verlag, Berlin, p 243-254). Purified fractions of Quil A have been isolated by HPLC which retain adjuvant activity without the toxicity associated with Quil A (see, for example, EP0362278). Fractions of general interest include QS7, QS17, QS18 and QS-21, for example QS7 and QS-21 (also known as QA7 and QA21). QS-21 is a saponin fraction of particular interest.

In certain embodiments of the present invention, the saponin is a derivative of Quillaja saponaria Molina quil A, suitably an immunologically active fraction of Quil A, such as QS7, QS17, QS18 or QS-21, in particular QS-21. WO2019106192, incorporated herein by reference for the purpose of defining saponin fractions which may be of use in the present invention, describes QS-21 fractions of particular interest.

Typically the saponin, such as Quil A and in particular QS-21, is at least 90% pure, such as at least 95% pure, especially at least 98% pure, in particular 99% pure w/w.

QS-21 contains a plurality of components, with the principal components typically being:
‘QS-21 1988 A component’, which is identified in Kite 2004 as Peak 88 and corresponds to the A-isomer xylose chemotype structures S4 (apiose isomer) and S6 (xylose isomer) characterised in Nyberg 2000 and Nyberg 2003. The QS-21 1988 A component may consist of QS-21 1988 A V1 (i.e. apiose isomer):

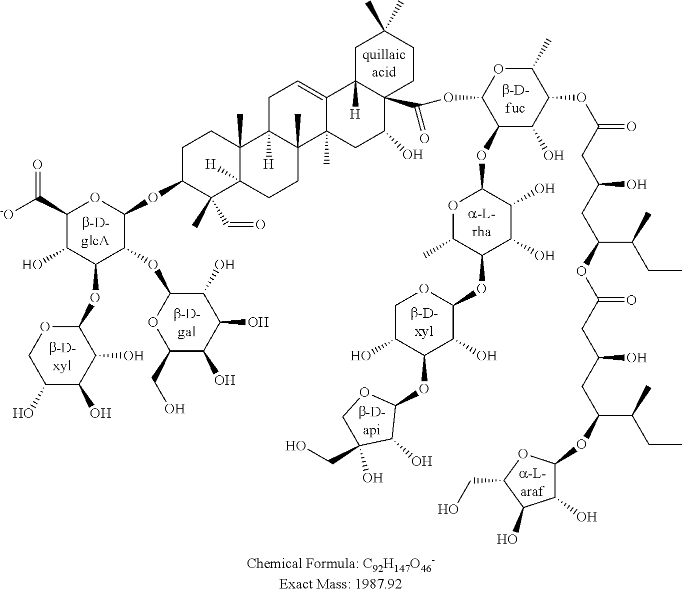

and QS-21 1988 A V2 (i.e. xylose isomer):

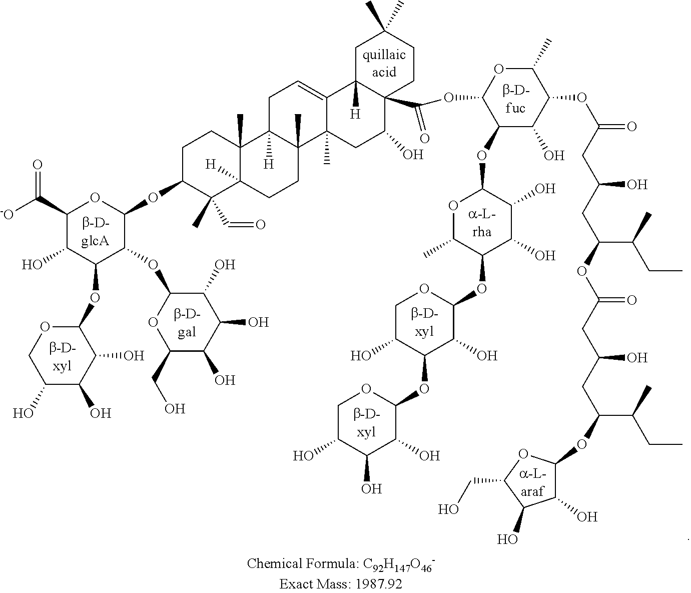

‘QS-21 1856 A component’, which is identified in Kite 2004 as Peak 86 and corresponds to the A-isomer xylose chemotype structure S2 characterised in Nyberg 2000 and Nyberg 2003. The QS-21 1856 A component may consist of:

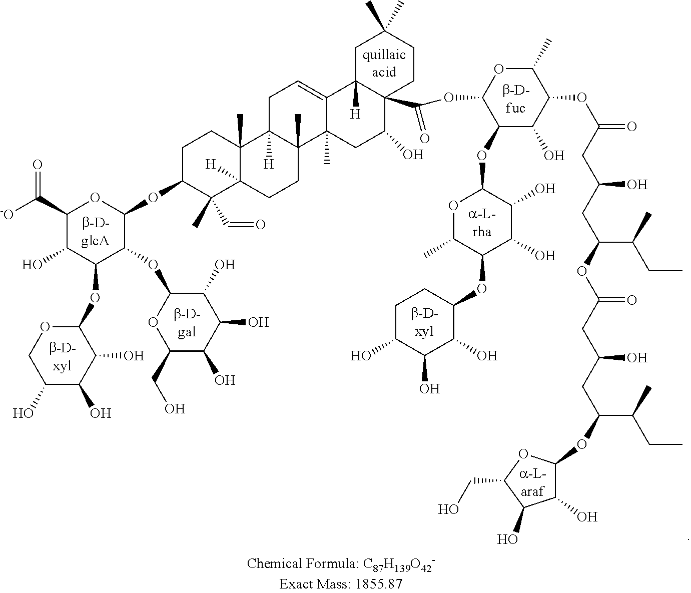

‘QS-21 2002 A component’, which is identified in Kite 2004 as Peak 85 and corresponds to the A-isomer rhamnose chemotype of structures S3 and S5 characterised in Nyberg 2000 and Nyberg 2003. The QS-21 2002 A component may consist of QS-21 2002 A V1 (i.e. apiose isomer):

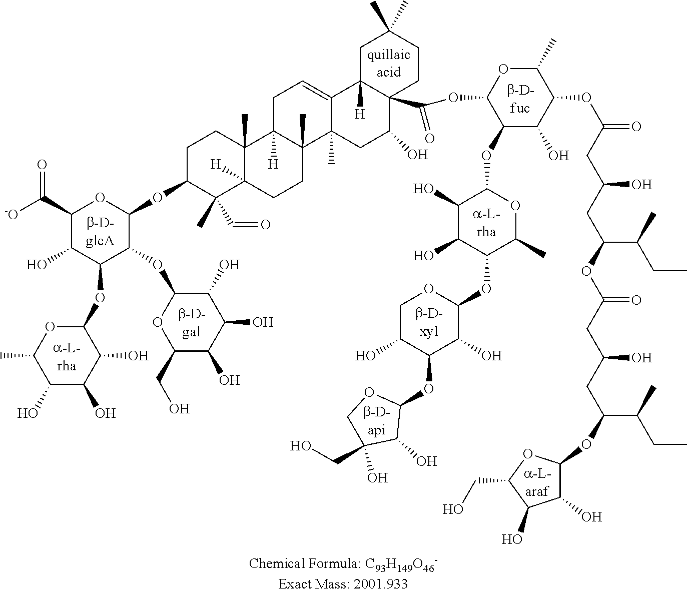

and QS-21 2002 A V2 (i.e. xylose isomer):

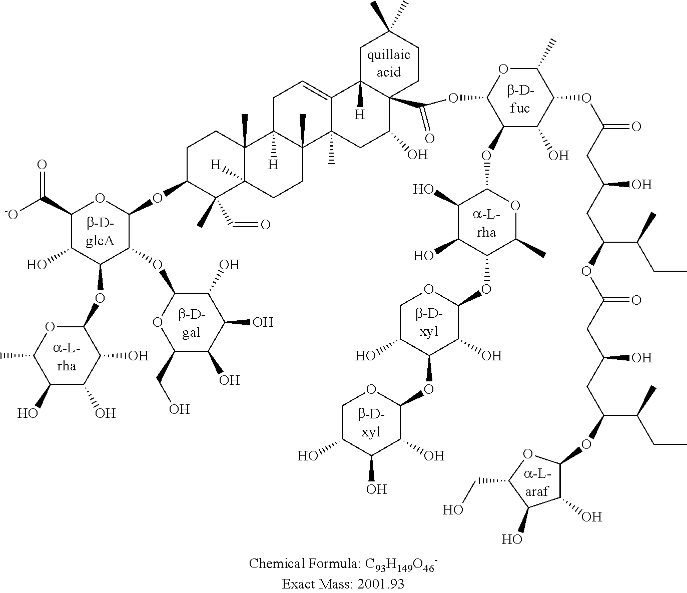

Consequently, the saponin desirably comprises at least 40%, such as at least 50%, suitably at least 60%, especially at least 70% and desirably at least 80%, for example at least 90% (as determined by UV absorbance at 214 nm and by relative ion abundance) QS-21 1988 A component, QS-21 1856 A component and/or QS-21 2002 A component.

In certain embodiments, the saponin contains at least 40%, such as at least 50%, in particular at least 60%, especially at least 65%, such as at least 70%, QS-21 1988 A component as determined by UV absorbance at 214 nm and by relative ion abundance. In certain embodiments the saponin extracts contain 90% or less, such as 85% or less, or 80% or less, QS-21 1988 A component as determined by UV absorbance at 214 nm and by relative ion abundance. In certain embodiments, the saponin extracts contain from 40% to 90% QS-21 1988 A component, such as 50% to 85% QS-21 1988 A component, especially 70% to 80% QS-21 1988 A component as determined by UV absorbance at 214 nm and by relative ion abundance.

In certain embodiments, the saponin extracts contain 30% or less, such as 25% or less, QS-21 1856 A as determined by UV absorbance at 214 nm and by relative ion abundance. In certain embodiments the saponin extracts contain at least 5%, such as at least 10% QS-21 1856 A by UV absorbance at 214 nm and by relative ion abundance. In certain embodiments, the saponin extracts contain from 5% to 30% QS-21 1856 A, such as 10% to 25% QS-21 1856 A as determined by UV absorbance at 214 nm and by relative ion abundance.

In certain embodiments, the saponin extracts contain 40% or less, such as 30% or less, in particular 20% or less, especially 10% or less QS-21 2002 A component by UV absorbance at 214 nm and by relative ion abundance. In certain embodiments, the saponin extracts contain at least 0.5%, such as at least 1%, QS-21 2002 A component by UV absorbance at 214 nm and by relative ion abundance. In certain embodiments, the saponin extracts contain from 0.5% to 40% QS-21 2002 A component, such as 1% to 10% QS-21 2002 A component as determined by UV absorbance at 214 nm and by relative ion abundance.

By the term ‘UV absorbance at 214 nm and relative ion abundance’ is meant an estimate for the percentage of a given m/z for co-eluting species. (Percentage area for given UV peak)×(relative ion abundance for m/z of interest in given peak)/(sum of all relative ion abundance for given peak)=percentage of m/z of interest in the given UV peak, assumes relative ion abundance included for all coeluting species.

QS-21 1988 A component, QS-21 1856 A component and/or QS-21 2002 A component may be obtained by extraction from Quillaja species or may be prepared synthetically (such as semi-synthetically).

A beneficial feature of the present invention is that the saponin is presented in a less reactogenic composition where it is quenched with an exogenous sterol, such as cholesterol.

In methods where the saponin is added after mixing of the first and second solutions, the amount of saponin will typically be equivalent to the amounts which would be used if added earlier.

### TLR4 Agonists

A suitable example of a TLR4 agonist is a lipopolysaccharide, suitably a non-toxic derivative of lipid A, particularly a monophosphoryl lipid A and more particularly 3-de-O-acylated monophosphoryl lipid A (3D-MPL).

3D-MPL is sold under the name ‘MPL’ by GlaxoSmithKline Biologicals N.A. and is referred throughout the document as 3D-MPL. See, for example, U.S. Pat. Nos. 4,436,727; 4,877,611; 4,866,034 and 4,912,094. 3D-MPL can be produced according to the methods described in GB 2 220 211 A. Chemically it is a mixture of 3-deacylated monophosphoryl lipid A with 4, 5 or 6 acylated chains. In the context of the present invention small particle 3D-MPL may be used to prepare the aqueous adjuvant composition. Small particle 3D-MPL has a particle size such that it may be sterile-filtered through a 0.22 um filter. Such preparations are described in WO94/21292. Suitably, powdered 3D-MPL is used to prepare aqueous adjuvant compositions of use in the present invention.

Other TLR4 agonists which can be used are aminoalkyl glucosaminide phosphates (AGPs) such as those described in WO98/50399 or U.S. Pat. No. 6,303,347 (processes for preparation of AGPs are also described). Some AGPs are TLR4 agonists, and some are TLR4 antagonists. A particular AGP of interest is are set forth as follows:

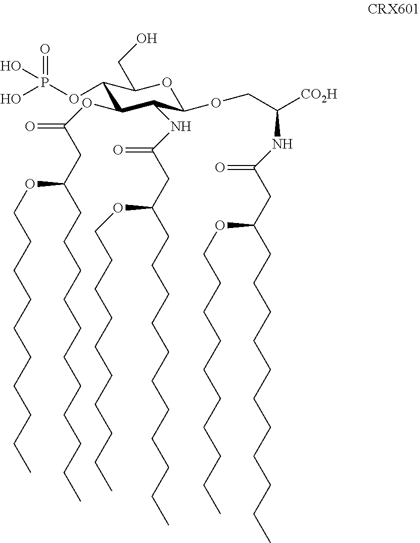

Other TLR4 agonists which may be of use in the present invention include Glucopyranosyl Lipid Adjuvant (GLA) such as described in WO2008153541 or WO2009143457 or the literature articles Coler R N et al. (2011) Development and Characterization of Synthetic Glucopyranosyl Lipid Adjuvant System as a Vaccine Adjuvant. PLoS ONE 6(1): e16333.

doi:10.1371/journal.pone.0016333 and Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144. WO2008153541 or WO2009143457 are incorporated herein by reference for the purpose of defining TLR4 agonists which may be of use in the present invention.

TLR4 agonists of interest include:

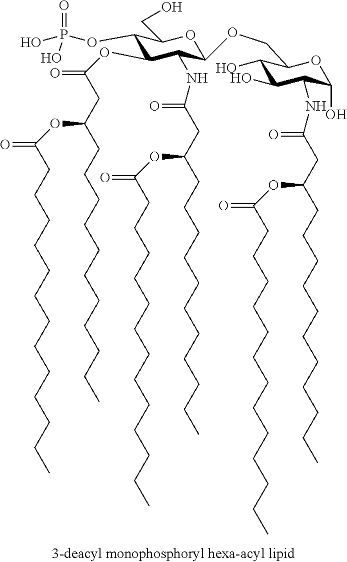

Another TLR4 agonist of interest is:

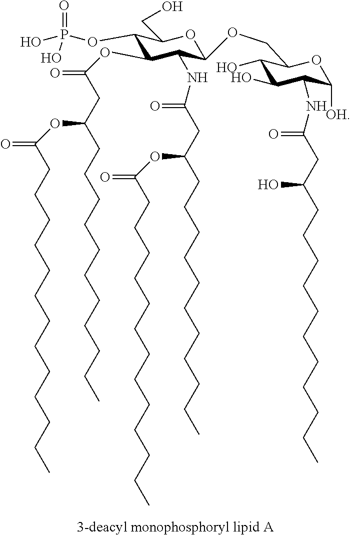

A TLR agonist of interest is dLOS (as described in Han, 2014):

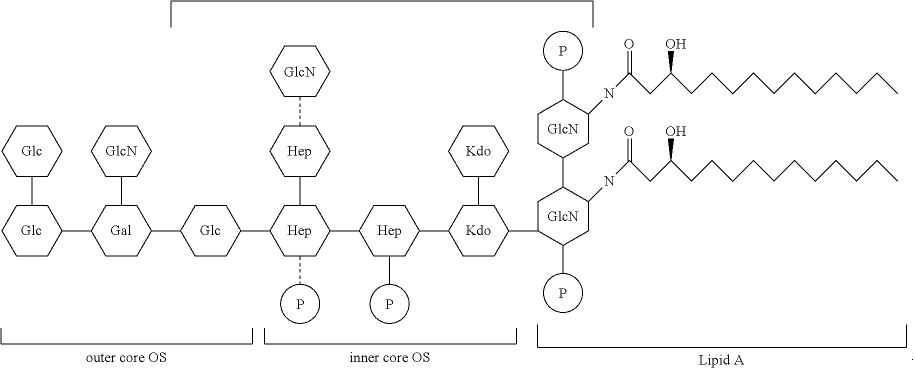

Typically the TLR4 agonist, such as the lipopolysaccharide, such as a monophosphoryl lipid A and in particular 3D-MPL, is at least 90% pure, such as at least 95% pure, especially at least 98% pure, in particular 99% pure w/w.

By the term ‘performed in a single step’ as used herein is intended contemporaneously or simultaneously.

The liposome containing solution obtainable by (such as obtained by) mixing of the first solution and the second solution according to any of the methods described herein forms a further aspect of the invention.

A particular adjuvant of interest features liposomes comprising DOPC and cholesterol, with TLR4 agonist and saponin, especially 3D-MPL and QS-21.

Another adjuvant of interest features liposomes comprising DOTAP and DMPC, with TLR4 agonist and saponin, especially dLOS and QS-21.

### Further Excipients

The liposomal adjuvant resulting from the claimed methods may be further modified. For example, it may be diluted to achieve a particular concentration of components as desired for later uses and/or additional components added. Such steps can be taken at a number of stages in the methods: prior to solvent removal, during solvent removal (e.g. by way of the exchange medium) or after solvent removal.

In a further embodiment, a buffer is added to the composition. The pH of a liquid preparation is adjusted in view of the components of the composition and necessary suitability for administration to the subject. Suitably, the pH of a liquid mixture is at least 4, at least 5, at least 5.5, at least 5.8, at least 6. The pH of the liquid mixture may be less than 9, less than 8, less than 7.5 or less than 7. In other embodiments, pH of the liquid mixture is between 4 and 9, between 5 and 8, such as between 5.5 and 8. Consequently, the pH will suitably be between 6 to 9, such as 6.5 to 8.5. In a particularly preferred embodiment the pH is between 5.8 and 6.4.

An appropriate buffer may be selected from acetate, citrate, histidine, maleate, phosphate, succinate, tartrate and TRIS. In one embodiment, the buffer is a phosphate buffer such as Na/Na₂PO₄, Na/K₂PO₄ or K/K₂PO₄.

The buffer can be present in the liquid mixture in an amount of at least 6 mM, at least 10 mM or at least 40 mM. The buffer can be present in the liquid mixture in an amount of less than 100 mM, less than 60 mM or less than 40 mM.

It is well known that for parenteral administration solutions should have a pharmaceutically acceptable osmolality to avoid excessive cell distortion or lysis. A pharmaceutically acceptable osmolality will generally mean that solutions will have an osmolality which is approximately isotonic or mildly hypertonic. Suitably the compositions of the present invention when reconstituted will have an osmolality in the range of 250 to 750 mOsm/kg, for example, the osmolality may be in the range of 250 to 550 mOsm/kg, such as in the range of 280 to 500 mOsm/kg. In a particularly preferred embodiment the osmolality may be in the range of 280 to 310 mOsm/kg.

Osmolality may be measured according to techniques known in the art, such as by the use of a commercially available osmometer, for example the Advanced™ Model 2020 available from Advanced Instruments Inc. (USA).

An “isotonicity agent” is a compound that is physiologically tolerated and imparts a suitable tonicity to a formulation to prevent the net flow of water across cell membranes that are in contact with the formulation. In some embodiments, the isotonicity agent used for the composition is a salt (or mixtures of salts), conveniently the salt is sodium chloride, suitably at a concentration of approximately 150 nM. In other embodiments, however, the composition comprises a non-ionic isotonicity agent and the concentration of sodium chloride in the composition is less than 100 mM, such as less than 80 mM, e.g. less than 50 mM, such as less 40 mM, less than 30 mM and especially less than 20 mM. The ionic strength in the composition may be less than 100 mM, such as less than 80 mM, e.g. less than 50 mM, such as less 40 mM or less than 30 mM.

In a particular embodiment, the non-ionic isotonicity agent is a polyol, such as sucrose and/or sorbitol. The concentration of sorbitol may e.g. between about 3% and about 15% (w/v), such as between about 4% and about 10% (w/v). Adjuvants comprising an immunologically active saponin fraction and a TLR4 agonist wherein the isotonicity agent is salt or a polyol have been described in WO2012080369.

Suitably, a human dose volume of between 0.05 ml and 1 ml, such as between 0.1 and 0.5 ml, in particular a dose volume of about 0.5 ml, or 0.7 ml. The volumes of the compositions used may depend on the delivery route and location, with smaller doses being given by the intradermal route. A unit dose container may contain an overage to allow for proper manipulation of materials during administration of the unit dose.

The saponin, such as QS-21, can be used at amounts between 1 and 100 ug per human dose. QS-21 may be used at a level of about 50 ug. Examples of suitable ranges are 40 to 60 ug, suitably 45 to 55 ug or 49 to 51 ug, such as 50 ug. In a further embodiment, the human dose comprises QS-21 at a level of about 25 ug. Examples of lower ranges include 20 to 30 ug, suitably 22 to 28 ug or 24 to 26 ug, such as 25 ug. Human doses intended for children may be reduced compared to those intended for an adult (e.g. reduction by 50%).

The TLR4 agonist such as a lipopolysaccharide, such as a monophosphoryl lipid A, such as 3D-MPL, can be used at amounts between 1 and 100 ug per human dose. 3D-MPL may be used at a level of about 50 ug. Examples of suitable ranges are 40 to 60 ug, suitably 45 to 55 ug or 49 to 51 ug, such as 50 ug. In a further embodiment, the human dose comprises 3D-MPL at a level of about ug. Examples of lower ranges include 20 to 30 ug, suitably 22 to 28 ug or 24 to 26 ug, such as 25 ug. Human doses intended for children may be reduced compared to those intended for an adult (e.g. reduction by 50%).

When both a TLR4 agonist and a saponin are present in the adjuvant, then the weight ratio of TLR4 agonist to saponin is suitably between 1:5 to 5:1, suitably 1:1. For example, where 3D-MPL is present at an amount of 50 ug or 25 ug, then suitably QS-21 may also be present at an amount of 50 ug or 25 ug per human dose.

The ratio of saponin:lipid will typically be in the order of 1:50 to 1:10 (w/w), suitably between 1:25 to 1:15 (w/w), and preferably 1:22 to 1:18 (w/w), such as 1:20 (w/w).

The ratio of saponin:DOPC will typically be in the order of 1:50 to 1:10 (w/w), suitably between 1:25 to 1:15 (w/w), and preferably 1:22 to 1:18 (w/w), such as 1:20 (w/w).

The ratio of DOPC:sterol, such as cholesterol, will typically be in the order of 10:1 to 1:1 (w/w), suitably between 8:1 to 2:1 (w/w), and preferably 6:1 to 2.6:1 (w/w), such as about 4:1 (w/w).

Some of the components used may form salts, therefore may be present as a salt, in particular a pharmaceutically acceptable salt.

### Antigens

The liposomal adjuvants prepared according to the methods of the present invention may be utilised in conjunction with an immunogen or antigen. In some embodiments a polynucleotide encoding the immunogen or antigen is provided.

The liposomal adjuvant may be administered separately from an immunogen or antigen may be combined, either during manufacturing or extemporaneously, with an immunogen or antigen as an immunogenic composition for combined administration.

Consequently, there is provided a method for the preparation of an immunogenic composition comprising an immunogen or antigen, or a polynucleotide encoding the immunogen or antigen, said method comprising the steps of:
(i) preparing a liposomal adjuvant according to the methods described herein;
(ii) mixing the liposomal adjuvant with an immunogen or antigen, or a polynucleotide encoding the immunogen or antigen.

There is also provided the use of a liposomal adjuvant prepared according to the methods described herein in the manufacture of a medicament. Suitably the medicament comprises an immunogen or antigen, or a polynucleotide encoding the immunogen or antigen.

Further provided is a liposomal adjuvant prepared according to the methods described herein for use as a medicament. Suitably the medicament comprises an immunogen or antigen, or a polynucleotide encoding the immunogen or antigen.

By the term immunogen is meant a polypeptide which is capable of eliciting an immune response. Suitably the immunogen is an antigen which comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

The antigen may be derived (such as obtained from) from a human or non-human pathogen including, e.g., bacteria, fungi, parasitic microorganisms or multicellular parasites which infect human and non-human vertebrates, or from a cancer cell or tumor cell.

In one embodiment the antigen is a recombinant protein, such as a recombinant prokaryotic protein.

Antigen may be provided in an amount of 0.1 to 100 ug per human dose.

The liposomal adjuvant may be administered separately from an immunogen or antigen, or may be combined, either during manufacturing or extemporaneously), with an immunogen or antigen as an immunogenic composition for combined administration.

### Sterilisation

For parenteral administration in particular, compositions should be sterile. Sterilisation can be performed by various methods although is conveniently undertaken by filtration through a sterile grade filter. Sterilisation may be performed a number of times during preparation of an adjuvant or immunogenic composition, but is typically performed at least at the end of manufacture.

By “sterile grade filter” it is meant a filter that produces a sterile effluent after being challenged by microorganisms at a challenge level of greater than or equal to 1×10⁷/cm² of effective filtration area. Sterile grade filters are well known to the person skilled in the art of the invention for the purpose of the present invention, sterile grade filters have a pore size between 0.15 and 0.25 um, suitably 0.18 to 0.22 um, such as 0.2 or 0.22 um.

The membranes of the sterile grade filter can be made from any suitable material known to the skilled person, for example, but not limited to cellulose acetate, polyethersulfone (PES), polyvinylidene fluoride (PVDF), polytetrafluoroethylene (PTFE). In a particular embodiment of the invention one or more or all of the filter membranes of the present invention comprise polyethersulfone (PES), in particular hydrophilic polyethersulfone. In a particular embodiment of the invention, the filters used in the processes described herein are a double layer filter, in particular a sterile filter with build-in prefilter having larger pore size than the pore size of the end filter. In one embodiment the sterilizing filter is a double layer filter wherein the pre-filter membrane layer has a pore size between 0.3 and 0.5 nm, such as 0.35 or 0.45 nm. According to further embodiments, filters comprise asymmetric filter membrane(s), such as asymmetric hydrophilic PES filter membrane(s). Alternatively, the sterilizing filter layer may be made of PVDF, e.g. in combination with an asymmetric hydrophilic PES pre-filter membrane layer.

In light of the intended medical uses, materials should be of pharmaceutical grade (such as parenteral grade).

By the term ‘substantially’ in respect of an integer is meant functionally comparable, such that deviation may be tolerated if the essential nature of the integer is not changed. Substantially is used herein in respect of terms such as symmetrical/symmetrically, centrally, located at the outer walls, rectangular, the same, parallel, linear, evenly, consistent, inconsistent, opposite, equidistant, different, identical and trapezium.

In respect of numerical values, the term ‘substantially’ or ‘about’ will typically mean a value within plus or minus 10 percent of the stated value, especially within plus or minus 5 percent of the stated value and in particular the stated value.

Substantially parallel will typically mean within 15 degrees, such as within 10 degrees, in particular within 5 degrees, such as parallel.

The term ‘um’ is used herein to mean micrometres.

The teaching of all references in the present application, including patent applications and granted patents, are herein fully incorporated by reference, as if each individual publication were specifically and individually indicated to be incorporated by reference herein as though fully set forth.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. A composition or method or process defined as “comprising” certain elements is understood to encompass a composition, method or process (respectively) consisting of those elements. As used herein, ‘consisting essentially of’ means additional components may be present provided they do not alter the overall properties or function.

The invention is further illustrated by reference to the following clauses:
Clause 1. A microfluidic mixing device comprising: a mixing chamber; one inlet channel into the mixing chamber for a first fluid and two inlet channels into the mixing chamber for a second fluid, said inlet channels being disposed substantially symmetrically at a proximal end of the mixing chamber; at least one outlet for mixed material at a distal end of the mixing chamber; characterised in that the mixing chamber comprises one or more baffles.
Clause 2. The device according to clause 1, wherein the inlet channel for the first fluid is substantially rectangular in cross-section.
Clause 3. The device according to clause 2, wherein the inlet channel for the first fluid is 0.1 to 0.7 mm wide, such as 0.15 to 0.5 mm wide, especially 0.15 to 0.5 mm wide and in particular 0.2 to 0.35 mm wide.
Clause 4. The device according to clause 3, wherein the inlet channel for the first fluid is 0.25 to 0.3 mm wide, such as 0.27 mm wide.
Clause 5. The device according to any one of clauses 2 to 4, wherein the inlet channel for the first fluid is at least 0.05 mm deep, such as at least 0.1 mm deep, especially at least 0.2 mm deep and in particular at least 0.3 mm deep.
Clause 6. The device according to clause 5, wherein the inlet channel for the first fluid is at least 0.4 mm deep.
Clause 7. The device according to any one of clauses 2 to 6, wherein the inlet channel for the first fluid is 10 mm deep or less, such as 5 mm deep or less, especially 2 mm deep or less and in particular 1 mm deep or less.
Clause 8. The device according to clause 7, wherein the inlet channel for the first fluid is 0.8 mm deep or less.
Clause 9. The device according to any one of clause 2 to 8, wherein the inlet channel for the first fluid is 0.1 to 2 mm deep, such as 0.3 to 0.8 mm deep, especially 0.4 to 0.6 mm deep and in particular about 0.5 mm deep such as 0.5 mm deep.
Clause 10. The device according to any one of clause 1 to 9, wherein the inlet channel for the first fluid has a cross-sectional area of 0.01 to 4 mm², such as 0.05 to 1 mm² and especially 0.08 to 0.4 mm².
Clause 11. The device according to clause 10, wherein the inlet channel for the first fluid has a cross-sectional area of 0.1 to 0.2 mm², in particular about 0.135 mm², such as 0.135 mm².
Clause 12. The device according to any one of clauses 1 to 11, wherein the depth of the inlet channel for the first fluid is substantially the same as the depth of the mixing chamber.
Clause 13. The device according to any one of clauses 1 to 12, wherein the direction of flow from the inlet channel for the first fluid into the mixing chamber is substantially parallel to the general direction of flow in the mixing chamber.
Clause 14. The device according to any one of clauses 1 to 13, wherein the inlet channel for the first fluid is substantially linear for at least 3 mm, such as at least 5 mm, especially at least 7 mm.
Clause 15. The device according to any one of clauses 1 to 14, wherein the inlet channels for the second fluid are substantially identical in shape.
Clause 16. The device according to any one of clauses 1 to 15, wherein the inlet channels for the second fluid are substantially rectangular in cross-section.
Clause 17. The device according to clause 16, wherein the inlet channels for the second fluid are 0.025 to 0.3 mm wide, such as 0.05 to 0.25 mm wide.
Clause 18. The device according to clause 17, wherein the inlet channels for the second fluid are 0.08 to 0.12 mm wide, such as 0.1 mm wide.
Clause 19. The device according to any one of clauses 15 to 18, wherein the inlet channels for the second fluid are at least 0.05 mm deep, such as at least 0.1 mm deep, especially at least 0.2 mm deep and in particular at least 0.3 mm deep.
Clause 20. The device according to clause 19, wherein the inlet channels for the second fluid are at least 0.4 mm deep.
Clause 21. The device according to any one of clauses 15 to 20, wherein the inlet channels for the second fluid are 10 mm deep or less, such as 5 mm deep or less, especially 2 mm deep or less and in particular 1 mm deep or less.
Clause 22. The device according to clause 21, wherein the inlet channels for the second fluid are 0.8 mm deep or less.
Clause 23. The device according to any one of clauses 15 to 22, wherein the inlet channels for the second fluid are 0.1 to 2 mm deep, such as 0.3 to 0.8 mm deep, especially 0.4 to 0.6 mm deep and in particular about 0.5 mm deep such as 0.5 mm deep.
Clause 24. The device according to any one of clauses 1 to 23, wherein the inlet channels for the second fluid have a cross-sectional area of 0.005 to 3 mm², such as 0.01 to 0.5 mm².
Clause 25. The device according to clause 24, wherein the inlet channels for the second fluid have a cross-sectional area of 0.02 to 0.1 mm², in particular about 0.05 mm², such as 0.05 mm².
Clause 26. The device according to any one of clauses 1 to 25, wherein the inlet channels for the second fluid are substantially the same as the depth of the mixing chamber.
Clause 27. The device according to any one of clauses 1 to 26, wherein the direction of flow from the inlet channels for the second fluid into the mixing chamber is substantially parallel to the general direction of flow in the mixing chamber.
Clause 28. The device according to any one of clauses 1 to 27, wherein the inlet channels for the second fluid are substantially linear for at least 3 mm, such as at least 5 mm, especially at least 7 mm and in particular at least 10 mm.
Clause 29. The device according to any one of clauses 1 to 28, wherein the direction of flow from the inlet channels for the second fluid into the mixing chamber is substantially parallel to the direction of flow from the inlet channel for the first fluid in the mixing chamber.
Clause 30. The device according to any one of clauses 1 to 29, wherein the length of the mixing chamber is at least 15 mm, such as at least 17.5 mm, especially at least 20 mm, in particular at least 22 mm.
Clause 31. The device according to any one of clauses 1 to 30, wherein the length of the mixing chamber is 100 mm or less, such as 75 mm or less, especially 50 mm or less, in particular 40 mm or less.
Clause 32. The device according to any one of clauses 1 to 31, wherein the length of the mixing chamber is 15 to 100 mm, such as 17.5 to 75 mm, especially 20 mm to 50 mm, in particular about 25 mm, such as 25 mm.
Clause 33. The device according to any one of clauses 1 to 32, wherein the mixing chamber is substantially rectangular in cross-section.
Clause 34. The device according to clause 33, wherein the larger dimension may be one and a half to four times that of the perpendicular dimension.
Clause 35. The device according to any one of clauses 1 to 34, wherein the maximum width of the mixing chamber is 0.8 to 2.2 mm, such as 1 to 2 mm, especially 1.2 to 2 mm.
Clause 36. The device according to clause 35, wherein the maximum width of the mixing chamber is 1.4 to 1.8 mm, in particular about 1.6 mm, such as 1.6 mm.
Clause 37. The device according to clause 35, wherein the maximum width of the mixing chamber is 0.8 to 1.2 mm, in particular about 1 mm, such as 1 mm.
Clause 38. The device according to any one of clauses 1 to 37, wherein the minimum width of the mixing chamber is 0.8 to 2.2 mm, such as 1 to 2 mm, especially 1.2 to 2 mm.
Clause 39. The device according to clause 38, wherein the minimum width of the mixing chamber is 1.4 to 1.8 mm, in particular about 1.6 mm, such as 1.6 mm.
Clause 40. The device according to clause 38, wherein the minimum width of the mixing chamber is 0.8 to 1.2 mm, in particular about 1 mm, such as 1 mm.
Clause 41. The device according to any one of clauses 1 to 38, wherein the maximum width of the mixing chamber and minimum width of the mixing chamber are the substantially the same, such as the same.
Clause 42. The device according to any one of clauses 1 to 37, wherein the minimum width of the mixing chamber is 0.4 to 1.2 mm, such as 0.6 to 0.9 mm, especially about 0.75 mm, such as 0.75 mm.
Clause 43. The device according to any one of clauses 1 to 42, wherein the mixing chamber is at least 0.05 mm deep, such as at least 0.1 mm deep, especially at least 0.2 mm deep and in particular at least 0.3 mm deep.
Clause 44. The device according to clause 43, wherein the mixing chamber is at least 0.4 mm deep.
Clause 45. The device according to any one of clauses 1 to 44, wherein the mixing chamber is 10 mm deep or less, such as 5 mm deep or less, especially 2 mm deep or less and in particular 1 mm deep or less.
Clause 46. The device according to clause 45, wherein the mixing chamber is 0.8 mm deep or less.
Clause 47. The device according to any one of clauses 1 to 46, wherein the mixing chamber is 0.1 to 2 mm deep, such as 0.3 to 0.8 mm deep, especially 0.4 to 0.6 mm deep and in particular about 0.5 mm deep such as 0.5 mm deep.
Clause 48. The device according to any one of clauses 1 to 47, wherein the mixing chamber is of substantially consistent depth along its length, such as of consistent depth along its length.
Clause 49. The device according to any one of clauses 1 to 47, wherein the mixing chamber is of reducing depth along its length, such as reduced depth by up to 50%.
Clause 50. The device according to any one of clauses 1 to 49, wherein the mixing chamber width is 1 to 5 times the mixing chamber depth.
Clause 51. The device according to any one of clauses 1 to 50, wherein the mixing chamber has a cross-sectional area of 0.1 to 2.2 mm², such as 0.2 to 1.8 mm², especially 0.4 to 1.6 mm², in particular 0.6 to 1.0 mm², such as about 0.8 mm², such as 0.8 mm².
Clause 52. The device according to any one of clauses 1 to 50, wherein the mixing chamber has a cross-sectional area of 0.2 to 0.8 mm², such as 0.3 to 0.7 mm², especially 0.4 to 0.6 mm², such as about 0.5 mm², such as 0.5 mm².
Clause 53. The device according to any one of clauses 1 to 50, wherein the mixing chamber has a cross-sectional area of 0.4 to 1.0 mm².
Clause 54. The device according to any one of clauses 1 to 50, wherein the mixing chamber has a cross-sectional area of 0.25 to 0.6 mm².
Clause 55. The device according to any one of clauses 1 to 54, wherein baffles are present on one side of the mixing chamber.
Clause 56. The device according to any one of clauses 1 to 55, wherein baffles are present on both sides of the mixing chamber.
Clause 57. The device according to any one of clauses 1 to 56, wherein baffles on at least one side of the mixing chamber are spaced substantially evenly.
Clause 58. The device according to clause 57, wherein baffles on both sides of the mixing chamber are spaced substantially evenly.
Clause 59. The device according to any one of clauses 1 to 58, wherein baffles on at least one side of the mixing chamber are spaced substantially unevenly.
Clause 60. The device according to clause 59, wherein baffles on both sides of the mixing chamber are spaced substantially unevenly.
Clause 61. The device according to any one of clauses 1 to 60, comprising baffles on both sides of the mixing chamber which are positioned substantially opposite each other.
Clause 62. The device according to any one of clauses 1 to 61, comprising baffles on both sides of the mixing chamber which are positioned offset to each other, such as by 0.5 to 5 mm, such as 1 to 2.5 mm and in particular about 1.75 mm such as 1.732 mm.
Clause 63. The device according to any one of clauses 1 to 62, comprising baffles on both sides of the mixing chamber which are positioned offset to each other by about 1.26 mm such as 1.258 mm.
Clause 64. The device according to any one of clauses 1 to 62, comprising baffles on both sides of the mixing chamber which are positioned offset to each other by about 1 mm, such as 1 mm.
Clause 65. The device according to any one of clauses 1 to 62, comprising baffles on both sides of the mixing chamber which are positioned offset to each other by about 0.728 mm, such as 0.728 mm.
Clause 66. The device according to any one of clauses 63 to 65, wherein the offset before and after are substantially the same.
Clause 67. The device according to any one of clauses 63 to 65, wherein the offset before and after are substantially different.
Clause 68. The device according to any one of clauses 1 to 67, comprising at least 4 baffles, such as at least 6 baffles, especially at least 8 baffles, in particular at least 10 baffles.
Clause 69. The device according to any one of clauses 1 to 68, comprising 100 or fewer baffles, such as 60 or fewer baffles, especially 40 or fewer baffles, in particular 25 or fewer baffles.
Clause 70. The device according to any one of clauses 1 to 69, comprising 4 to 100 baffles, such as 6 to 60 baffles, especially 8 to 40 baffles, in particular 10 to 25 baffles.
Clause 71. The device according to clause 70, comprising 12 baffles.
Clause 72. The device according to clause 70, comprising 19 baffles.
Clause 73. The device according to any one of clauses 1 to 72, comprising baffles of wave, bell or trapezium shape.
Clause 74. The device according to any one of clauses 1 to 73, wherein all baffles have substantially the same shape.
Clause 75. The device according to any one of clauses 1 to 74, comprising baffles of constant width.
Clause 76. The device according to any one of clauses 1 to 74, comprising baffles of variable width.
Clause 77. The device according to any one of clauses 1 to 76, comprising baffles 0.1 to 1 mm wide, such as 0.2 to 0.8 mm wide, especially 0.4 to 0.7 mm wide, in particular about 0.5 mm wide such as 0.5 mm wide.
Clause 78. The device according to any one of clauses 1 to 76, comprising baffles 0.2 to 0.5 mm wide, such as 0.3 to 0.4 mm wide, in particular about 0.35 mm wide such as 0.35 mm wide.
Clause 79. The device according to any one of clauses 1 to 78, wherein the mixing chamber width at a baffle is reduced by at least 10%, such as at least 20%, especially at least 25% and in particular at least 30%.
Clause 80. The device according to any one of clauses 1 to 79, wherein the mixing chamber width at a baffle is reduced by 80% or less, such as 60% or less, especially 50% or less and in particular 40% or less.
Clause 81. The device according to any one of clauses 1 to 80, wherein the mixing chamber width at a baffle is reduced by 10 to 80%, such as 20 to 60%, especially 35 to 50% and in particular to 40%.
Clause 82. The device according to any one of clauses 1 to 78, wherein the mixing chamber width at a baffle is reduced by 30 to 50%.
Clause 83. The device according to any one of clauses 1 to 82, wherein the mixing chamber width at a baffle is 0.5 to 2 mm, such as 0.7 to 1.5 mm, especially 0.9 to 1.3 mm and in particular about 1.1 mm such as 1.1 mm.
Clause 84. The device according to any one of clauses 1 to 82, wherein the mixing chamber width at a baffle is 0.4 to 0.9 mm, such as 0.5 to 0.8 mm and in particular about 0.65 mm such as 0.65 mm.
Clause 85. The device according to any one of clauses 1 to 84, wherein the mixing chamber comprises baffles on one side which are separated by 1 to 10 mm, such as 2 to 5 mm and in particular about 3.5 mm such as 3.464 mm.
Clause 86. The device according to clause 85, wherein each baffle on one side of the mixing chamber is separated by 1 to 10 mm, such as 2 to 5 mm and in particular about 3.5 mm such as 3.464 mm.
Clause 87. The device according to any one of clauses 1 to 84, wherein the mixing chamber comprises baffles on two sides which are separated by 1 to 10 mm, such as 2 to 5 mm and in particular about 3.5 mm such as 3.464 mm.
Clause 88. The device according to clause 87, wherein each baffle on each side of the mixing chamber is separated by 1 to 10 mm, such as 2 to 5 mm and in particular about 3.5 mm such as 3.464 mm.
Clause 89. The device according to any one of clauses 1 to 84, wherein mixing chamber comprises baffles on two sides which are separated by about 2.5 mm such as 2.516 mm
Clause 90. The device according to clause 89, wherein mixing chamber comprises baffles on two sides and each baffle on each side of the mixing chamber is separated by about 2.5 mm such as 2.516 mm.
Clause 91. The device according to any one of clauses 1 to 84, wherein mixing chamber comprises baffles on two sides which are separated by about 2 mm such as 2 mm
Clause 92. The device according to clause 91, wherein mixing chamber comprises baffles on two sides and each baffle on each side of the mixing chamber is separated by about 2 mm such as 2 mm.
Clause 93. The device according to any one of clauses 1 to 92, wherein the first baffle is located 0.2 to 20 mm from the end of the inlet channels for the second fluid, such as 0.4 to 10 mm, especially 0.6 to 8 mm, in particular about 0.8 mm, about 4.4 mm or about 5.3 mm, such as 0.8, 4.4 or 5.3 mm.
Clause 94. The device according to any one of clauses 1 to 93, comprising baffles having a maximum length of 0.1 to 5 mm, such as 0.2 to 2 mm, especially 0.2 to 1 mm and in particular 0.25 to 0.7 mm, such as about 0.33 mm or about 0.55 mm, such as 0.33 mm or 0.55 mm.
Clause 95. The device according to any one of clauses 1 to 94, comprising baffles having a minimum length of 3 mm or less, such as 1 mm or less, especially 0.5 mm or less and in particular 0.3 mm or less, such as about 0.15 mm or about 0.25 mm, such as 0.15 mm or 0.25 mm.
Clause 96. The device according to any one of clauses 1 to 95, wherein the maximum width of the mixing chamber at a baffle is 0.4 to 2 mm, such as 0.5 to 1.6 mm, especially 0.6 to 1.4 mm, in particular about 0.65 mm or about 1.1 mm, such as 0.65 mm or 1.1 mm.
Clause 97. The device according to any one of clauses 1 to 96, wherein the minimum width of the mixing chamber at a baffle is 0.4 to 2 mm, such as 0.5 to 1.6 mm, especially 0.6 to 1.4 mm, in particular about 0.65 mm or about 1.1 mm, such as 0.65 mm or 1.1 mm.
Clause 98. The device according to any one of clauses 1 to 97, wherein the maximum width of the mixing chamber and minimum width of the mixing chamber at a baffle are the same.
Clause 99. The device according to any one of clauses 1 to 98, wherein the sides of the mixing chamber are substantially parallel.
Clause 100. The device according to any one of clauses 1 to 99, wherein the outer walls of the outer inlets are substantially continuous with the sides of the mixing chamber.
Clause 101. The device according to any one of clauses 1 to 100, having a single outlet for the collection of mixed material.
Clause 102. The device according to any one of clauses 1 to 100, having a plurality of outlets, such as two outlets, for the collection of mixed material.
Clause 103. The device according to any one of clauses 1 to 102, which is less than 100 mm in length, such as less than 80 mm, especially less than 60 mm and particularly less than 45 mm.
Clause 104. The device according to any one of clauses 1 to 103, which is less than 20 mm in width, such as less than 10 mm, especially less than 7 mm and particularly less than 5 mm.
Clause 105. A microfluidic mixing device according to any of the preceding clauses comprising:
   a mixing chamber about 25 mm in length, having a substantially rectangular cross-section, substantially parallel sides spaced about 1.6 mm apart and substantially parallel top and bottom walls spaced to provide a depth of about 0.5 mm;
   one inlet channel into the mixing chamber for a first fluid, being substantially centrally located at a proximal end of the mixing chamber, having a substantially rectangular cross-section, a width of about 0.27 mm and a depth of about 0.5 mm;
   two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises about 19 baffles, the first baffle being located about 0.8 mm from the proximal end of the mixing chamber, baffles being separated by about 2.6 mm with an offset between the first and second sides of about 0.728 mm, the baffles being substantially trapezium in shape with a maximum length of about 0.55 mm, a minimum length of about 0.25 mm and a width of about 0.5 mm.
Clause 106. A microfluidic mixing device according to any of the preceding clauses comprising:
   a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
   one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
   two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises 17 to 21 baffles, the first baffle being located 0.72 to 0.88 mm from the proximal end of the mixing chamber, baffles being separated by 2.26 to 2.77 mm with an offset between the first and second sides of 0.656 to 0.8 mm, the baffles being substantially trapezium in shape with a maximum length of 0.495 to 0.605 mm, a minimum length of 0.225 to 0.275 mm and a width of 0.45 to 0.55 mm.
Clause 107. A microfluidic mixing device according to either clause 105 or 106 comprising:
   a mixing chamber 25 mm in length, having a rectangular cross-section, parallel sides spaced 1.6 mm apart and parallel top and bottom walls spaced to provide a depth of 0.5 mm;
   one inlet channel into the mixing chamber for a first fluid, being centrally located at a proximal end of the mixing chamber, having a rectangular cross-section, a width of 0.27 mm and a depth of 0.5 mm;
   two inlet channels into the mixing chamber for a second fluid, being identical, located at each of the outer walls at the proximal end of the mixing chamber, having a rectangular cross-section, a width of 0.1 mm and a depth of 0.5 mm;
   said inlet channels being disposed symmetrically at the proximal end of the mixing chamber and wherein the direction of flow from the inlet channels into the mixing chamber is parallel to the general direction of flow in the mixing chamber;
   one outlet for mixed material at a distal end of the mixing chamber;
characterised in that the mixing chamber comprises 19 baffles, the first baffle being located 0.8 mm from the proximal end of the mixing chamber, baffles being separated by 2.516 mm with an offset between the first and second sides of 0.728 mm, the baffles being trapezium in shape with a maximum length of 0.55 mm, a minimum length of 0.25 mm and a width of 0.5 mm.
Clause 108. A microfluidic mixing device according to any of the preceding clauses comprising:
   a mixing chamber about 25 mm in length, having a substantially rectangular cross-section, substantially parallel sides spaced about 1.6 mm apart and substantially parallel top and bottom walls spaced to provide a depth of about 0.5 mm;
   one inlet channel into the mixing chamber for a first fluid, being substantially centrally located at a proximal end of the mixing chamber, having a substantially rectangular cross-section, a width of about 0.27 mm and a depth of about 0.5 mm;
   two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises about 19 baffles, the first baffle being located about 0.8 mm from the proximal end of the mixing chamber, baffles being separated by about 2.6 mm with an offset between the first and second sides of about 1.3 mm, the baffles being substantially trapezium in shape with a maximum length of about 0.55 mm, a minimum length of about 0.25 mm and a width of about 0.5 mm.
Clause 109. A microfluidic mixing device according to any of the preceding clauses comprising:
   a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
   one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
   two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises 17 to 21 baffles, the first baffle being located 0.72 to 0.88 mm from the proximal end of the mixing chamber, baffles being separated by 2.26 to 2.77 mm with an offset between the first and second sides of 1.13 to 1.38 mm, the baffles being substantially trapezium in shape with a maximum length of 0.495 to 0.605 mm, a minimum length of 0.225 to 0.275 mm and a width of 0.45 to 0.55 mm.
Clause 110. A microfluidic mixing device according to either clause 108 or 109 comprising:
   a mixing chamber 25 mm in length, having a rectangular cross-section, parallel sides spaced 1.6 mm apart and parallel top and bottom walls spaced to provide a depth of 0.5 mm;
   one inlet channel into the mixing chamber for a first fluid, being centrally located at a proximal end of the mixing chamber, having a rectangular cross-section, a width of 0.27 mm and a depth of 0.5 mm;
   two inlet channels into the mixing chamber for a second fluid, being identical, located at each of the outer walls at the proximal end of the mixing chamber, having a rectangular cross-section, a width of 0.1 mm and a depth of 0.5 mm;
   said inlet channels being disposed symmetrically at the proximal end of the mixing chamber and wherein the direction of flow from the inlet channels into the mixing chamber is parallel to the general direction of flow in the mixing chamber;
   one outlet for mixed material at a distal end of the mixing chamber;
characterised in that the mixing chamber comprises 19 baffles, the first baffle being located 0.8 mm from the proximal end of the mixing chamber, baffles being separated by 2.516 mm with an offset between the first and second sides of 1.258 mm, the baffles being trapezium in shape with a maximum length of 0.55 mm, a minimum length of 0.25 mm and a width of 0.5 mm.
Clause 111. A microfluidic mixing device according to any of the preceding clauses comprising:
   a mixing chamber about 25 mm in length, having a substantially rectangular cross-section, substantially parallel sides spaced about 1.6 mm apart and substantially parallel top and bottom walls spaced to provide a depth of about 0.5 mm;
   one inlet channel into the mixing chamber for a first fluid, being substantially centrally located at a proximal end of the mixing chamber, having a substantially rectangular cross-section, a width of about 0.27 mm and a depth of about 0.5 mm;
   two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises about 12 baffles, the first baffle being located about 4.4 mm from the proximal end of the mixing chamber, baffles being separated by about 3.5 mm with an offset between the first and second sides of about 1.7 mm, the baffles being substantially trapezium in shape with a maximum length of about 0.55 mm, a minimum length of about 0.25 mm and a width of about 0.5 mm.
Clause 112. A microfluidic mixing device according to any of the preceding clauses comprising:
   a mixing chamber 22.5 to 27.5 mm in length, having a substantially rectangular cross-section, substantially parallel sides spaced 1.44 to 1.76 mm apart and substantially parallel top and bottom walls spaced to provide a depth of 0.45 to 0.55 mm;
   one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
   two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises 10 to 14 baffles, the first baffle being located 3.96 to 4.84 mm from the proximal end of the mixing chamber, baffles being separated by 3.12 to 3.81 mm with an offset between the first and second sides of 1.56 to 1.91 mm, the baffles being substantially trapezium in shape with a maximum length of 0.495 to 0.605 mm, a minimum length of 0.225 to 0.275 mm and a width of 0.45 to 0.55 mm.
Clause 113. A microfluidic mixing device according to either clause 111 or 112 comprising:
   a mixing chamber 25 mm in length, having a rectangular cross-section, parallel sides spaced 1.6 mm apart and parallel top and bottom walls spaced to provide a depth of 0.5 mm;
   one inlet channel into the mixing chamber for a first fluid, being centrally located at a proximal end of the mixing chamber, having a rectangular cross-section, a width of 0.27 mm and a depth of 0.5 mm;
   two inlet channels into the mixing chamber for a second fluid, being identical, located at each of the outer walls at the proximal end of the mixing chamber, having a rectangular cross-section, a width of 0.1 mm and a depth of 0.5 mm;
   said inlet channels being disposed symmetrically at the proximal end of the mixing chamber and wherein the direction of flow from the inlet channels into the mixing chamber is parallel to the general direction of flow in the mixing chamber;
   one outlet for mixed material at a distal end of the mixing chamber;
characterised in that the mixing chamber comprises 12 baffles, the first baffle being located 4.4 mm from the proximal end of the mixing chamber, baffles being separated by 3.464 mm with an offset between the first and second sides of 1.732 mm, the baffles being trapezium in shape with a maximum length of 0.55 mm, a minimum length of 0.25 mm and a width of 0.5 mm.
Clause 114. A microfluidic mixing device according to any of the preceding clauses comprising:
   a mixing chamber about 25 mm in length, having a substantially rectangular cross-section, substantially parallel sides spaced about 1 mm apart and substantially parallel top and bottom walls spaced to provide a depth of about 0.5 mm;
   one inlet channel into the mixing chamber for a first fluid, being substantially centrally located at a proximal end of the mixing chamber, having a substantially rectangular cross-section, a width of about 0.27 mm and a depth of about 0.5 mm;
   two inlet channels into the mixing chamber for a second fluid, being substantially identical, substantially located at each of the outer walls at the proximal end of the mixing chamber, having a substantially rectangular cross-section, a width of about 0.1 mm and a depth of about 0.5 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises about 19 baffles, the first baffle being located about 5.3 mm from the proximal end of the mixing chamber, baffles being separated by about 2 mm with an offset between the first and second sides of about 1 mm, the baffles being substantially trapezium in shape with a maximum length of about 0.33 mm, a minimum length of about 0.15 mm and a width of about 0.35 mm.
Clause 115. A microfluidic mixing device according to any of the preceding clauses comprising:
   a mixing chamber 22.5 to 27.5 mm in length, having a substantially rectangular cross-section, substantially parallel sides spaced 0.9 to 1.1 mm apart and substantially parallel top and bottom walls spaced to provide a depth of 0.45 to 0.55 mm;
   one inlet channel into the mixing chamber for a first fluid, being substantially centrally located at a proximal end of the mixing chamber, having a substantially rectangular cross-section, a width of 0.243 to 0.297 mm and a depth of 0.45 to 0.55 mm;
   two inlet channels into the mixing chamber for a second fluid, being substantially identical, substantially located at each of the outer walls at the proximal end of the mixing chamber, having a substantially rectangular cross-section, a width of 0.09 to 0.11 mm and a depth of 0.45 to 0.55 mm;
   said inlet channels being disposed substantially symmetrically at the proximal end of the mixing chamber and wherein the direction of flow from the inlet channels into the mixing chamber is substantially parallel to the general direction of flow in the mixing chamber;
   one outlet for mixed material at a distal end of the mixing chamber;
characterised in that the mixing chamber comprises 17 to 21 baffles, the first baffle being located 4.77 to 5.83 mm from the proximal end of the mixing chamber, baffles being separated by 1.8 to 2.2 mm with an offset between the first and second sides of 0.9 to 1.1 mm, the baffles being substantially trapezium in shape with a maximum length of 0.297 to 0.363 mm, a minimum length of 0.135 to 0.165 mm and a width of 0.315 to 0.385 mm.
Clause 116. A microfluidic mixing device according to either clause 114 or 115 comprising:
   a mixing chamber 25 mm in length, having a rectangular cross-section, parallel sides spaced 1 mm apart and parallel top and bottom walls spaced to provide a depth of 0.5 mm;
   one inlet channel into the mixing chamber for a first fluid, being centrally located at a proximal end of the mixing chamber, having a rectangular cross-section, a width of 0.27 mm and a depth of 0.5 mm;
   two inlet channels into the mixing chamber for a second fluid, being identical, located at each of the outer walls at the proximal end of the mixing chamber, having a rectangular cross-section, a width of 0.1 mm and a depth of 0.5 mm;
   said inlet channels being disposed symmetrically at the proximal end of the mixing chamber and wherein the direction of flow from the inlet channels into the mixing chamber is parallel to the general direction of flow in the mixing chamber;
   one outlet for mixed material at a distal end of the mixing chamber;
characterised in that the mixing chamber comprises 19 baffles, the first baffle being located 5.3 mm from the proximal end of the mixing chamber, baffles being separated by 2 mm with an offset between the first and second sides of 1 mm, the baffles being trapezium in shape with a maximum length of 0.33 mm, a minimum length of 0.15 mm and a width of 0.35 mm.
Clause 117. A chip comprising a plurality of microfluidic devices according to any one of clauses 1 to 116, such as 2 to 128, especially 4 to 32, in particular 6 to 24, such as about 8 or about 16, such as 8 or 16.
Clause 118. The chip according to clause 117, comprising 6 to 18 microfluidic devices according to any one of clauses 1 to 116.
Clause 119. The chip according to either clause 117 or 118, having a single point of connection for the first and the second fluid and a single point of connection for collection of the mixed material.
Clause 120. An apparatus comprising a plurality of microfluidic mixing devices according to any one of clauses 1 of 116 or a chip according to any one of clauses 117 to 119, a first pump for the supply of the first fluid and a second pump for the supply of the second fluid, the microfluidic mixing devices being configured for parallel operation using the first and second pumps.
Clause 121. The apparatus according to clause 120, comprising 4 to 20 microfluidic mixing devices according to any one of clauses 1 of 116.
Clause 122. A method of manufacturing a liposomal adjuvant using a microfluidic device according to any one of clauses 1 to 116, a chip according to any one of clauses 117 to 119 or apparatus according to either clause 120 or 121, comprising the following steps:
(a) mixing in the device a first solution comprising a solvent and lipid, and a second solution comprising water; and
(b) removing the solvent.
Clause 123. The method of manufacturing a liposomal adjuvant according to clause 122, comprising the following steps:
(a) mixing in the device a first solution comprising a solvent and lipid, and a second solution comprising water;
(b) adding a saponin; and
(c) removing the solvent.
Clause 124. The method of manufacturing a liposomal adjuvant according to clause 122, comprising the following steps:
(a) mixing in the device a first solution comprising a solvent and lipid, and a second solution comprising water;
(b) removing the solvent; and
(c) adding a saponin.
Clause 125. The method of manufacturing a liposomal adjuvant according to clause 122, comprising the following steps:
(a) mixing in the device a first solution comprising a solvent and lipid, and a second solution comprising water;
(b) adding a TLR4 agonist; and
(c) removing the solvent.
Clause 126. The method of manufacturing a liposomal adjuvant according to clause 122, comprising the following steps:
(a) mixing in the device a first solution comprising a solvent and lipid, and a second solution comprising water;
(b) removing the solvent; and
(c) adding a TLR4 agonist.
Clause 127. A method of manufacturing a liposomal concentrate of use in the preparation of a liposomal adjuvant using a microfluidic device according to any one of clauses 1 to 116, a chip according to either clause 117 or 119 or apparatus according to either clause 120 or 121, comprising the step of mixing in the device a first solution comprising a solvent and lipid, and a second solution comprising water.
Clause 128. The method of manufacturing a liposomal concentrate of use in the preparation of a liposomal adjuvant using a microfluidic device according to clause 127, comprising the following steps:
(a) mixing in the device a first solution comprising a solvent and lipid, and a second solution comprising water; and
(b) adding a TLR4 agonist.
Clause 129. The method of manufacturing a liposomal concentrate of use in the preparation of a liposomal adjuvant using a microfluidic device according to clause 127, comprising the following steps:
(a) mixing in the device a first solution comprising a solvent and lipid, and a second solution comprising water; and
(b) adding a saponin.
Clause 130. The method of manufacturing a liposomal concentrate of use in preparing a liposomal adjuvant using a microfluidic device according to any one of clauses 127 to 129, comprising the following steps:
(a) mixing in the device a first solution comprising a solvent and lipid, and a second solution comprising water;
(b) adding a saponin; and
(c) adding a TLR4 agonist;
wherein steps (b) and (c) may be in either order, or may be performed in a single step.
Clause 131. The method according to any one of clauses 122 to 130, wherein the first solution comprises a phosphatidylcholine lipid.
Clause 132. The method according to clause 131, wherein the first solution comprises DOPC.
Clause 133. The method according to any one of clause 122 to 132, wherein the first solution comprises a sterol.
Clause 134. The method according to any one of clause 122 to 124 or 127, 129 or 131 to 133, wherein the first solution comprises a TLR4 agonist.
Clause 135. The method according to any one of clause 122 to 124 or 131 to 133, wherein a TLR4 agonist is added before solvent removal.
Clause 136. The method according to any one of clause 122 to 124 or 131 to 133, wherein a TLR4 agonist is added after solvent removal.
Clause 137. The method according to any one of clause 122, 125 to 128 or 131 to 133, wherein the second solution comprises a saponin.
Clause 138. The method of any one of clauses 122 to 137, wherein the total flow rate into the mixing chamber is 12 to 40 ml/min/mm² of mixing chamber cross-section.
Clause 139. The method of clause 138, wherein the total flow rate into the mixing chamber is 17.5 to 25 ml/min/mm² of mixing chamber cross-section, in particular 19 to 21 ml/min/mm², such as 20 30 ml/min/mm².
Clause 140. The method of clause 138, wherein the total flow rate into the mixing chamber is 28 to 36 ml/min/mm² of mixing chamber cross-section, in particular 30 to 34 ml/min/mm², such as 32 ml/min/mm².
Clause 141. The method of any one of clauses 122 to 140, wherein the ratio of flow rates for the first and second solutions is in the range 1:2 to 1:6.
Clause 142. The method of clause 141, wherein the ratio of flow rates for the first and second solutions is in the range 1:3 to 1:5.
Clause 143. The method of clause 142, wherein the ratio of flow rates for the first and second solutions is 1:4.
Clause 144. The method of clause 141, wherein the ratio of flow rates for the first and second solutions is 1:2.5 to 1:3.5.
Clause 145. The method of clause 144, wherein the ratio of flow rates for the first and second solutions is 1:3.
Clause 146. The method of any one of clauses 122 to 145, wherein the flow rate of the first solution into the mixing chamber is 2 to 10 ml/min/mm² of mixing chamber cross-section.
Clause 147. The method of clause 146, wherein the flow rate of the first solution into the mixing chamber is 2 to 6 ml/min/mm², especially 3.5 to 5.5 ml/min/mm² and in particular 3 to 5 (e.g. 4) ml/min/mm² of mixing chamber cross-section.
Clause 148. The method of clause 147, wherein the flow rate of the first solution into the mixing chamber is 4.35 ml/min/mm².
Clause 149. The method of clause 146, wherein the flow rate of the first solution into the mixing chamber is 4.4 to 8.4 ml/min/mm², especially 4.9 to 6.9 ml/min/mm² and in particular 5.4 to 7.4 (e.g. 6.4) ml/min/mm² of mixing chamber cross-section.
Clause 150. The method of any one of clauses 122 to 149, wherein the flow rate of the second solution into the mixing chamber is 11 to 35 ml/min/mm² of mixing chamber cross-section.
Clause 151. The method of clause 150, wherein the flow rate of the second solution into the mixing chamber is 12 to 20 ml/min/mm², especially 14 to 18 ml/min/mm² and in particular 15 to 17 (e.g. 16) ml/min/mm² of mixing chamber cross-section.
Clause 152. The method of clause 150, wherein the flow rate of the second solution into the mixing chamber is 21.6 to 29.6 ml/min/mm², especially 23.6 to 27.6 ml/min/mm² and in particular 24.6 to 26.6 (e.g. 25.6) ml/min/mm² of mixing chamber cross-section.
Clause 153. The method of any one of clauses 122 to 149, wherein the flow rate of the second solution into the mixing chamber is 10 to 16 ml/min/mm², especially 11 to 15 ml/min/mm² and in particular 12 to 14 (e.g. 13.125) ml/min/mm².
Clause 154 The method of any one of clauses 122 to 153, wherein the first solution is provided at a temperature of 10 to 30° C.
Clause 155. The method of clause 154, wherein the temperature of the first solution is provided at a temperature of 15 to 25° C.
Clause 156. The method of any one of clauses 122 to 155, wherein the temperature of the second solution is provided at a temperature of 10 to 30° C.
Clause 157. The method of clause 156, wherein the temperature of the second solution is provided at a temperature of 15 to 25° C.
Clause 158. The method of any one of clauses 122 to 157, wherein the temperature of the mixing chamber is 10 to 30° C.
Clause 159. The method of clause 158, wherein the temperature of the mixing chamber is 15 to 25° C.
Clause 160. The method of any one of clauses 122 to 159, wherein the maximum Reynolds number within the mixing chamber is 1500 or lower.
Clause 161. The method of clause 160, wherein the maximum Reynolds number within the mixing chamber is 100 to 600, such as 150 to 500.
Clause 162. The method of any one of clauses 122 to 161, wherein the plurality of mixing chambers is capable of producing mixed material at a rate of 50 to 2000 ml/min, such as 200 to 500 ml/min.
Clause 163. The method of any one of clauses 122 to 162, wherein the plurality of mixing chambers is capable of producing mixed material at a rate of at least 1 g of lipid, such as 1 g of phosphatidylcholine lipid per minute.
Clause 164. The method of any one of clauses 122 to 163, wherein the plurality of mixing chambers is capable of producing mixed material at a rate of at least 1 g of DOPC per minute.
Clause 165. The method of any one of clauses 122 to 164, wherein the solvent comprises an organic alcohol.
Clause 166. The method of clause 165, wherein the solvent comprises ethanol.
Clause 167. The method of clause 166, wherein the solvent comprises 70 to 90% v/v ethanol. Clause 168. The method of clause 167, wherein the solvent comprises 75 to 85% v/v ethanol. Clause 169. The method of clause 168 wherein the solvent comprises 80% v/v ethanol.
Clause 170. The method according to any one of clauses 165 to 169, wherein the solvent comprises isopropanol.
Clause 171. The method of clause 170, wherein the solvent comprises 10 to 30% v/v isopropanol.
Clause 172. The method of clause 171, wherein the solvent comprises 15 to 25% v/v isopropanol.
Clause 173. The method of clause 172, wherein the solvent comprises 20% v/v isopropanol.
Clause 174. The method of any one of clauses 122 to 173, wherein the first solution comprises 100 to 170 mg/ml lipid, such as 100 to 170 mg/ml phosphatidylcholine lipid.
Clause 175. The method of clause 174, wherein the first solution comprises 100 to 160 mg/ml lipid, such as 100 to 160 mg/ml phosphatidylcholine lipid.
Clause 176. The method of clause 175, wherein the first solution comprises 130 mg/ml lipid, such as 130 mg/ml phosphatidylcholine lipid.
Clause 177. The method of any one of clauses 122 to 176, wherein the first solution comprises 100 to 170 mg/ml DOPC.
Clause 178. The method of clause 177, wherein the first solution comprises 100 to 160 mg/ml DOPC.
Clause 179. The method of clause 178, wherein the first solution comprises 130 mg/ml DOPC.
Clause 180. The method of any one of clauses 122 to 179, wherein the first solution comprises 20 to 50 mg/ml sterol.
Clause 181. The method of any one of clauses 122 to 180, wherein the first solution comprises 30 to 35 mg/ml sterol.
Clause 182. The method of any one of clauses 122 to 181, wherein the sterol is cholesterol.
Clause 183. The method of any one of clauses 122 to 182, wherein the dry weight of the first solution is 120 to 250 mg/ml.
Clause 184. The method of any one of clauses 122 to 183, wherein the second solution comprises at least 90% w/w water.
Clause 185. The method of clause 184, wherein the second solution comprises at least 98% w/w water.
Clause 186. The method of any one of clauses 122 to 185, wherein the saponin is Quil A or a derivative thereof.
Clause 187. The method of clause 186, wherein the saponin is QS-21.
Clause 188. The method of any one of clauses 122 to 187, wherein the second solution comprises 0.15 to 15 mg/ml saponin.
Clause 189. The method of clause 188, wherein the second solution comprises 1 to 4 mg/ml saponin.
Clause 190. The method of any one of clauses 122 to 189, wherein the TLR4 agonist is a lipopolysaccharide, such as a monophosphoryl lipid A.
Clause 191. The method of clause 190, wherein the lipopolysaccharide is 3D-MPL.
Clause 192. The method of clause 122 to 191, wherein the first solution comprises 4 to 10 mg/ml of the TLR4 agonist.
Clause 193. The method of any one of clauses 122 to 192, wherein the average liposome size is 95 to 120 nm.
Clause 194. The method of any one of clauses 122 to 192, wherein the average liposome size is 90 to 120 nm.
Clause 195. The method of any one of clauses 122 to 194, wherein the liposome polydispersity is 0.3 or lower, such as 0.25 or lower.
Clause 196. The method of clause 195, wherein the liposome polydispersity is 0.2 or lower.
Clause 197. The method of any one of clauses 122 to 196, wherein the solvent is removed by diafiltration, ultrafiltration and/or dialysis, in particular diafiltration.
Clause 198. The method of any one of clauses 122 to 197, wherein solvent removal results in a water content of at least 98% water w/w.
Clause 199. The method of any one of clauses 122 to 198, comprising the additional step of diluting, such as to a desired final concentration.
Clause 200. The method of any one of clauses 122 to 199, comprising the additional step of adjusting the pH to 5 to 9.
Clause 201. The method of any one of clauses 122 to 200, comprising the additional step of adjusting the osmolality to 250 to 750 mOsm/kg.
Clause 202. A method for the preparation of an adjuvanted immunogenic composition comprising an immunogen or antigen, or a polynucleotide encoding the immunogen or antigen, said method comprising the steps of: (i) manufacturing a liposomal adjuvant according to the method of any one of clauses 122 to 201;
(ii) mixing the liposomal adjuvant with an immunogen or antigen, or a polynucleotide encoding the immunogen or antigen.
Clause 203. A method for the manufacture of an adjuvanted immunogenic composition, said method comprising the step of combining an immunogen or antigen, or a polynucleotide encoding the immunogen or antigen, with a liposomal adjuvant manufactured according to the method of any one of clauses 122 to 201.
Clause 204. The method of any one of clauses 122 to 203, comprising the additional step of sterilisation by filtration.
Clause 205. A liposomal adjuvant comprising a saponin, TLR4 agonist, DOPC and sterol produced according to the method of any one of clauses 122 to 201.
Clause 206. An adjuvanted immunogenic composition produced according to the method of any one of clauses 202 to 204.
Clause 207. The adjuvant or immunogenic composition according to either clause 205 or 206 comprising saponin, such as QS-21, at an amount of 1 to 100 ug per human dose.
Clause 208. The adjuvant or immunogenic composition according any one of clauses 205 to 207 comprising TLR4 agonist, such as 3D-MPL, at an amount of 1 to 100 ug per human dose.
Clause 209. A liposome containing solution obtainable by, such as obtained by, mixing the first solution and second solution according to the methods of any one of clauses 122 to 201 or 204 prior to the removal of solvent.
Clause 210. The method, adjuvant, composition or solution according to any one of clauses 122 to 209, wherein the phosphatidylcholine lipid contains saturated unbranched acyl chains having 12 to 20 carbon atoms such as acyl chains having 14 to 18 carbon atoms.
Clause 211. The method, adjuvant, composition or solution according to any one of clauses 122 to 210, wherein the phosphatidylcholine lipid contains unbranched acyl chains having 12 to 20 carbon atoms and one double bond, such as acyl chains having 14 to 18 carbon atoms and one double bond.
Clause 212. The method, adjuvant, composition or solution according to any one of clauses 122 to 211, wherein the TLR4 agonist is dLOS.
Clause 213. The method, adjuvant, composition or solution according to any one of clauses 122 to 212, wherein the lipid comprises DMPC.
Clause 214. The method, adjuvant, composition or solution according to any one of clauses 122 to 213, wherein the lipid comprises DOTAP.
Clause 215. The method, adjuvant, composition or solution according to any one of clauses 122 to 214, wherein the ratio of saponin:lipid is 1:50 to 1:10 (w/w), suitably between 1:25 to 1:15 (w/w), and preferably 1:22 to 1:18 (w/w), such as 1:20 (w/w).
Clause 216. The method, adjuvant, composition or solution according to any one of clauses 122 to 215, wherein the ratio of saponin:DOPC is 1:50 to 1:10 (w/w), suitably between 1:25 to 1:15 (w/w), and preferably 1:22 to 1:18 (w/w), such as 1:20 (w/w).
Clause 217. The method, adjuvant, composition or solution according to any one of clauses 122 to 216, wherein the ratio of DOPC:sterol, such as cholesterol, is 10:1 to 1:1 (w/w), suitably between 8:1 to 2:1 (w/w), and preferably 6:1 to 2.6:1 (w/w), such as about 4:1 (w/w).

The invention will be further described by reference to the following, non-limiting, examples:

### EXAMPLES

### Example 1

WO2018219521 discloses a microfluidic device comprising a serpentine central/internal channel. The aim of the serpentine topography was to ensure that the overall length of the central/internal channel was substantially the same as the overall length of the outer/external channels.

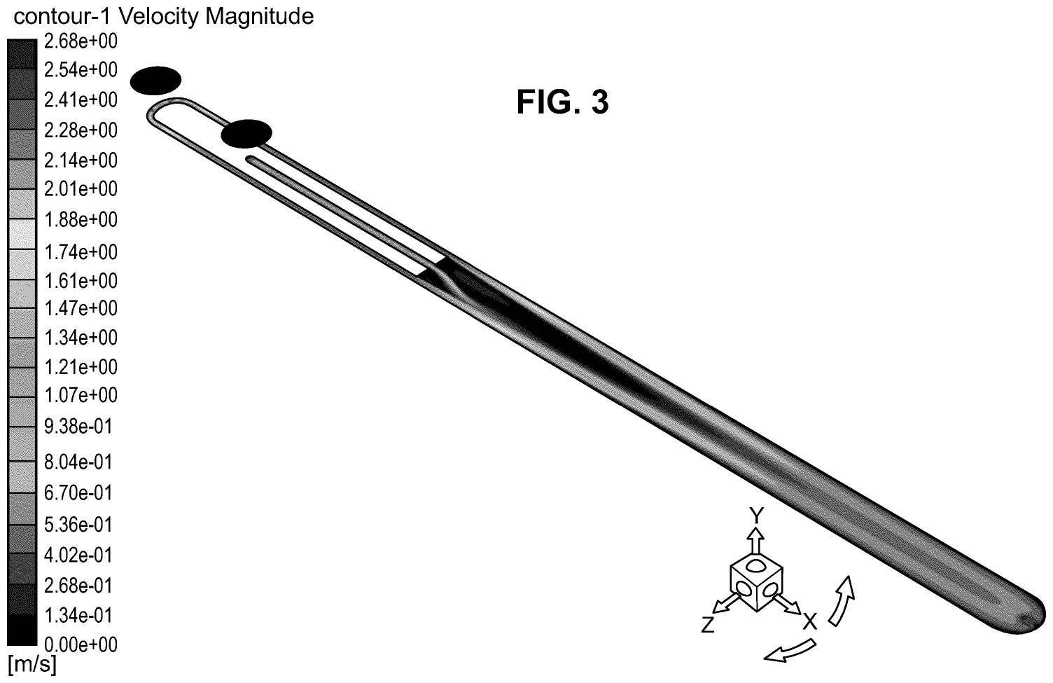

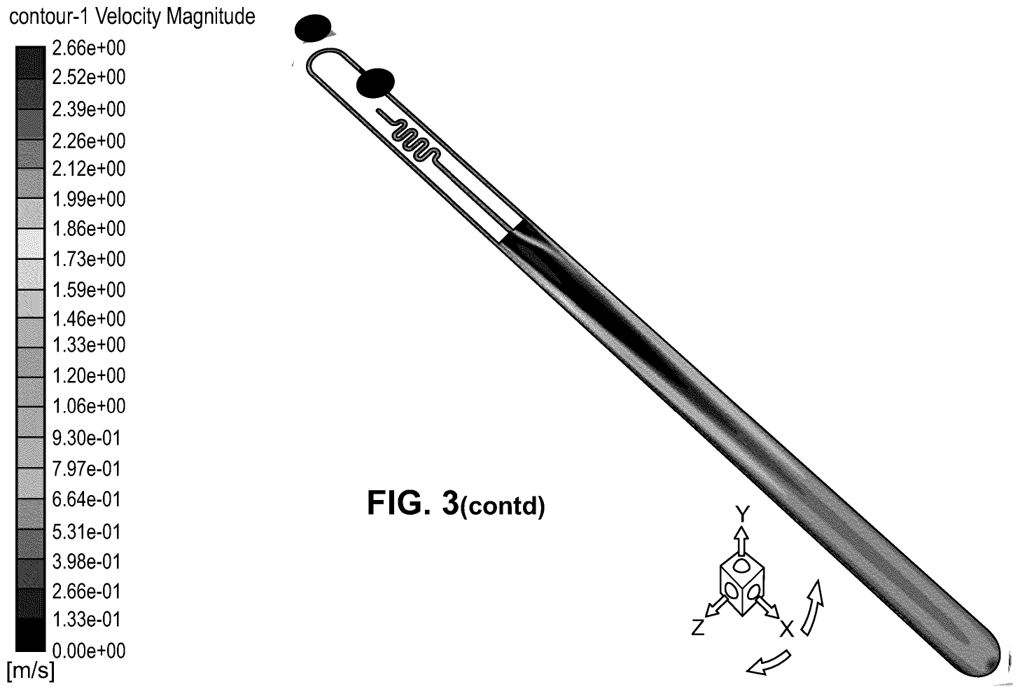

Computational fluid dynamics simulations were performed to investigate the impact of the central channel on fluid flow and to determine if a serpentine channel was beneficial. FIG. 3 shows that replacing the serpentine central channel (lower panel) with a linear central channel (upper panel) has no significant impact on fluid flow or mixing. The use of a linear central channel is advantageous for manufacturing.

### Example 2

Six microfluidic devices were prepared to investigate the effect of modifying channel width and mixing chamber width. Design 1 corresponds to the arrangement presented in WO2018219521. One of the devices (Design 6) was modified to replace the conical inlet and outlet holes with cylindrical inlets and outlets:

| | | | | Mixing | | |
|---|---|---|---|---|---|---|
| | External | Internal | Mixing | Chamber | | Serpentine |
| | Channel | Channel | Chamber | Length | Channel | Internal |
| Design No. | Width (mm) | Width (mm) | Width (mm) | (mm) | Depth (mm) | Channel |
| 1 | 0.2 | 0.2 | 2 | 25 | 0.4 | YES |
| 2 | 0.2 | 0.2 | 2 | 25 | 0.4 | NO |
| 3 | 0.2 | 0.4 | 2 | 25 | 0.4 | NO |
| 4 | 0.4 | 0.2 | 2 | 25 | 0.4 | NO |
| 5 | 0.2 | 0.2 | 1.6 | 17.5 | 0.4 | NO |
| 6 | 0.2 | 0.2 | 2 | 25 | 0.4 | YES |

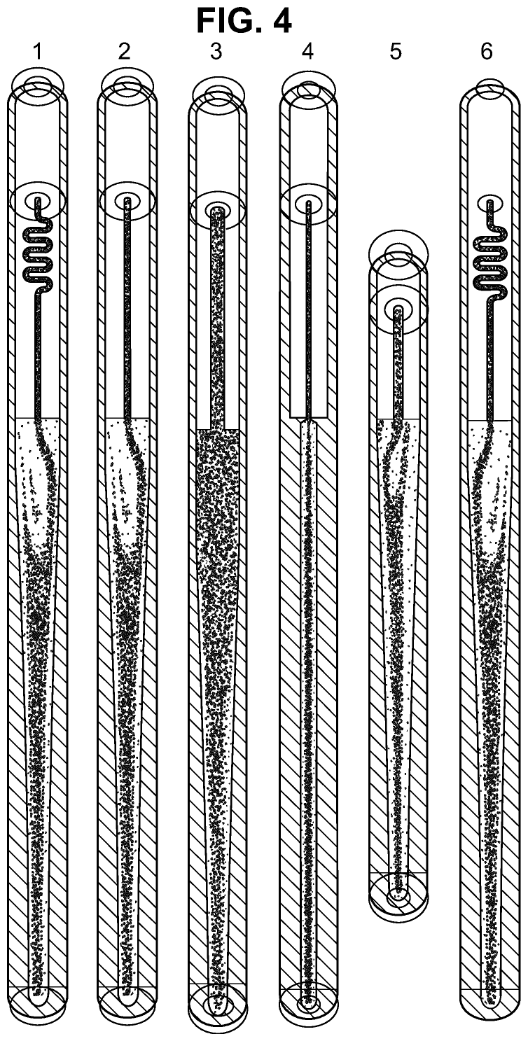

FIG. 4 shows the results of the computation fluid dynamics (CFD) simulations for each of the designs using the same flow rate and ratio (total 16 ml/min, 4:1 External/Internal channels).

As previously observed, the presence of the serpentine in the central capillary does not affect the profile compared to the same design without this serpentine (comparing Designs 1 and 2).

Increasing the width of the external channels (comparing Designs 2 and 4) resulted in a narrow distribution of the model dye along the length, indicating that mixing was low.

Increasing the width of the central channel (comparing Designs 2 and 3) resulted in a flow profile that was broader and more homogenous.

Changing the inlet and outlet to a cylindrical shape appeared to have little effect on the flow profile (comparing Designs 1 and 6). Similarly, the design having a reduced mixing chamberwidth and length (Design 5) exhibited a flow profile similar to Designs 1 and 2.

In order to compare mixing, Equation 1 (based on equations 5 and 6 of Javid, 2018) was used to determine mixing performance for each design:

$$α = 1 - \sqrt{\frac{{σ}^{2}}{{σ}_{max}^{2}}}$$

$$with$$

$${σ}^{2} = \frac{1}{n} {\sum }_{i = 1}^{n} {( {C}_{i} - \overline{C} )}^{2} ,$$

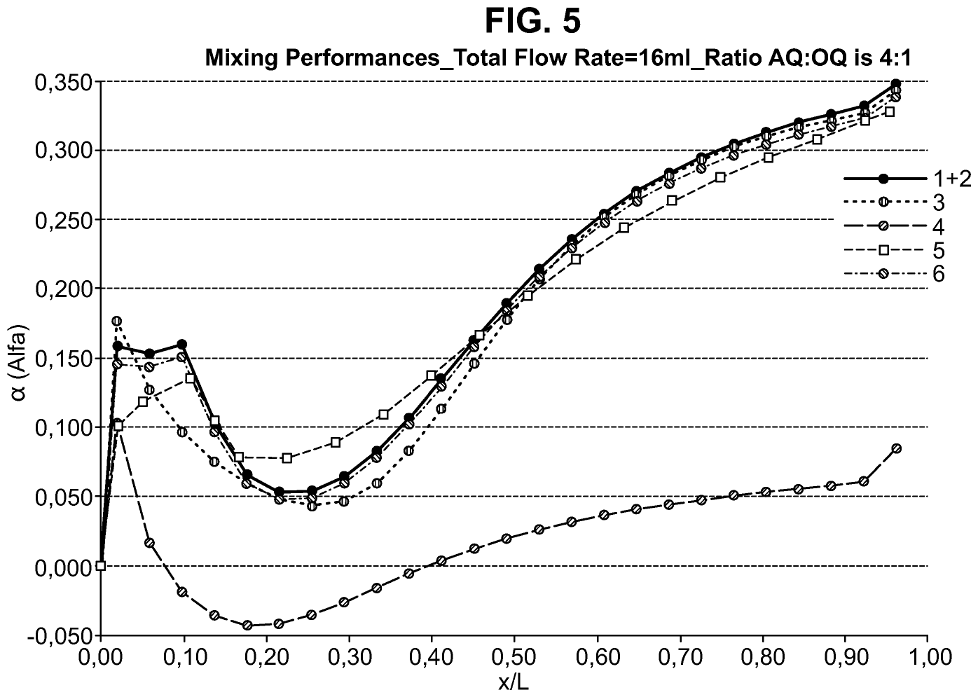

FIG. 5 shows a comparison of the mixing performance for each design (note that the lines for Designs 1 and 2 overlay precisely). The x axis corresponds to the position along the length of the mixing chamber as a proportion of total chamber length to enable the different designs to be compared.

These results confirmed the findings of the CFD simulations showing that increasing the width of the external channels resulted in reduced mixing of fluids entering the mixing chamber.

### Example 3

Based on the results obtained above, a further series of experiments was undertaken to determine which dimensions of the geometry had the most impact on mixing performance. In these experiments, the width of the mixing chamber (MC) was either 1 mm, 2 mm or 3 mm; the width of the internal linear channel (Capint) was either 0.1 mm, 0.2 mm or 0.3 mm; the width of the external channels (CapExt) was either 0.1 mm, 0.2 mm or 0.3 mm.

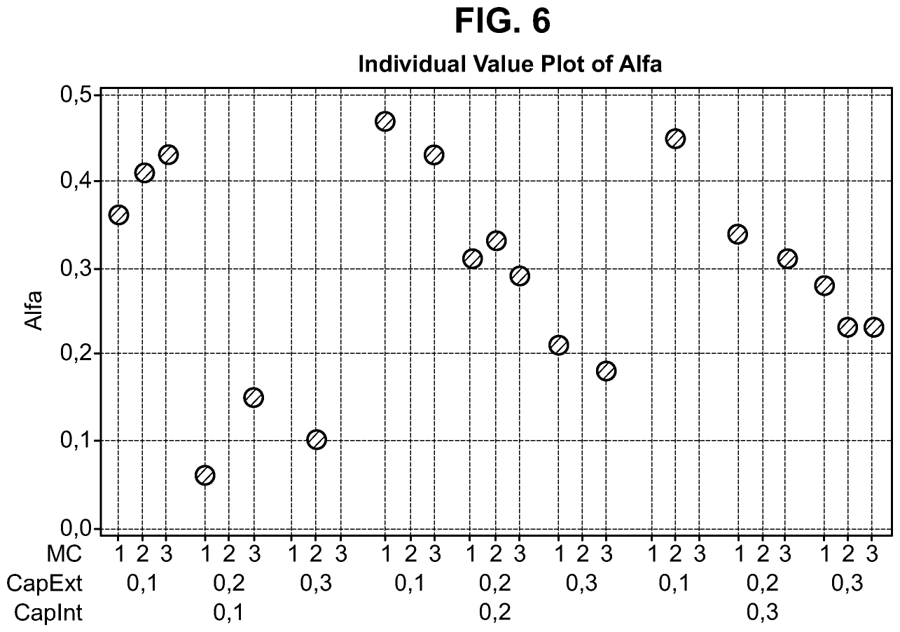

A final mixing coefficient (Alpha) was determined for each of these 19 different designs (FIG. 6). The highest value of alpha, i.e. best mixing performance, was obtained using a microfluidic device having a mixing chamber width of 1 mm, external channel width of 0.1 mm and internal channel width of 0.2 mm.

Surprisingly, mixing performance appeared to be mainly driven by the width of the external channels; mixing chamber width seemed to have less impact on the mixing.

### Example 4

The following model using only significant terms for mixing performance (alpha) was extracted:

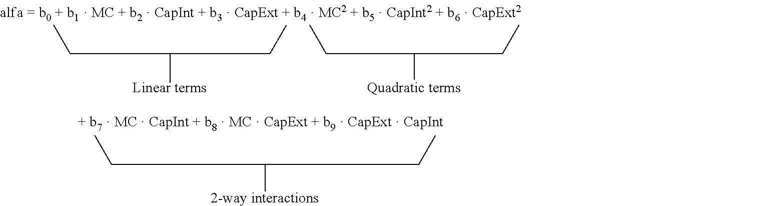

The model was used to determine geometries giving the best mixing coefficient using a stepwise or forward procedure.

The following table summarizes the best dimensions, in millimetres (mm), for mixing:

| | MC | CapInt | CapExt | Alfa Predicted |
|---|---|---|---|---|
| Stepwise model | 1 | 0, 27 | 0, 1 | 0, 5 ± 0, 1 |
| Forward model | 1, 8 | 0, 23 | 0, 1 | 0, 5 ± 0, 1 |
| Best Bubble (best simulation) | 1 | 0, 2 | 0, 1 | 0, 47 |

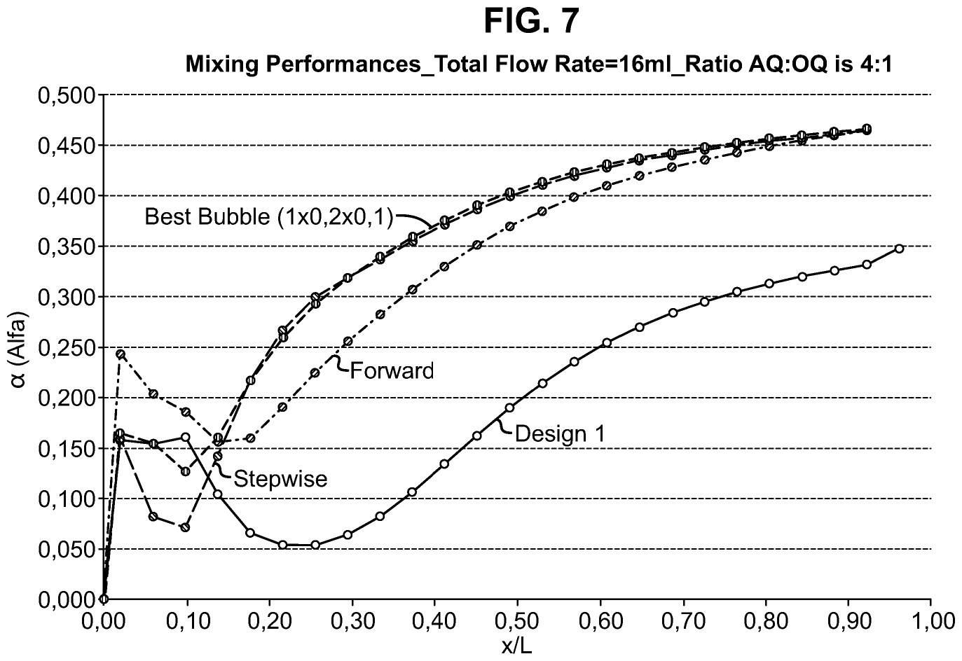

FIG. 7 shows a comparison of the mixing profile of the modified geometry versus Design 1. An increased mixing index has been achieved for the modified geometries compared to Design 1 (from WO2018219521).

### Example 5

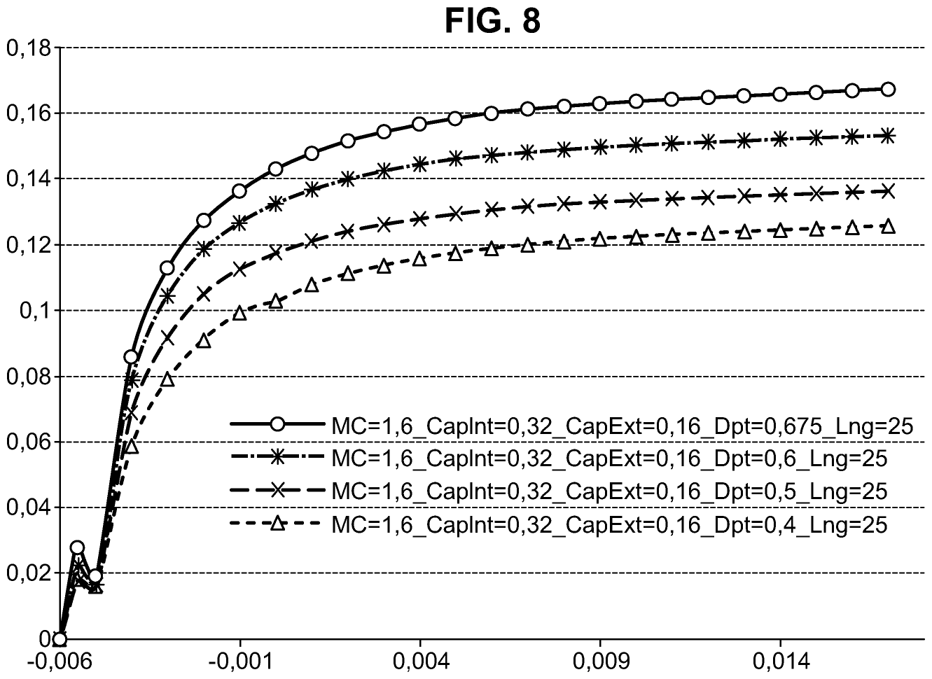

The impact of channel depth was investigated (FIG. 8). Simulations were performed (based on equations 6, 7 and 8 of Galletti, 2012) using the same geometry but varying the depth of the channel:

$$\overline{c} = \frac{〈 cv 〉}{〈 v 〉} ;$$

$$〈 cv 〉 = \frac{1}{A} {\int }_{A} cvdA ;$$

$$〈 v 〉 = \frac{1}{A} {\int }_{A} vdA$$

$$\overline{{( Δ c )}^{2}} = \frac{〈 {( c - \overline{c} )}^{2} v 〉}{〈 v 〉} ;$$

$${σ}_{c m}^{2} = \frac{\overline{{( Δ c )}^{2}}}{\overline{{( Δ c )}_{max}^{2}}}$$

$${δ}_{m} = 1 - {σ}_{c m}$$

Increases in channel depth appeared to improve mixing efficiency. However, the depth of the channels is dependent on the thickness of the substrate, in this case the silicon wafer. For a wafer 675 um thick, the maximum depth should not be deeper than 500 um.

### Example 6

Microfluidic chips of the following dimensions were produced with two depths (400 um and 500 um):

| | Width | Width | Width | Length |
|---|---|---|---|---|
| | mixing | External | Internal | mixing |
| | chamber | channels | channel | chamber |
| Design | (mm) | (mm) | (mm) | (mm) |
| 1 | 2 | 0.2 | 0.4 | 25 |
| 2 | 2 | 0.4 | 0.2 | 25 |
| 3 | 1.6 | 0.2 | 0.4 | 25 |
| 4 | 1 | 0.1 | 0.27 | 25 |
| 5 | 1.8 | 0.1 | 0.23 | 25 |
| 6 | 1.6 | 0.15 | 0.2 | 25 |
| 8 | 1.6 | 0.2 | 0.4 | 50 |
| 12 | 1.6 | 0.4 | 0.2 | 50 |
| 14 | 1.6 | 0.4 | 0.7 | 25 |
| 15 | 1.6 (Start from | 0.4 | 0.7 | 25 |
| | 1.6 end at 0.75) | | | |

First solution (organic phase) was prepared essentially as described in WO2018219521. Ethanol/isopropanol 80:20 was used to prepare a final solution containing 3D-MPL 6.5 mg/ml, DOPC 130 mg/ml and cholesterol 32.5 mg/ml. Second solution (aqueous phase) was prepared by diluting concentrated QS-21 stock solution with water for injection to achieve a final concentration of 1.625 mg/ml. The devices were operated at 20 degrees with a total flow rate of 16 ml/min (flow rate ratio 4:1 External/Internal channels).

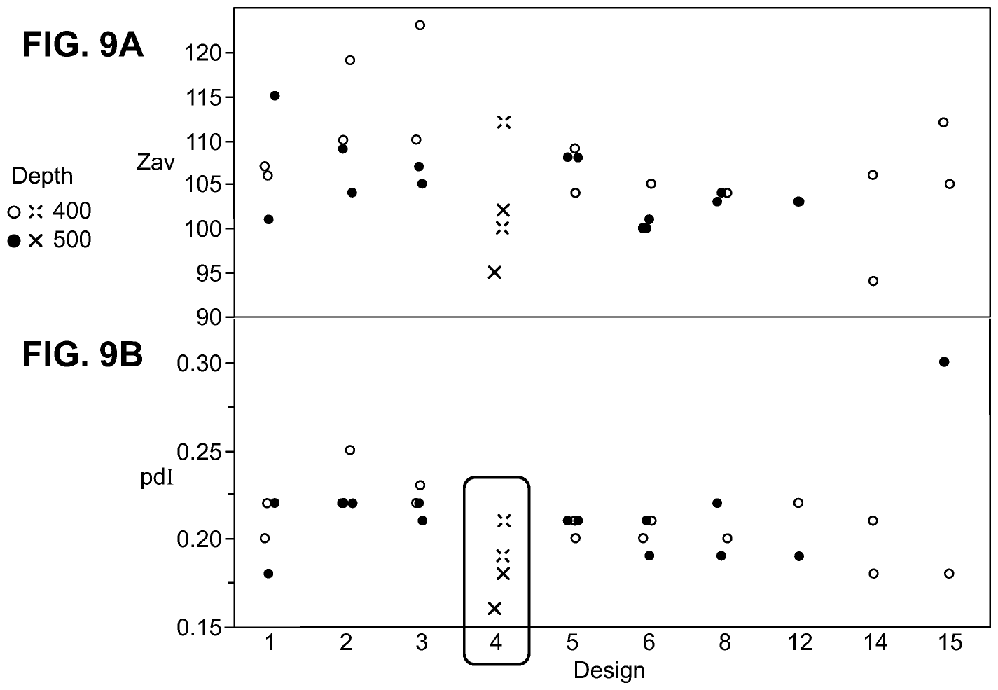

Sizes were determined using Malvern Zeta Nano series. Samples were diluted to obtain consistent concentration for measurement (ca 2 mg/ml DOPC) but without further processing such as solvent removal. The results (size and PDI) from duplicate experiments were plotted for each design, (FIG. 9). Design 4 demonstrated a lower PDI when the depth is at 500 um.

This design was therefore selected for further experiments assessing the impact of temperature and flow rate (total flow rate 15 to 19 ml/min, temperature 16 to 22° C.). Other parameters remained constant (stock concentration, ratio aqueous/organic phases 4:1). Conditions minimizing the size and polydispersity were temperature around 19 to 20° C. and a flow rate at 16 ml/min. However, while an improvement was observed compared with the design of WO2018219521, the improvement observed was less than desired (data not shown).

### Example 7—Modelling the Impact of Different Baffle Arrangements on Mixing

In order to investigate the impact of baffles and determine the optimum in terms of baffle position, size and shape, a CFD study was performed.

Some parameters were fixed such as mixing chamber width (1.6 mm), mixing chamber length (25 mm) and total depth (500 um). Those values were chosen following the results obtained in earlier work and also to facilitate future integration with 16 mixing chambers in parallel (a mixing chamber width above 1.6 mm will be difficult to integrate high numbers of chambers into the defined space which is limited by the selected manufacturing approach) as well as minimizing pressure issues. Two geometries were chosen as a starting point (i.e. Designs 3 and 6 from Example 6, with a depth of 500 um).

Investigated parameters were sequentially tested as highlighted as most critical (FIG. 10):
1) Impact of baffle numbers and starting position
2) Impact of baffle position (alignment, distance between 2 baffles)
3) Baffle dimensions
4) Baffle shape (square, trapezoid, bell, wave)

Selection criteria were set:
1) Mixing efficiency >0.8 end of mixing chamber
2) Minimal pressure drops (˜1 bar)
3) Manufacturability (obstacles dimensions, process tolerance vs dimensions)
4) No dead zones (material accumulation, air bubbles entrapment . . . )

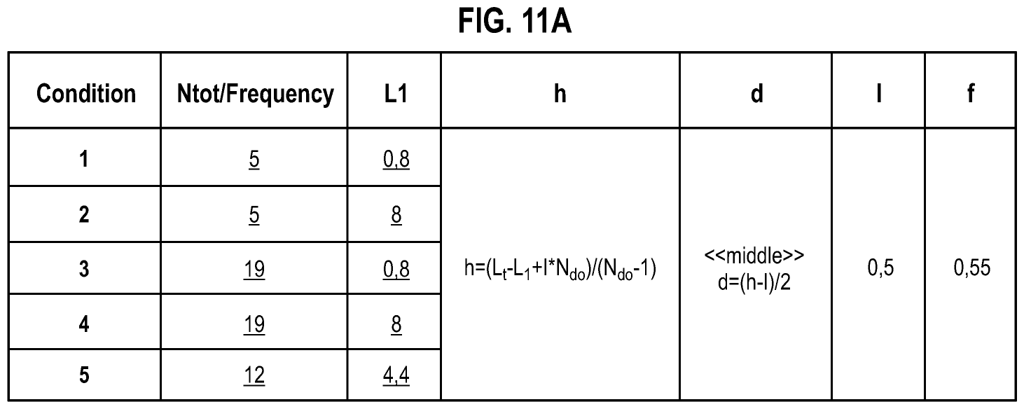

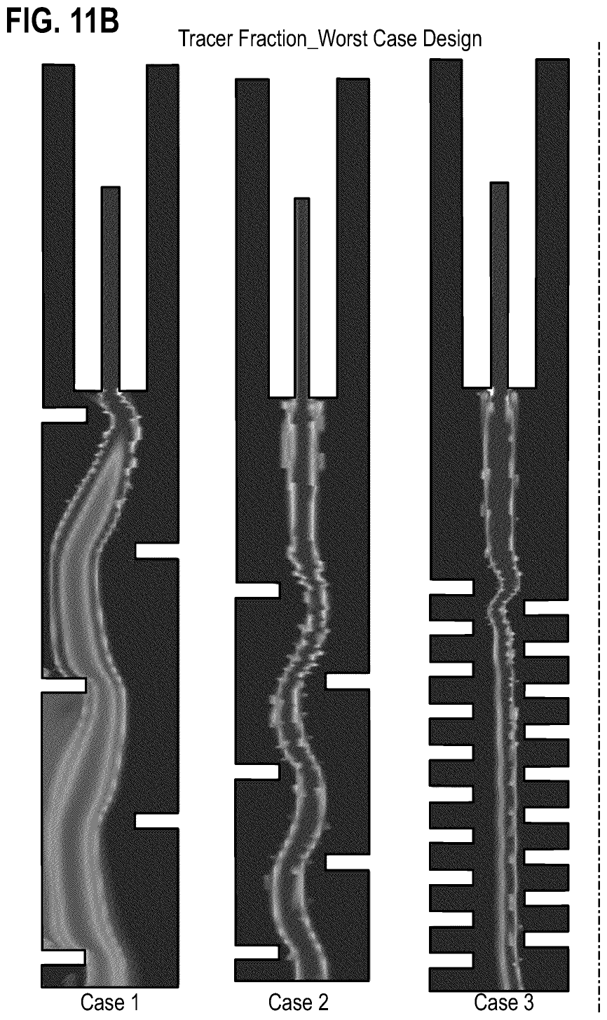

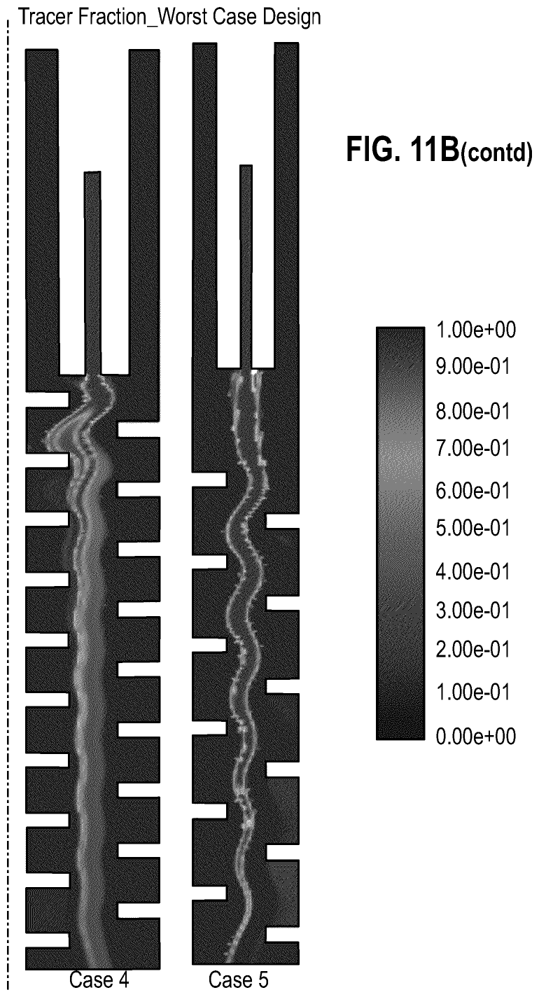

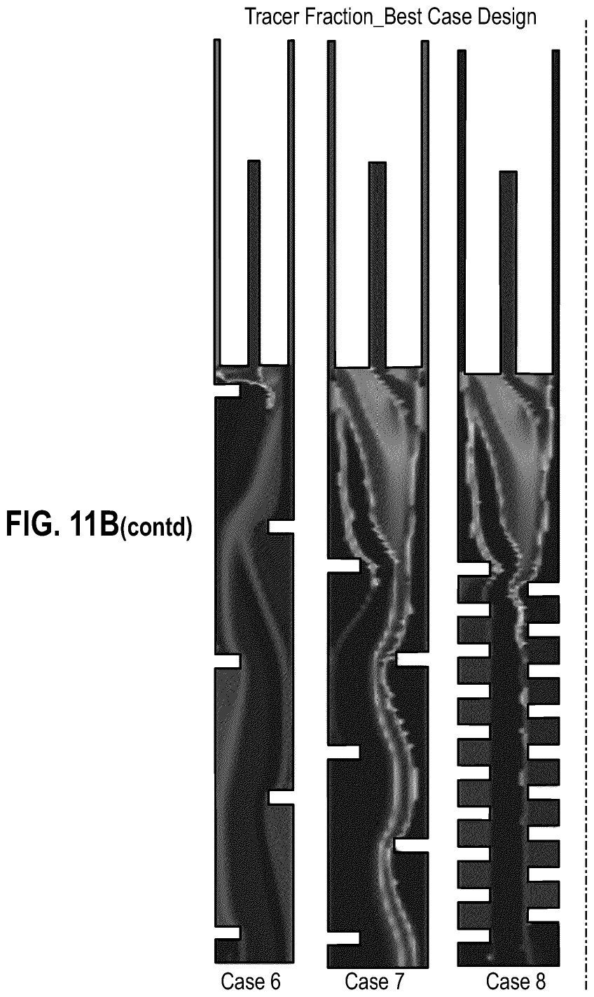

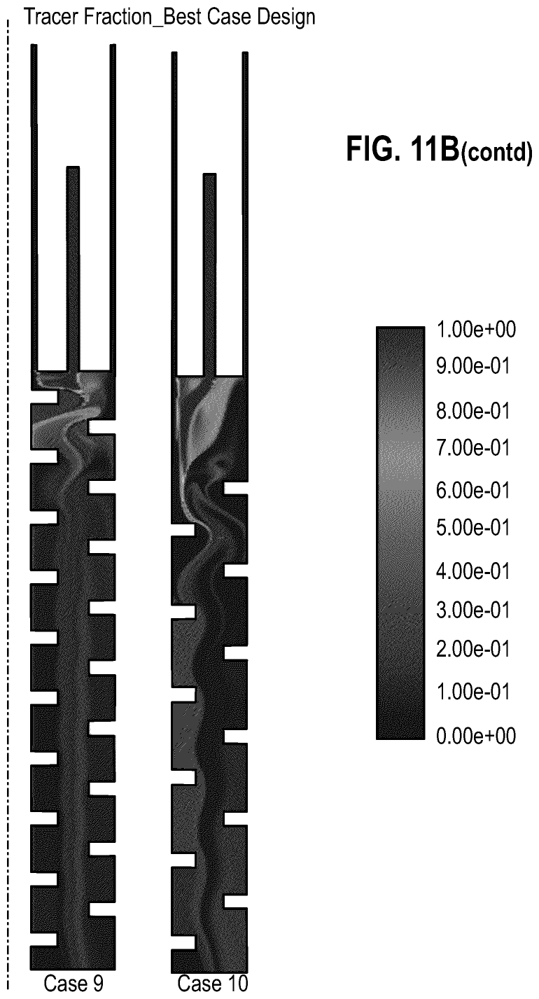

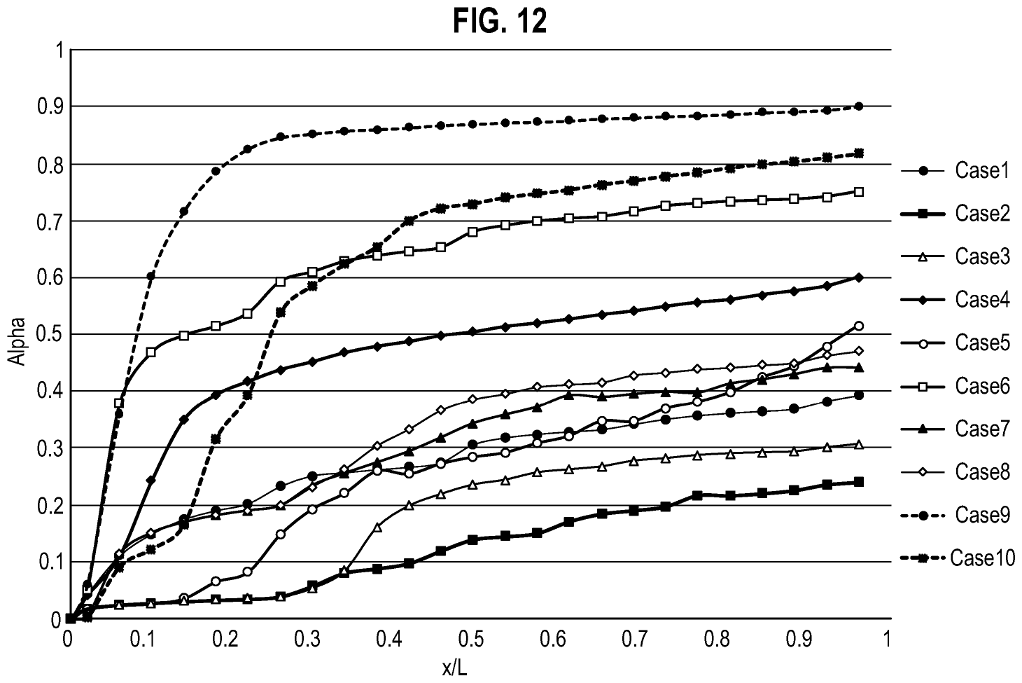

The conditions described in FIG. 11A were tested (Nₜₒₜ/frequency indicating the total number of baffles) and FIG. 11B shows the results by mass fraction distribution of a model dye for Cases 1 to 10 (Cases 1 to bases on Conditions 1 to 5 and Design 3 from Example 6; Cases 6 to 10 based on Conditions 1 to 5 and Design 6 from Example 6). FIG. 12 plots the mixing efficiency for the different designs modelled.

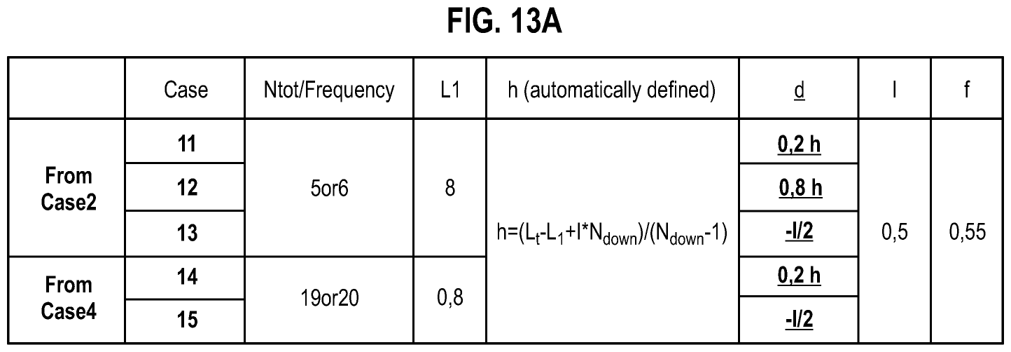

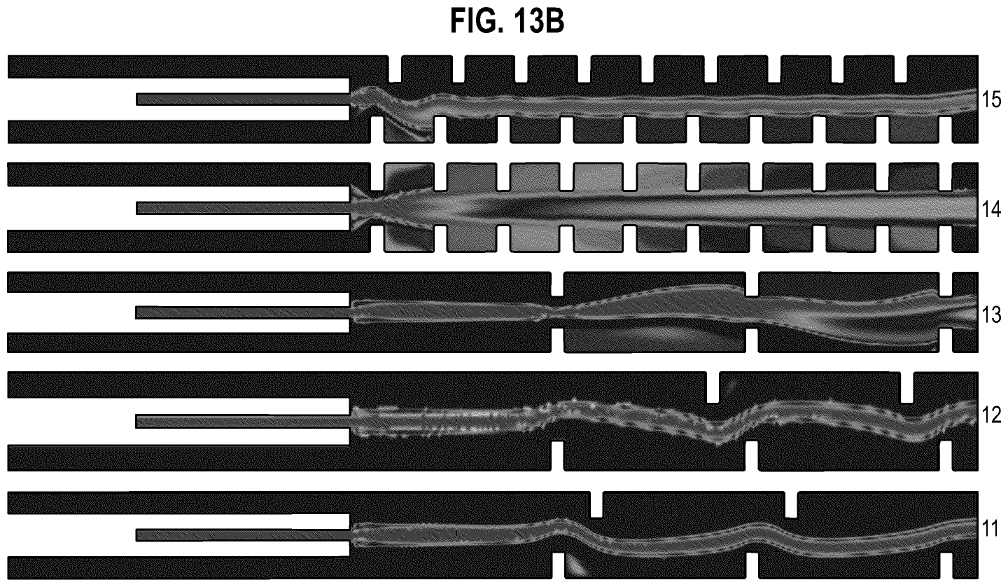

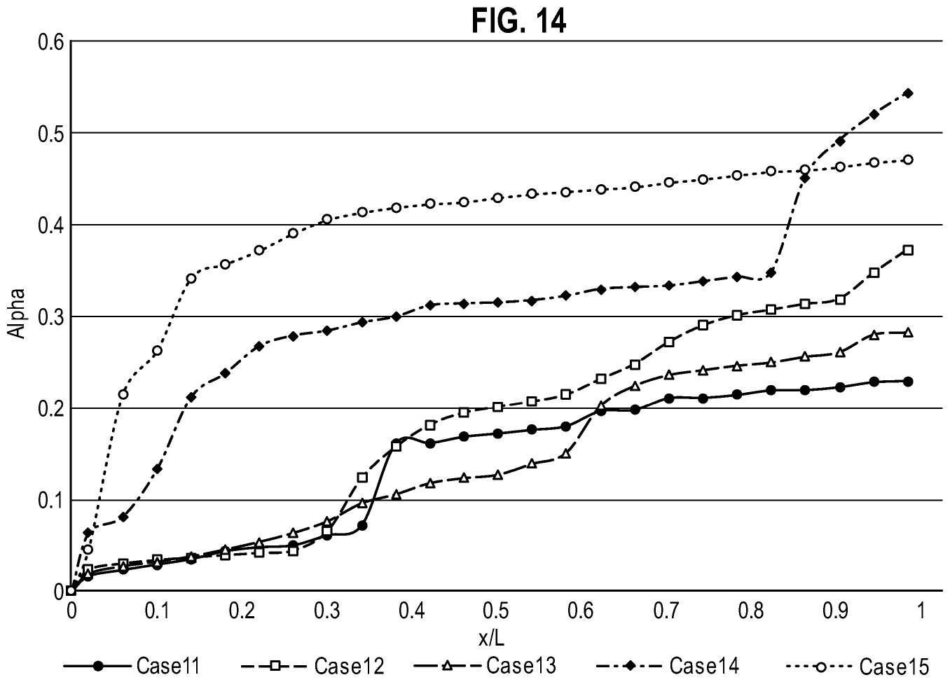

Cases 2 (worst case) and 4 (‘best of worst’) were further modified as described in FIG. 13A and FIG. 13B shows the results by mass fraction distribution of a model dye for Cases 11 to 15. FIG. 14 plots the mixing efficiency for the different designs modelled.

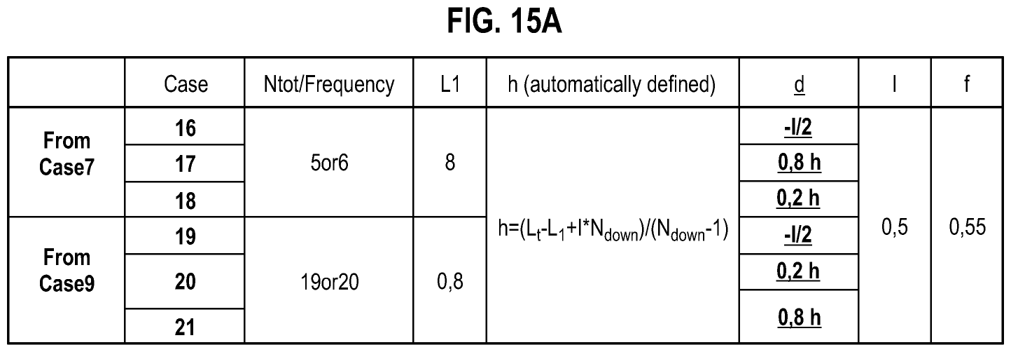

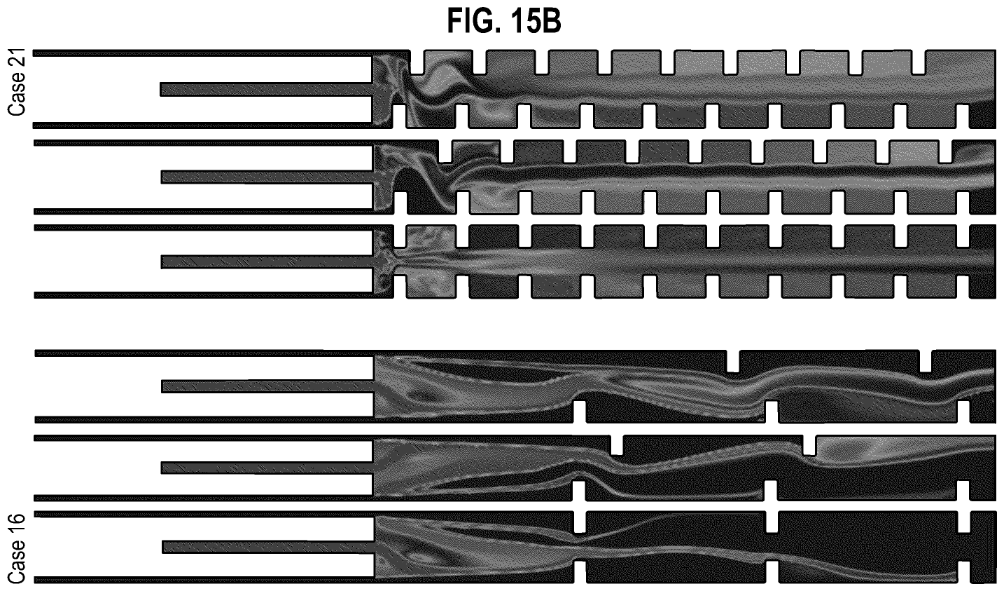

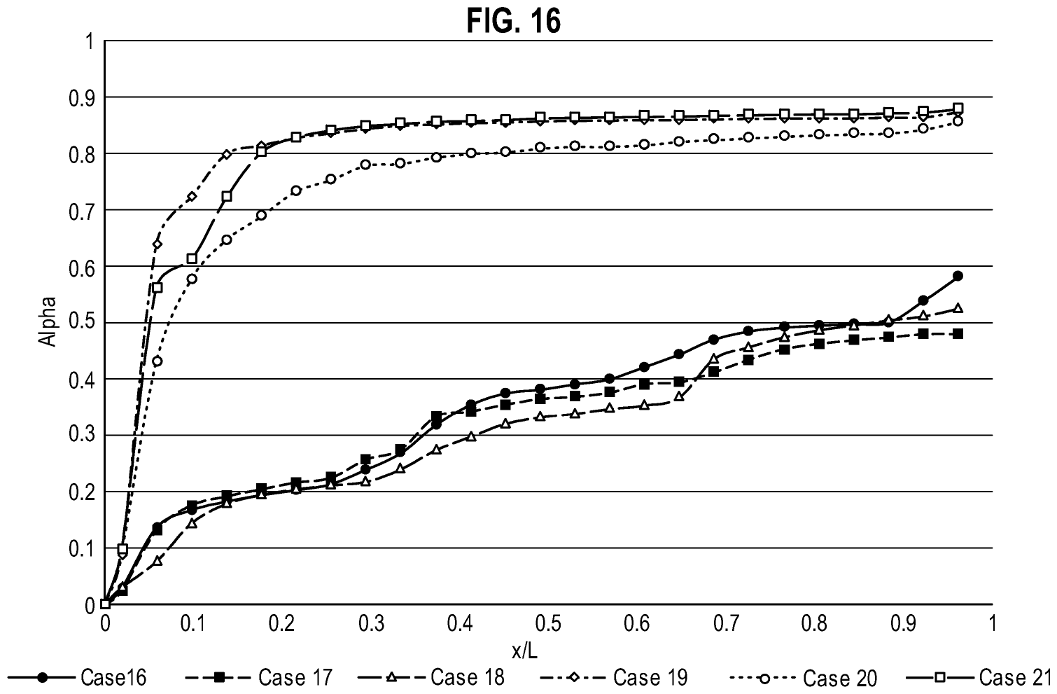

Cases 9 (best case) and 7 (‘worst of best’) were further modified as described in FIG. 15A and FIG. 15B shows the results by mass fraction distribution of a model dye for Cases 16 to 21. FIG. 16 plots the mixing efficiency for the different designs modelled. Cases 19, 20 and 21 performed particularly well.

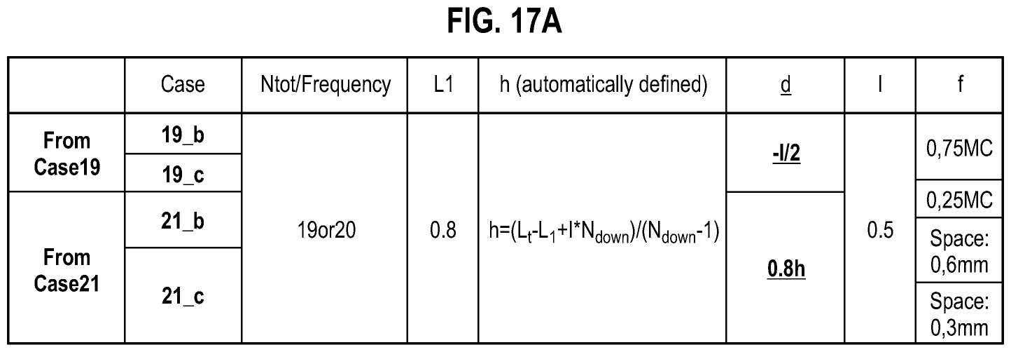

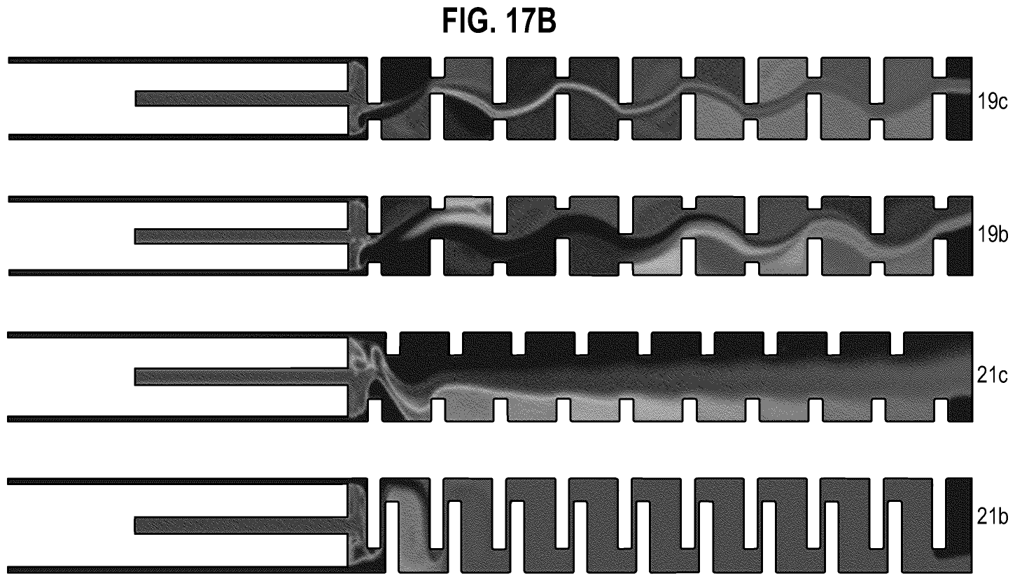

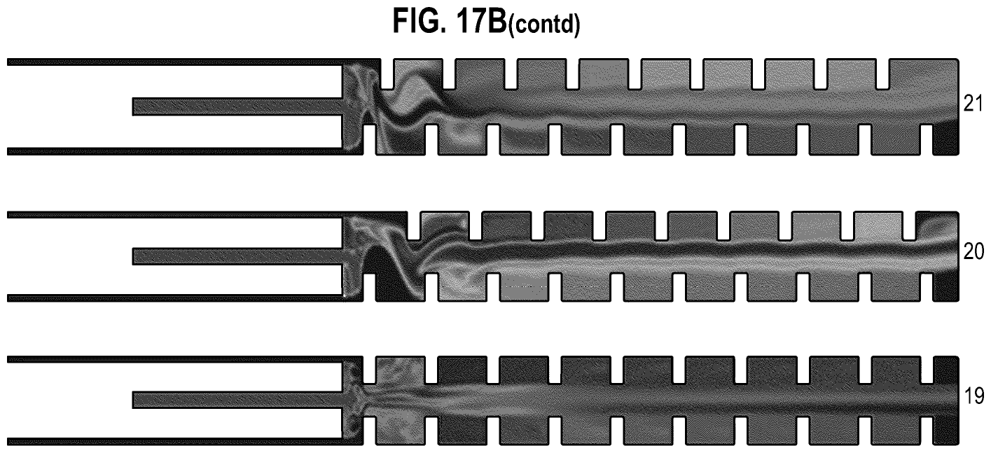

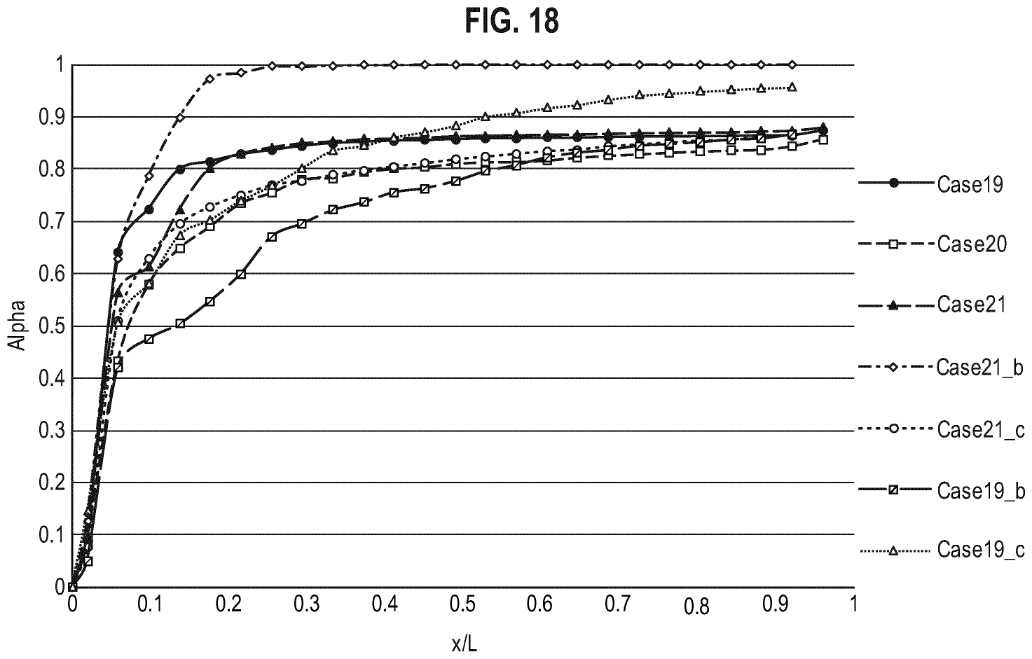

Cases 19 and 21 were further modified as described in FIG. 17A and FIG. 17B shows the results by mass fraction distribution of a model dye for Cases 19b, 19c, 21b and 21c. FIG. 18 plots the mixing efficiency for the different designs modelled, along with Cases 19 to 21 for comparison.

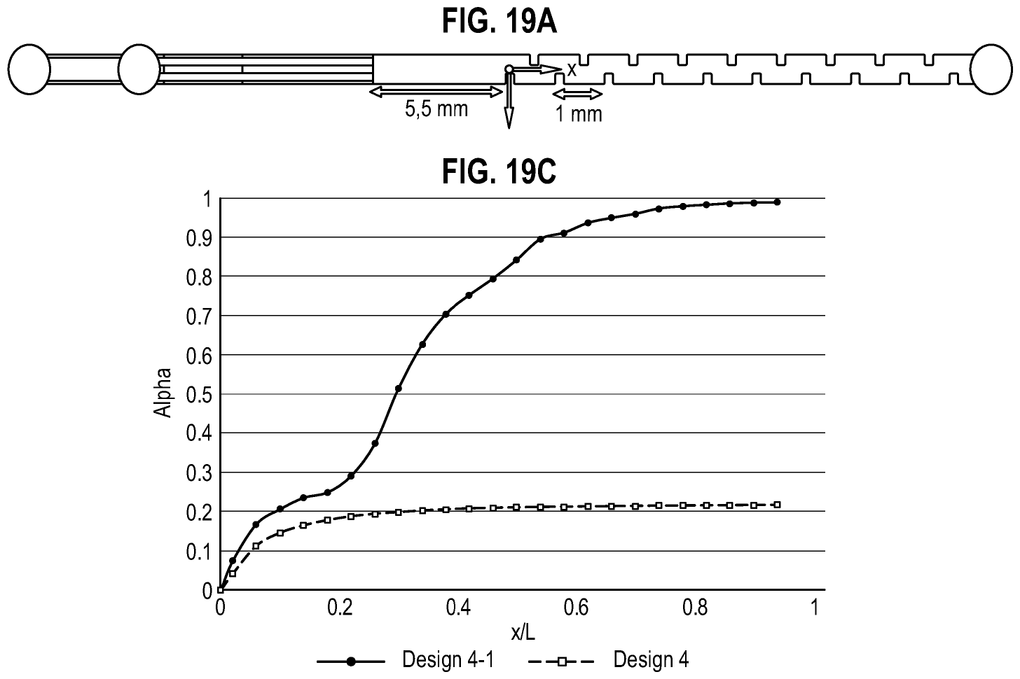

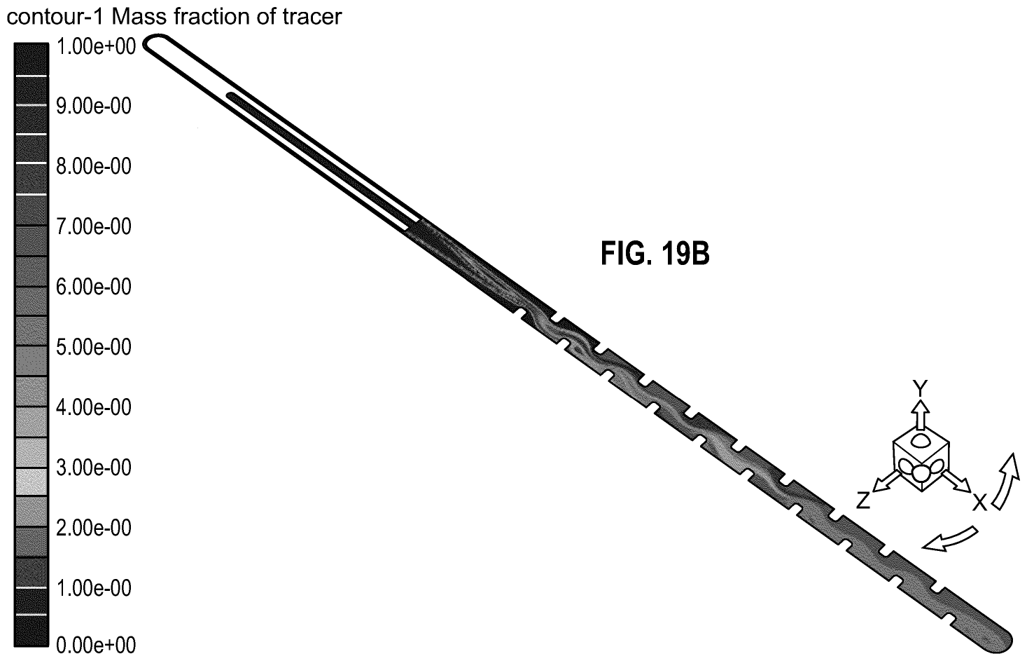

A further geometry was chosen as a starting point (i.e. Design 4 from Example 6, with a depth of 500 um) and modified by the introduction of baffles as described in FIG. 19A (referred to as Design 4-1). FIG. 19B shows the results by mass fraction distribution of a model dye for Design 4-1. FIG. 19C plots the mixing efficiency as compared to Design 4.

### Example 8—Modelling the Impact of Different Baffle Shapes on Mixing

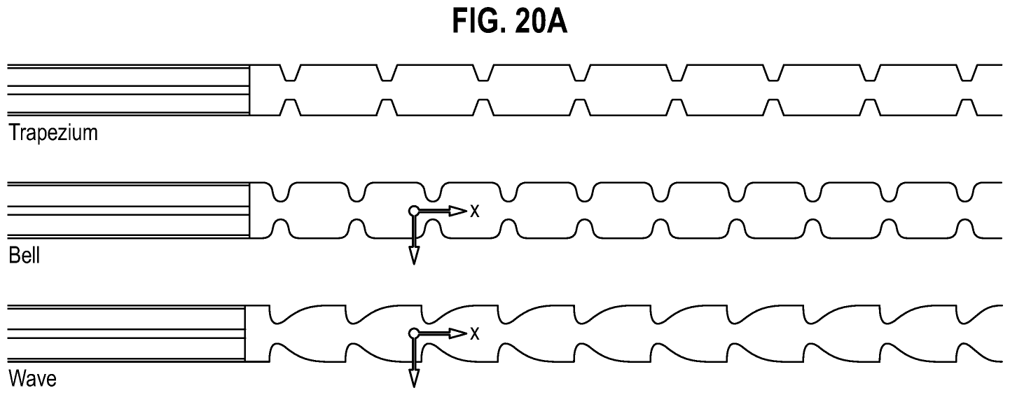

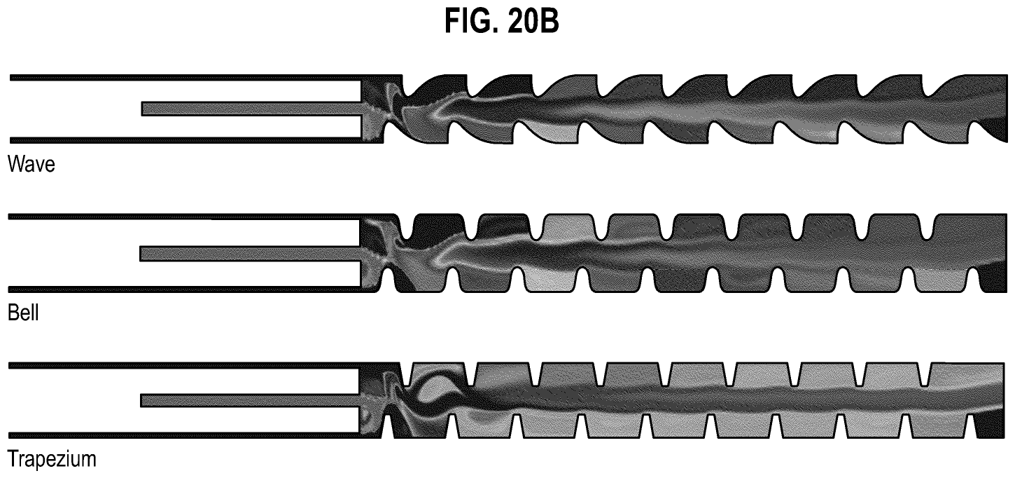

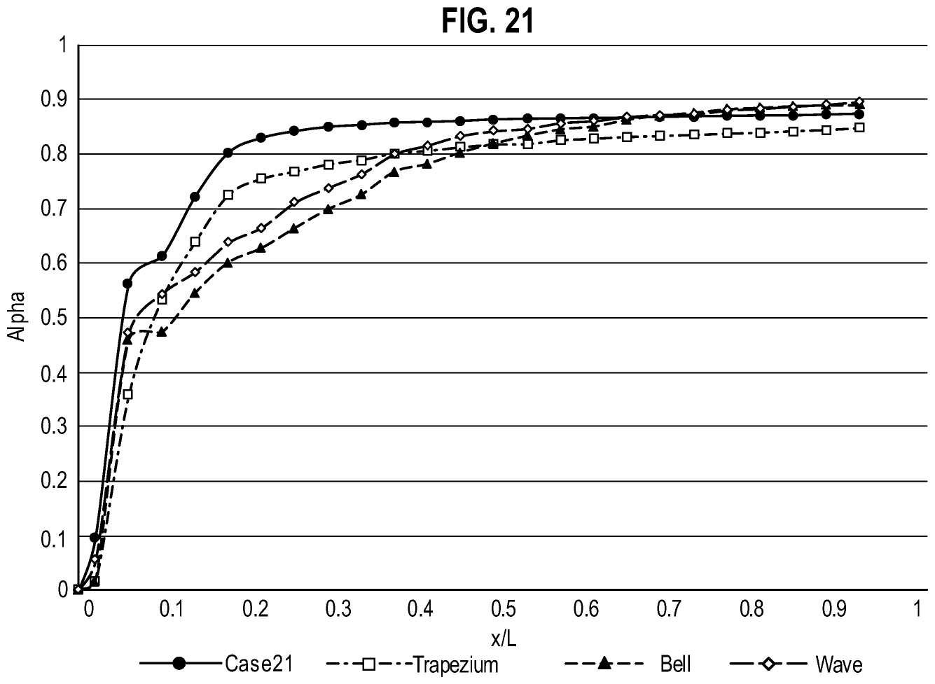

Case 21 from Example 7 utilised baffles with a rectangular profile. The impact of different baffle shapes on mixing was investigated by replacing the rectangular profile baffles with trapezium, bell or wave profiles as shown in FIG. 20A. Computational fluid dynamics simulation of the new baffle shapes is shown in FIG. 20B. FIG. 21 presents a comparison of the mixing profile for the new trapezium, bell or wave profiles compared to the original rectangular profile. Baffle profile was found to have a limited impact on the mixing efficiency.

### Example 9—Testing of Baffled Designs in the Manufacture of Liposomal Adjuvant

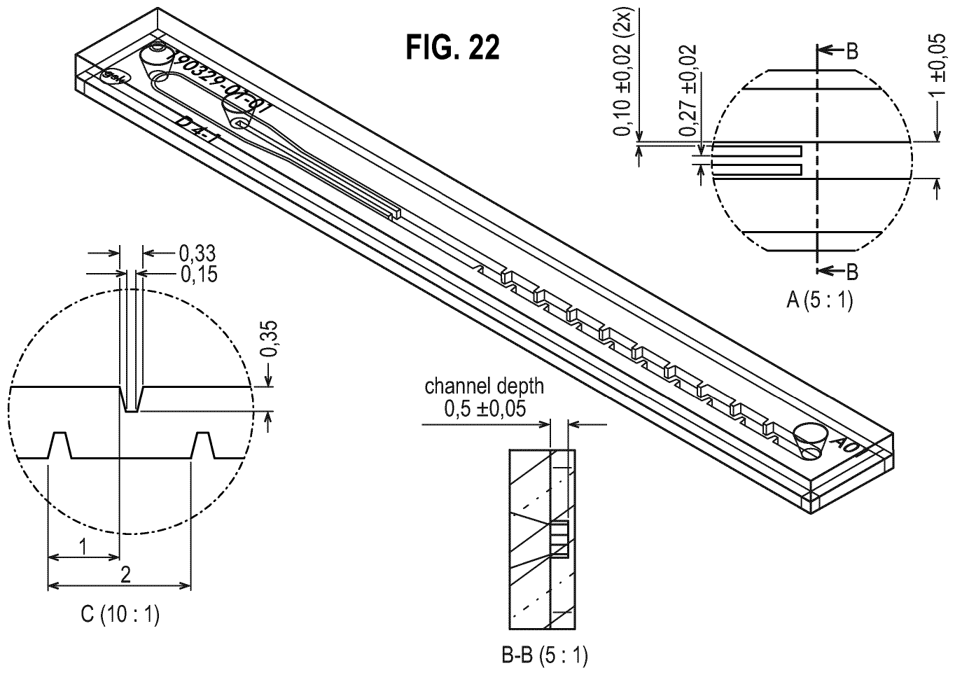

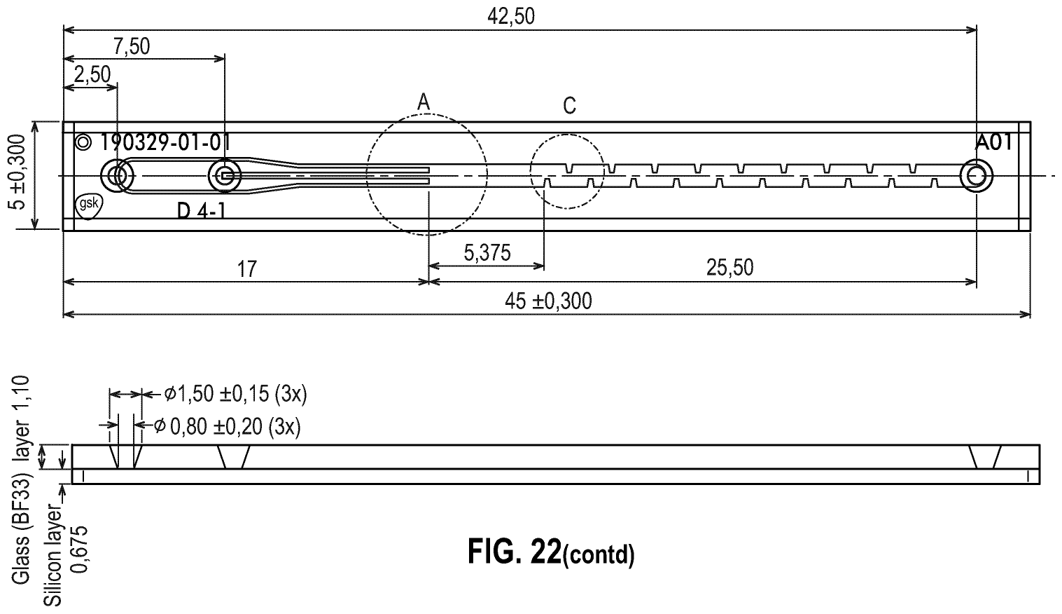

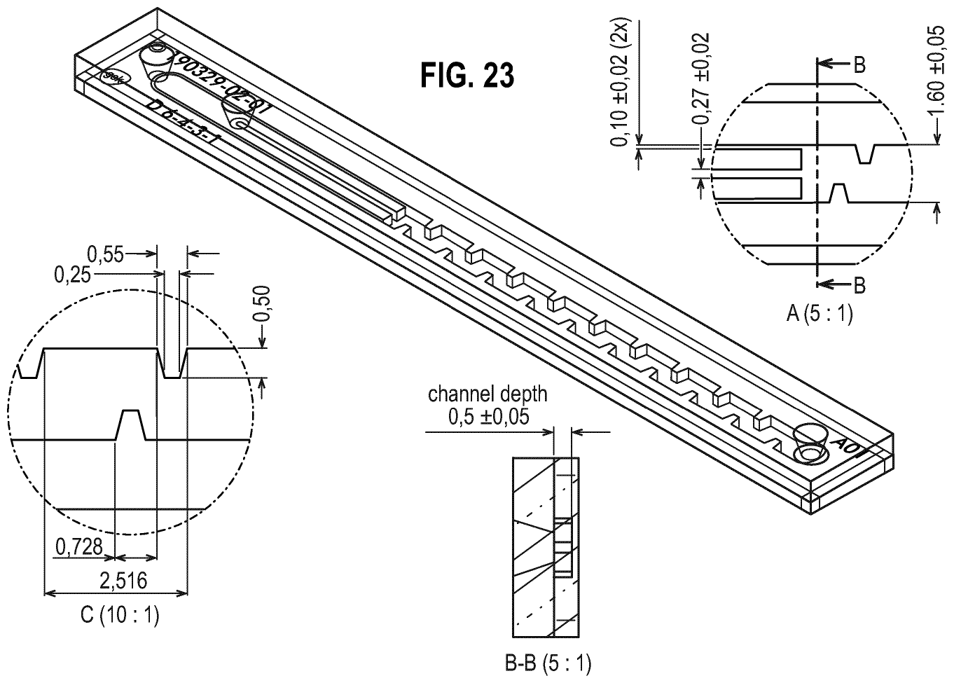

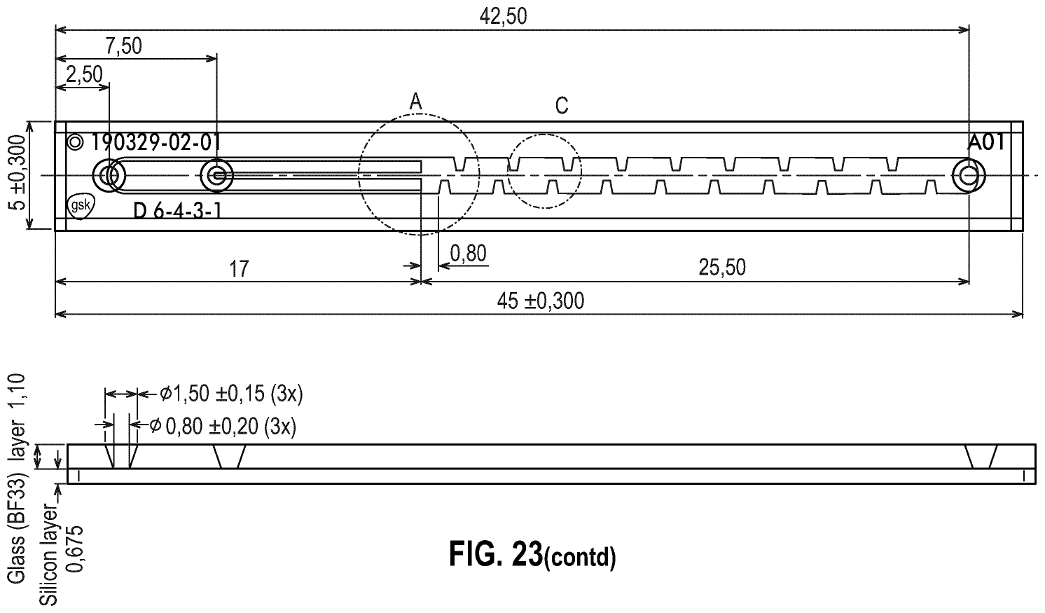

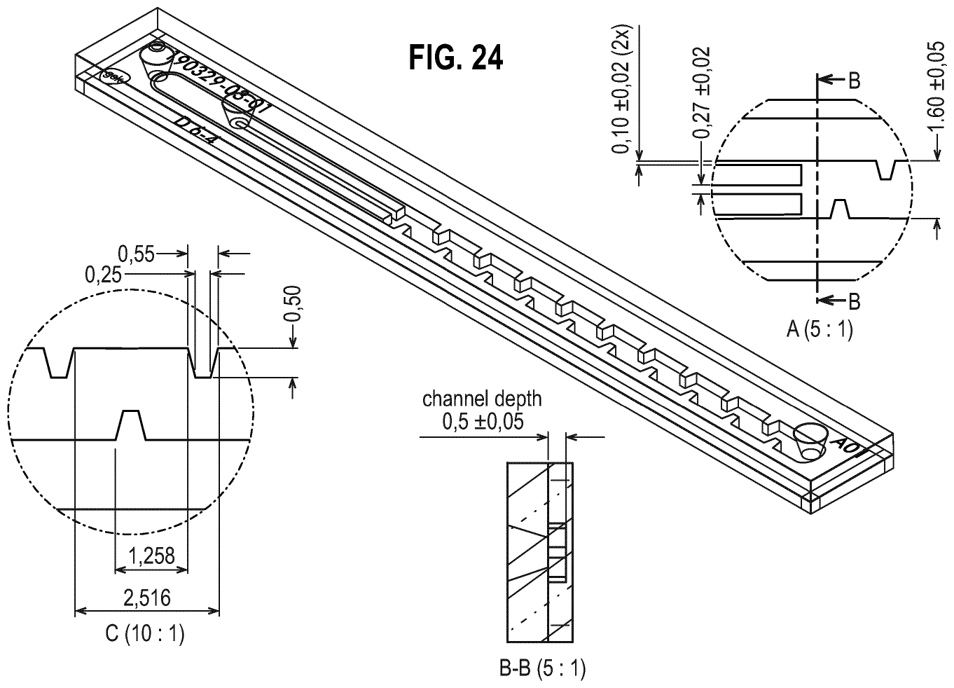

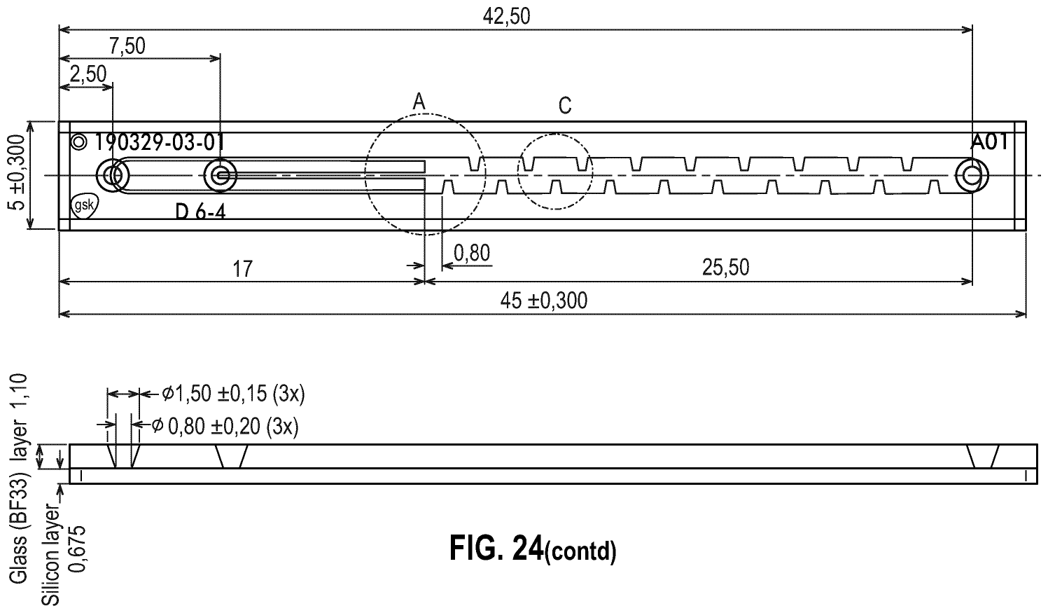

Schematics for Design 4-1 (from Example 7), Design 6-4-3-1 (Case 21 from Example 7), Design 6-4 (Case 9 from Example 7) and Design 6-5 (Case 10 from Example 7) are provided in FIG. 22 to FIG. 25. These designs were tested and compared to the design of WO2018219521 (Design 1′ as shown in FIG. 2) using the parameters:
total flow rate 16 ml/min
flow rate ratio 5 (i.e. 4:1 aqueous solution to organic solution)
temperature 20 degrees C.
organic phase ethanol/isopropanol 80:20 containing 3D-MPL 6.5 mg/ml, DOPC 130 mg/ml and cholesterol 32.5 mg/ml
aqueous phase containing QS-21 at 1.625 mg/ml

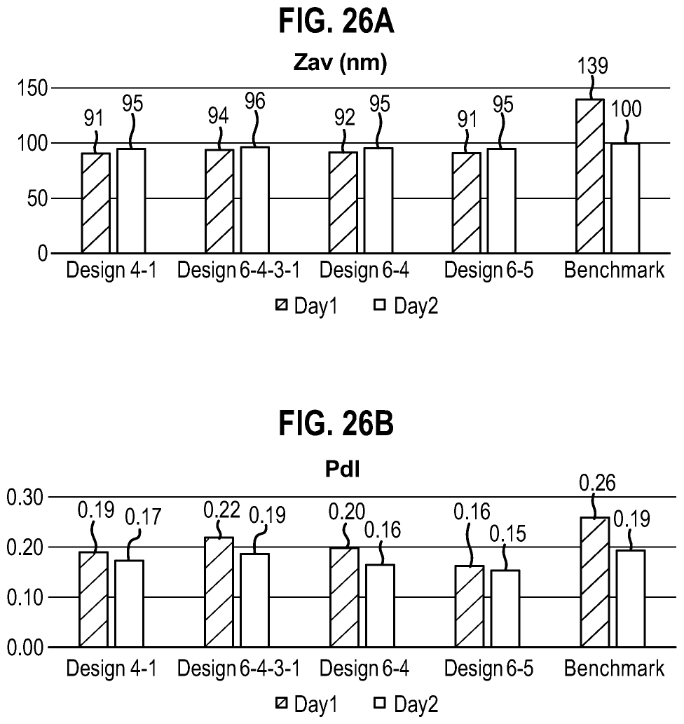

Product was tested using a Malvern Zeta Nano series for particle size and polydispersity, the results being shown in FIG. 26. The results show lower sizes for all new designs and a consistent lower polydispersity over 2 days of run for Designs 4-1 and 6-5.

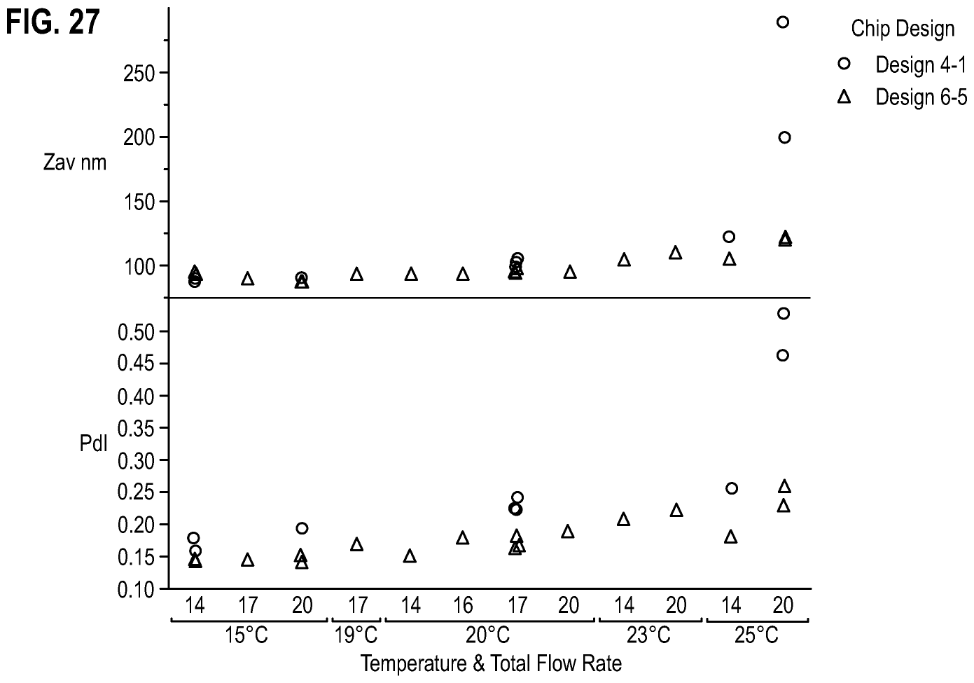

Designs 4-1 and 6-5 were then further investigated by varying temperature and total flow rate (flow rate ratio constant), the results being shown in FIG. 27. Statistical analysis of particle size found no significant difference between Designs 4-1 and 6-5 (p=0.16), although at 25 degrees and 20 ml/min Design 4-1 resulted in abnormally high values. Statistical analysis of PDI found a significant difference between Designs 4-1 and 6-5 (p=0.036), on average PDI was 0.05 higher with Design 4-1 than with Design 6-5.

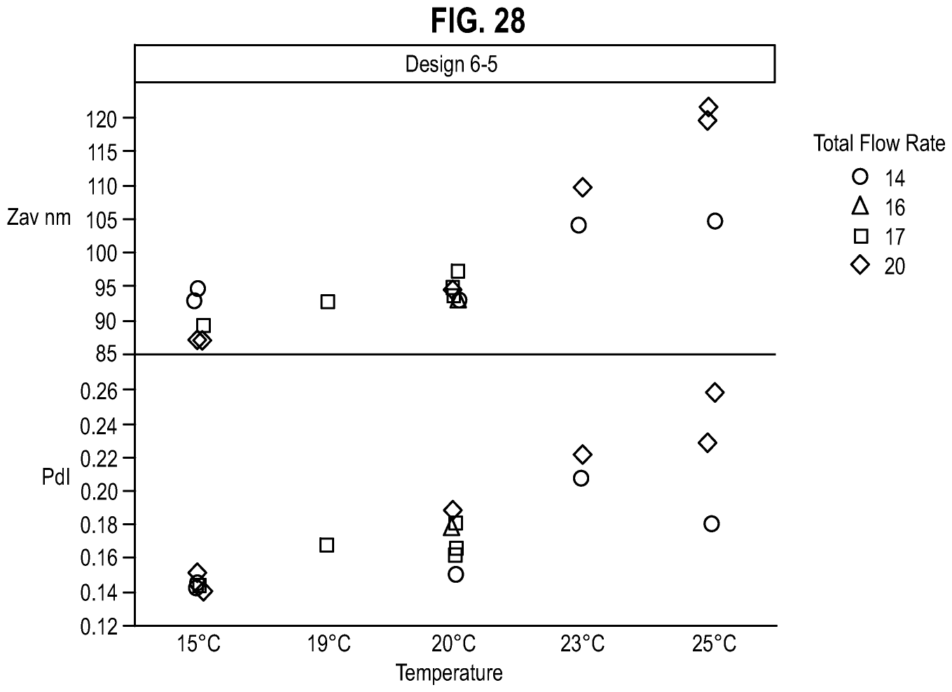

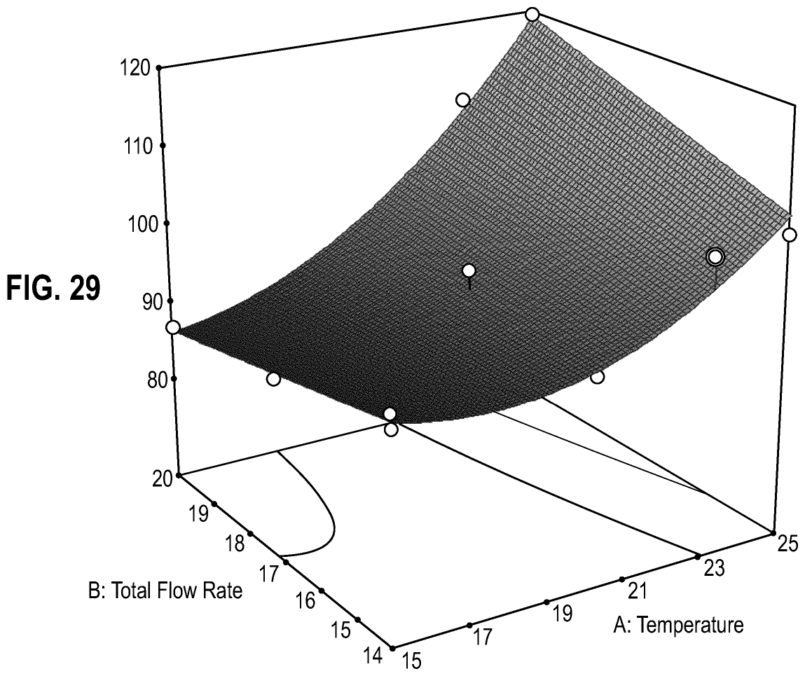

Additional testing was performed for Design 6-5 with varying temperature and flow rate. The results are shown in FIG. 28. A particle size design space was created based on these results, shown in FIG. 29.

Reynold's numbers were calculated for Design 4-1, Design 6-4, Design 6-4-3-1 and Design 6-5 based on the equation:

$$Re = \frac{{ρ UD}_{h}}{μ} = \frac{ρ U}{μ} \frac{2 wh}{w + h} = \frac{ρ}{μ} \frac{2 Q}{w + h}$$

where Q is the flow rate, u represents the fluid viscosity, w and h represent the channel width and height, p represents the fluid density, U represents the fluid average velocity. Density and viscosity were considered to be identical to water.

| | | Design | | |
|---|---|---|---|---|
| Location | Design 4-1 | 6-4-3-1 | Design 6-4 | Design 6-5 |
| Mixing chamber (general) | 355.6 | 254.0 | 254.0 | 254.0 |
| Mixing chamber (at baffle) | 463.8 | 333.3 | 333.3 | 333.3 |
| Second fluid inlets | 355.6 | 355.6 | 355.6 | 355.6 |
| First fluid inlet | 277.1 | 277.1 | 277.1 | 277.1 |

### Example 10—Scale-Up

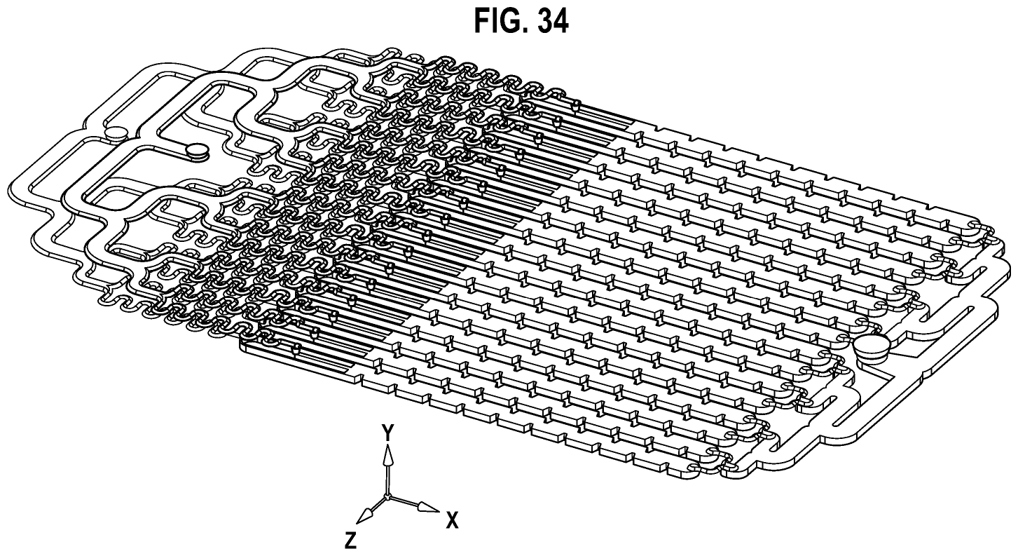

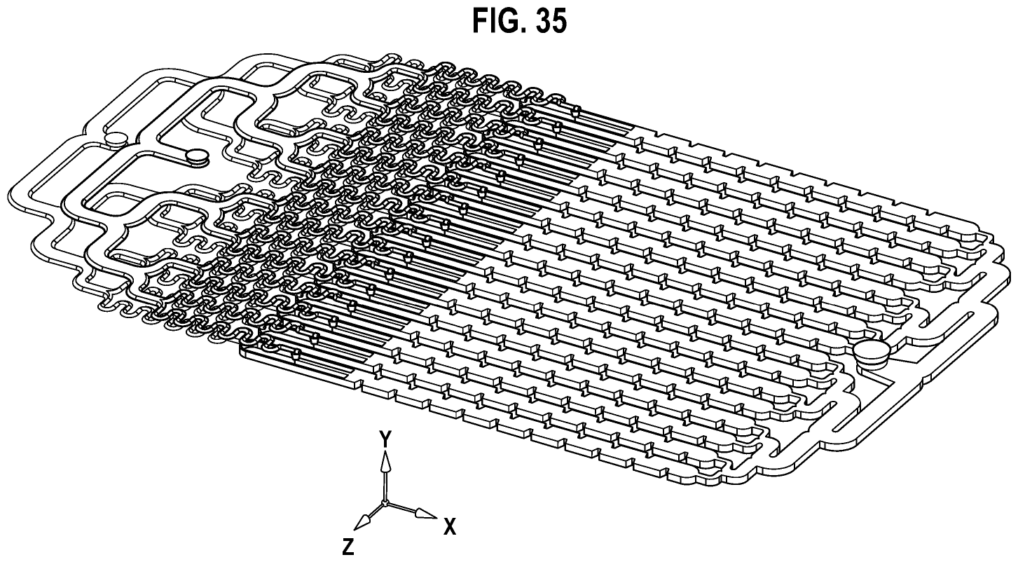

Two chips containing 16 mixing chambers according to Design 6-5 (Case 10 from Example 7) and incorporating integrated distribution and collection manifolds were prepared (see FIG. 34 and FIG. 35). Due to the requirement for overlapping channel paths, manifolds were incorporated in a plurality of chip layers. A first chip (Design 16A) had integrated inlet distribution in layers 2 and 3 and outlet collection in layer 1 with the outlet manifold connecting to the mixing chamber ends through channels located in layer 2. A second chip (Design 16C) had integrated inlet distribution in layers 2 and 3 and outlet collection in layer 1 with the outlet manifold connecting directly to the mixing chamber ends.

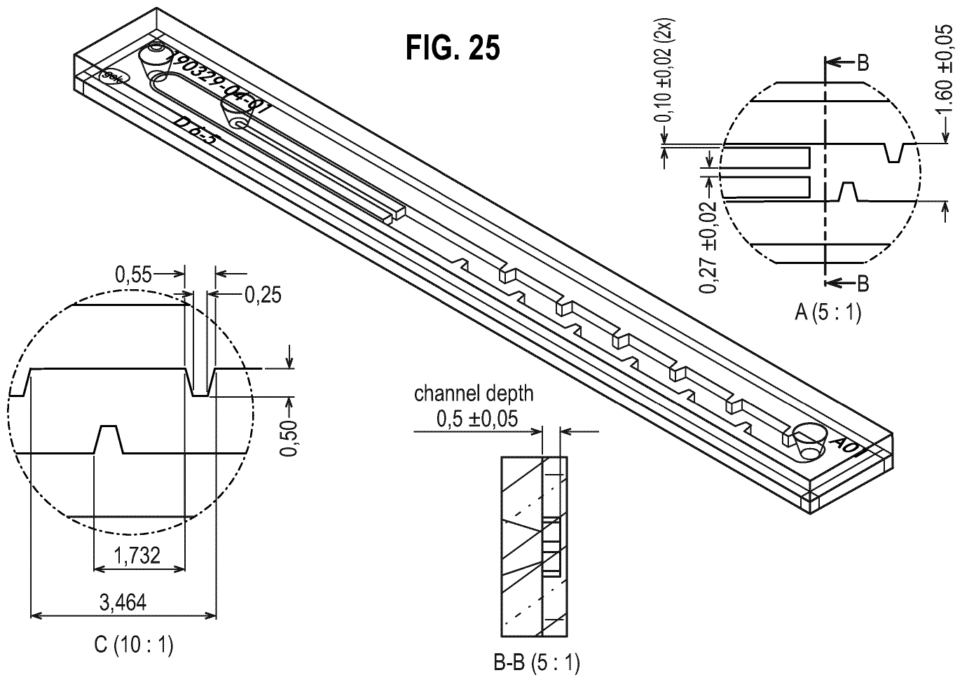

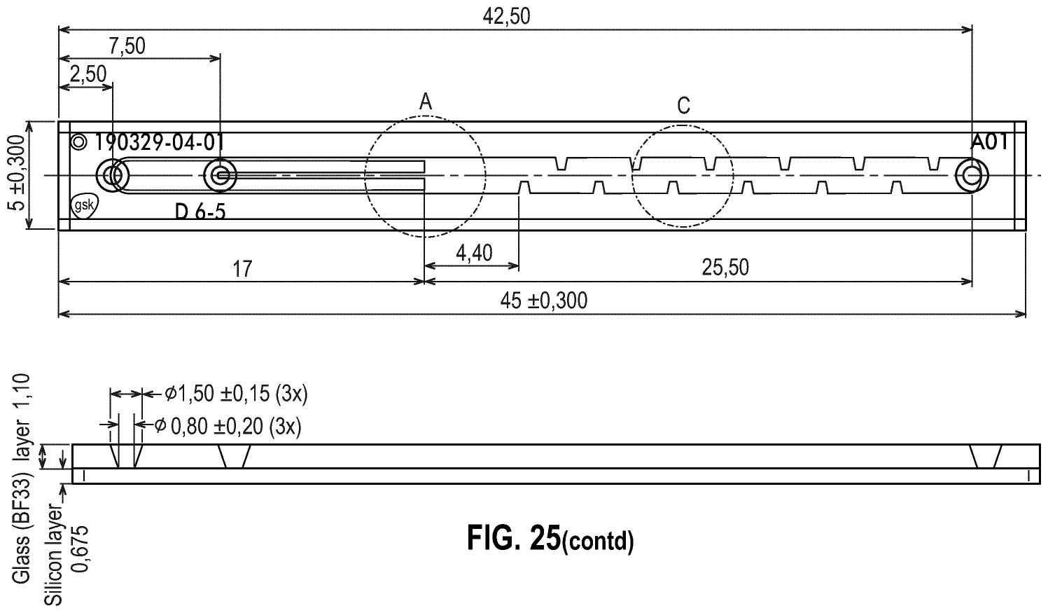

The multi-channel designs were tested and compared to the single channel Design 6-5 (FIG. 25) using the parameters:
total flow rate 16 ml/min per mixing chamber
flow rate ratio 5 (i.e. 4:1 aqueous solution to organic solution)
temperature 20 degrees C.
organic phase ethanol/isopropanol 80:20 containing 3D-MPL 6.5 mg/ml, DOPC 130 mg/ml and cholesterol 32.5 mg/ml.
aqueous phase containing QS-21 at 1.625 mg/ml

Product was tested using a Malvern Zeta Nano series for particle size and polydispersity.

| | 1-channel | | 16-channel A | | 16-channel C | |
|---|---|---|---|---|---|---|
| Run | Zav (nm) | Pdl | Zav (nm) | Pdl | Zav (nm) | Pdl |
| 1 | 99.5 | 0.18 | 99.3 | 0.16 | 98.8 | 0.16 |
| 2 | 100.6 | 0.19 | 98.5 | 0.15 | 99.3 | 0.17 |
| 3 | 99.3 | 0.19 | 99.6 | 0.16 | 97.9 | 0.17 |
| 4 | 98.9 | 0.18 | 99.8 | 0.17 | 99.8 | 0.18 |
| 5 | 101.8 | 0.19 | 100.1 | 0.16 | 98.5 | 0.16 |
| 6 | 101.7 | 0.18 | — | — | — | — |

Operation of both multi-channel configurations provided mixed material with low polydispersity.

### Example 11—Testing of Baffled Designs in the Manufacture of Liposomal Adjuvant

Design 6-5 (Case 10 from Example 7) was further investigated over a range of operating parameters:
total flow rate 12 to 16 ml/min
flow rate ratio 4 (i.e. 3:1 aqueous solution to organic solution)
flow rate sensitivity investigated by adjusting solution flow+/−1.2% from target
aqueous solution temperature 17 to 21 degrees C.
organic solution temperature 17 to 21 degrees C.
organic phase ethanol/isopropanol 80:20 containing 3D-MPL 6.5 mg/ml, DOPC 130 mg/ml and cholesterol 32.5 mg/ml
aqueous phase containing QS-21 at 2.167 mg/ml

Product was tested using a Malvern Zeta Nano series for particle size and polydispersity.

| | | | | | Variation | | |
|---|---|---|---|---|---|---|---|
| | T ° | | | | of | | |
| | Organic | T ° | Target Total | Variation of | Aqueous | Particle | |
| Run | Phase | Aqueous | Flow Rate | Organic phase | phase flow | size | |
| Id | (° C.) | Phase (° C.) | (mL/min) | flow rate (%) | rate (%) | (nm) | Pdi |
| 1 | 17 | 17 | 16 | −1, 2 | 1, 2 | 90 | 0.15 |
| 2 | 17 | 17 | 14 | 1, 2 | −1, 2 | 104 | 0.21 |
| 3 | 19 | 17 | 12 | −1, 2 | 1, 2 | 106 | 0.21 |
| 4 | 19 | 17 | 16 | 1, 2 | −1, 2 | 91 | 0.16 |
| 5 | 21 | 17 | 14 | −1, 2 | 1, 2 | 96 | 0.17 |
| 6 | 21 | 17 | 12 | 1, 2 | −1, 2 | 109 | 0.20 |
| 7 | 21 | 19 | 16 | 1, 2 | −1, 2 | 92 | 0.16 |
| 8 | 19 | 19 | 14 | 0 | 0 | 95 | 0.17 |
| 9 | 19 | 19 | 14 | 0 | 0 | 95 | 0.16 |
| 10 | 19 | 19 | 16 | −1, 2 | 1, 2 | 91 | 0.16 |
| 11 | 19 | 19 | 14 | 0 | 0 | 94 | 0.17 |
| 12 | 17 | 19 | 12 | 1, 2 | −1, 2 | 105 | 0.19 |
| 13 | 17 | 19 | 14 | −1, 2 | 1, 2 | 95 | 0.16 |
| 14 | 17 | 21 | 16 | 1, 2 | −1, 2 | 95 | 0.18 |
| 15 | 17 | 21 | 12 | −1, 2 | 1, 2 | 99 | 0.18 |
| 16 | 19 | 21 | 12 | 1, 2 | −1, 2 | 103 | 0.19 |
| 17 | 19 | 21 | 14 | −1, 2 | 1, 2 | 95 | 0.17 |
| 18 | 21 | 21 | 12 | −1, 2 | 1, 2 | 99 | 0.18 |
| 19 | 21 | 21 | 16 | −1, 2 | 1, 2 | 94 | 0.17 |
| 20 | 21 | 21 | 14 | 1, 2 | −1, 2 | 96 | 0.16 |

The application of which this description and claims forms part may be used as a basis for priority in respect of any subsequent application. The claims of such subsequent application may be directed to any feature or combination of features described herein. Embodiments are envisaged as being independently, fully combinable with one another where appropriate to the circumstances to form further embodiments of the invention. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### BIBLIOGRAPHY

Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144
Black, S., E. De Gregorio, and R. Rappuoli. 2015. Developing vaccines for an aging population. Science translational medicine. 7:281ps288
Coler R N et al. (2011) Development and Characterization of Synthetic Glucopyranosyl Lipid Adjuvant System as a Vaccine Adjuvant. PLoS ONE 6(1): e16333. doi:10.1371/journal.pone.0016333
Dalsgaard et al. in 1974 (“Saponin adjuvants”, Archiv. für die gesamte Virusforschung, Vol. 44, Springer Verlag, Berlin, p 243-254)
Dendouga, N., M. Fochesato, L. Lockman, S. Mossman, and S. L. Giannini. 2012. Cell-mediated immune responses to a varicella-zoster virus glycoprotein E vaccine using both a TLR agonist and QS-21 in mice. Vaccine. 30:3126-3135
Didierlaurent et al, 2017 Adjuvant system AS01: helping to overcome the challenges of modern vaccines Expert Reviews of Vaccines 16(1): 55-63
Galletti C et al 2012 Effect of inlet conditions on the engulfment pattern in a T-shaped micro-mixer Chemical Engineering Journal 300-313
Garcon, N., and M. Van Mechelen. 2011. Recent clinical experience with vaccines using MPL- and QS-21-containing adjuvant systems. Expert review of vaccines. 10:471-486
Han et al 2014 Characterization of the Structure and Immunostimulatory Activity of a Vaccine Adjuvant, De-O-Acylated Lipooligosaccharide PLoS ONE 9(1):e85838 (and associated correction Han et al 2014 PLoS ONE 9(4):94517)
Javid M. U et al, 2018 Analysis of Passive Mixing in a Serpentine Microchannel with Sinusoidal Side Walls Micromachines 9, 8
Kensil, C. R., U. Patel, M. Lennick, and D. Marciani. 1991. Separation and characterization of saponins with adjuvant activity from Quillaja saponaria Molina cortex. Journal of immunology. 146:431-437
Kensil, C. R., and R. Kammer. 1998. QS-21: a water-soluble triterpene glycoside adjuvant. Expert opinion on investigational drugs. 7:1475-1482
Kite G. C. et al. Metabolomic analysis of saponins in crude extracts of Quillaja saponaria by liquid chromatography/mass spectrometry for product authentication Rapid Communications in Mass Spectrometry 2004 18: 2859-2870
Nyberg N. et al. Separation and structural analysis of some saponins from Quillaja saponaria Molina Carbohydrate Research 2000 323:87-97
Nyberg N. et al. Solid-Phase Extraction NMR Studies of Chromatographic Fractions of Saponins from Quillaja Saponaria Anal. Chem. 2003 75 2:268-274
Ragupathi, G., J. R. Gardner, P. O. Livingston, and D. Y. Gin. 2011. Natural and synthetic saponin adjuvant QS-21 for vaccines against cancer. Expert review of vaccines. 10:463-470
Wong S. H. et al 2003 Investigation of mixing in a cross-shaped micromixer with static mixing elements for reaction kinetics studies Sensors and Actuators B: Chemical 95(1-3):414-424
WO94/21292
WO98/50399
WO2008153541
WO2009143457
WO2012080369
WO2015148764
WO2018219521
WO2019106192
WO2020109365
WO2020115178
EP1810746
GB 2 220 211 A
U.S. Pat. No. 4,436,727
U.S. Pat. No. 4,877,611
U.S. Pat. No. 4,866,034
U.S. Pat. No. 4,912,094
U.S. Pat. No. 6,303,347

1-23. (canceled)

24. A microfluidic mixing device comprising: a mixing chamber; one inlet channel into the mixing chamber for a first fluid and two inlet channels into the mixing chamber for a second fluid, said inlet channels being disposed substantially symmetrically at a proximal end of the mixing chamber; at least one outlet for mixed material at a distal end of the mixing chamber; characterised in that the mixing chamber comprises one or more baffles.

25. The device according to claim 24, wherein the inlet channel for the first fluid is 0.1 to 0.7 mm wide.

26. The device according to claim 24, wherein the inlet channels for the second fluid are 0.025 to 0.3 mm wide.

27. The device according to claim 24, wherein the direction of flow from the inlet channels for the first fluid and second fluid into the mixing chamber are substantially parallel to the general direction of flow in the mixing chamber.

28. The device according to claim 24, wherein the length of the mixing chamber is 15 to 100 mm.

29. The device according to claim 24, wherein the maximum width of the mixing chamber is 0.8 to 2.2 mm.

30. The device according to claim 24, wherein the minimum width of the mixing chamber is 0.8 to 2.2 mm.

31. The device according to claim 24, wherein the mixing chamber is 0.1 to 2 mm deep, such as 0.3 to 0.8 mm deep.

32. The device according to claim 24, wherein baffles are present on both sides of the mixing chamber.

33. The device according to claim 24, comprising 4 to 100 baffles.

34. The device according to claim 24, comprising baffles 0.1 to 1 mm wide.

35. The microfluidic mixing device according to claim 24 comprising:
a mixing chamber 25 mm in length, having a rectangular cross-section, parallel sides spaced 1.6 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;
characterised in that the mixing chamber comprises 12 baffles, the first baffle being located 4.4 mm from the proximal end of the mixing chamber, baffles being separated by 3.464 mm with an offset between the first and second sides of 1.732 mm, the baffles being trapezium in shape with a maximum length of 0.55 mm, a minimum length of 0.25 mm and a width of 0.5 mm.

36. The microfluidic mixing device according to claim 24 comprising:
a mixing chamber 25 mm in length, having a rectangular cross-section, parallel sides spaced 1 mm apart and parallel top and bottom walls spaced to provide a depth of 0.5 mm;
one inlet channel into the mixing chamber for a first fluid, being centrally located at a proximal end of the mixing chamber, having a rectangular cross-section, a width of 0.27 mm and a depth of 0.5 mm;
two inlet channels into the mixing chamber for a second fluid, being identical, located at each of the outer walls at the proximal end of the mixing chamber, having a rectangular cross-section, a width of 0.1 mm and a depth of 0.5 mm;
said inlet channels being disposed symmetrically at the proximal end of the mixing chamber and wherein the direction of flow from the inlet channels into the mixing chamber is parallel to the general direction of flow in the mixing chamber;
one outlet for mixed material at a distal end of the mixing chamber;
characterised in that the mixing chamber comprises 19 baffles, the first baffle being located 5.3 mm from the proximal end of the mixing chamber, baffles being separated by 2 mm with an offset between the first and second sides of 1 mm, the baffles being trapezium in shape with a maximum length of 0.33 mm, a minimum length of 0.15 mm and a width of 0.35 mm.

37. A method of manufacturing a liposomal adjuvant using a microfluidic device according to claim 24, comprising the following steps:
(a) mixing in the device a first solution comprising a solvent and lipid, and a second solution comprising water; and
(b) removing the solvent.

38. A method of manufacturing a liposomal concentrate of use in the preparation of a liposomal adjuvant using a microfluidic device according to claim 24, comprising the step of mixing in the device a first solution comprising a solvent and lipid, and a second solution comprising water.

39. The method of claim 37, further comprising the step of mixing the liposomal adjuvant with an immunogen or antigen, or a polynucleotide encoding the immunogen or antigen.

40. A method for the manufacture of an adjuvanted immunogenic composition, said method comprising the step of combining an immunogen or antigen, or a polynucleotide encoding the immunogen or antigen, with a liposomal adjuvant manufactured according to the method of claim 37.

41. A liposomal adjuvant comprising a saponin, TLR4 agonist, DOPC and sterol produced according to the method of claim 37.

42. An adjuvanted immunogenic composition produced according to the method of claim 39.

43. A liposome containing solution obtainable by, such as obtained by, mixing the first solution and second solution according to the method of claim 37 prior to the removal of solvent.
